(19) United States
(12) Patent Application Publication
(10) Pub. No.: US 2025/0017935 A1
(43) Pub. Date: Jan. 16, 2025

(54) 7,8-DIHYDRO-5H-1,6-NAPHTHYRIDINE DERIVATIVES AS POSITIVE ALLOSTERIC MODULATORS OF THE MUSCARINIC ACETYLCHOLINE RECEPTOR M4 FOR TREATING NEUROLOGICAL AND PSYCHIATRIC DISORDERS

(71) Applicant: Vanderbilt University, Nashville, TN (US)

(72) Inventors: Craig W. Lindsley, Brentwood, TN (US); P. Jeffrey Conn, Nashville, TN (US); Darren W. Engers, Brentwood, TN (US); Julie L. Engers, Brentwood, TN (US); Madeline F. Long, Nashville, TN (US); Charlotte Park, Nashville, TN (US); Aaron M. Bender, Spring Hill, TN (US); Logan A. Baker, Thompson's Station, TN (US)

(21) Appl. No.: 18/700,120

(22) PCT Filed: Oct. 14, 2022
(86) PCT No.: PCT/US2022/046762

§ 371 Date: Apr. 10, 2024

Related U.S. Application Data
(60) Provisional application No. 63/255,902, filed on Oct. 14, 2021.

(51) Int. Cl. A61K 31/519; A61K 31/517; A61P 25/18; A61P 25/28; C07D 471/04; C07D 519/00

(52) U.S. Cl. CPC A61K 31/519; A61K 31/517; A61P 25/18; A61P 25/28; C07D 471/04; C07D 519/00

(57) ABSTRACT

The present invention relates to 7,8-dihydro-5H-1,6-naphthyridine derivatives of formula (I) The present compounds are positive allosteric modulators of the muscarinic acetylcholine receptor M4 (mAChR M4) for use in treating neurological and psychiatric disorders associated with muscarinic acetylcholine receptor dysfunction. An exemplary compound is e.g. example 1: 5,6-dimethyl-4-[3-(trifluoromethyl)-7,8-dihydro-5H-1,6-naphthyridin-6-yl]thieno[2,3-d]pyrimidine (compound 1): Data on the activity of exemplary compounds in an mAChR M4 cell-based assay is provided.

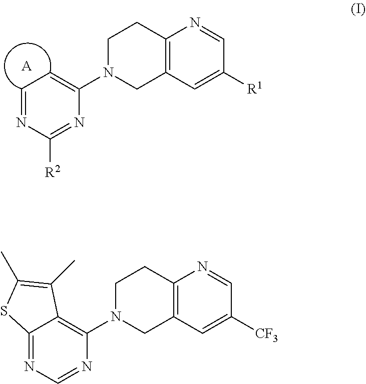

### RELATED APPLICATIONS

This application claims priority to U.S. Provisional Application No. 63/255,902, filed Oct. 14, 2021, which is hereby incorporated by reference in its entirety.

### STATEMENT OF GOVERNMENT INTEREST

This invention was made with government support under grant numbers 5R01MH073676 and 1U19MH106839-01 awarded by the National Institutes of Health. The government has certain rights in the invention.

### TECHNICAL FIELD

The present invention relates to compounds, compositions, and methods for treating neurological and psychiatric disorders associated with muscarinic acetylcholine receptor dysfunction.

### BACKGROUND

Cholinergic neurotransmission involves the activation of nicotinic acetylcholine receptors (nAChRs) or the muscarinic acetylcholine receptors (mAChRs) by the binding of the endogenous orthosteric agonist acetylcholine (ACh). Conditions associated with cognitive impairment, such as Alzheimer's disease, are accompanied by a reduction of acetylcholine content in the brain. This is believed to be the result of degeneration of cholinergic neurons of the basal forebrain, which widely innervate multiple areas of the brain, including the association cortices and hippocampus, which are critically involved in higher processes. Clinical data supports that cholinergic hypofunction contributes to the cognitive deficits of patients suffering from schizophrenia. Efforts to increase acetylcholine levels have focused on increasing levels of choline, the precursor for acetylcholine synthesis, and on blocking acetylcholinesterase (AChE), the enzyme that metabolizes acetylcholine. As a result, acetylcholinesterase (AChE) inhibitors, which inhibit the hydrolysis of ACh, have been approved in the United States for use in the palliative, but not disease-modifying, treatment of the cognitive deficits in AD patients.

Attempts to augment central cholinergic function through the administration of choline or phosphatidylcholine have not been successful. AChE inhibitors have shown therapeutic efficacy, but have been found to have frequent cholinergic side effects due to peripheral acetylcholine stimulation, including abdominal cramps, nausea, vomiting, and diarrhea. These gastrointestinal side effects have been observed in about a third of the patients treated. In addition, some AChE inhibitors, such as tacrine, have also been found to cause significant hepatotoxicity with elevated liver transaminases observed in about 30% of patients. The adverse effects of AChE inhibitors have severely limited their clinical utility. An alternative approach to pharmacologically target cholinergic hypofunction is the activation of mAChRs, which are widely expressed throughout the body.

The mAChRs are members of the class A G protein-coupled receptors (GPCRs) and include five subtypes, designated M₁-M₅. The M₁, M₃ and M₅ subtypes mainly couple to G_{q} and activate phospholipase C, whereas the M₂ and M₄ subtypes mainly couple to G_{i/o} and associated effector systems. These five distinct mAChR subtypes have been identified in the mammalian central nervous system where they are prevalent and differentially expressed. M₁-M₅ have varying roles in cognitive, sensory, motor and autonomic functions. Thus, without wishing to be bound by a particular theory, it is believed that selective agonists of mAChR subtypes that regulate processes involved in cognitive function could prove to be superior therapeutics for treatment of psychosis, schizophrenia and related disorders. The muscarinic M₄ receptor has been shown to have a major role in cognitive processing and is believed to have a major role in the pathophysiology of psychotic disorders, including schizophrenia.

Evidence suggests that the most prominent adverse effects of AChE inhibitors and other cholinergic agents are mediated by activation of peripheral M₂ and M₃ mAChRs and include bradycardia, GI distress, excessive salivation, and sweating. In contrast, M₄ has been viewed as the most likely subtype for mediating the effects of muscarinic acetylcholine receptor dysfunction in psychotic disorders, including schizophrenia, cognition disorders, and neuropathic pain. Because of this, considerable effort has been focused on developing selective M₄ agonists for treatment of these disorders. Unfortunately, these efforts have been largely unsuccessful because of an inability to develop compounds that are highly selective for the mAChR M₄. Because of this, mAChR agonists that have been tested in clinical studies induce a range of adverse effects by activation of peripheral mAChRs. To fully understand the physiological roles of individual mAChR subtypes and to further explore the therapeutic utility of mAChR ligands in psychosis, including schizophrenia, cognition disorders and other disorders, it can be important to develop compounds that are highly selective activators of mAChR M₄ and other individual mAChR subtypes.

Previous attempts to discover and develop agonists that are highly selective for individual mAChR subtypes have failed because of the high conservation of the orthosteric ACh binding site. It is believed that developing compounds that act at allosteric sites on mAChRs that are removed from the orthosteric site and are less highly conserved could circumvent problems associated with targeting the highly conserved orthosteric ACh binding site. This approach has improved the development of selective ligands for multiple GPCR subtypes. In the case of mAChRs, a major goal has been to develop allosteric ligands that selectively increase activity of mAChR M₄ or other mAChR subtypes. Allosteric activators can include allosteric agonists, that act at a site removed from the orthosteric site to directly activate the receptor in the absence of ACh as well as positive allosteric modulators (PAMs), which do not activate the receptor directly but potentiate activation of the receptor by the endogenous orthosteric agonist ACh. Also, it is possible for a single molecule to have both allosteric potentiator and allosteric agonist activity.

More recently, muscarinic agonists including xanomeline have been shown to be active in animal models with similar profiles to known antipsychotic drugs, but without causing 20 catalepsy (Bymaster et al., Eur. J. Pharmacol. 1998, 356, 109, Bymaster et al., Life Sci. 1999, 64, 527; Shannon et al., J. Pharmacol. Exp. Ther. 1999, 290, 901; Shannon et al., Schizophrenia Res. 2000, 42, 249). Further, xanomeline was shown to reduce psychotic behavioral symptoms such as delusions, suspiciousness, vocal outbursts, and hallucinations in Alzheimer's disease patients (Bodick et al., Arch. Neurol. 1997, 54, 465), however treatment induced side effects, e.g., gastrointestinal effects, have severely limited the clinical utility of this compound.

Despite advances in muscarinic acetylcholine receptor research, there is still a scarcity of compounds that are potent, efficacious, and selective activators of the M₄ mAChR and also effective in the treatment of neurological and psychiatric disorders associated with cholinergic activity and diseases in which the muscarinic M₄ receptor is involved.

### SUMMARY

In one aspect, the present invention provides is a compound of formula (I), or a pharmaceutically acceptable salt thereof, wherein:

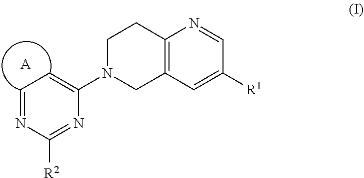

A is selected from a 5-membered heteroarene, a 5-membered heterocycle, and a 5-membered alicyclic ring system, wherein the heteroarene and heterocycle contain 1 to 2 heteroatoms independently selected from the group consisting of N, O, and S;
wherein the 5-membered heteroarene, the 5-membered heterocycle, and the 5-membered alicyclic ring system of A are unsubstituted or substituted with 1, 2 or 3 substituents independently selected from C₁₋₃ alkyl, C₁₋₄ fluoroalkyl, C₃₋₆ cycloalkyl, and halogen;
R¹ is selected from C₁₋₄ fluoroalkyl, C₃₋₆ cycloalkyl, phenyl, 5- or 6-membered heteroaryl, —NR^{a}R^{b} and -LR^{c};
L is CH₂ or O;
wherein R^{a} is a 5- or 6-membered heteroaryl or phenyl;
wherein R^{b} is hydrogen; or
wherein R^{a} and R^{b} together with the N to which they are attached form a heterocyclyl;
wherein R^{c} is 5- or 6-membered heteroaryl or phenyl;
wherein each phenyl, 5- or 6-membered heteroaryl, cycloalkyl, and heterocyclyl of R¹, R^{a}, or R^{c} is unsubstituted or substituted with 1, 2, or 3 substituents independently selected from C₁₋₄ alkyl, C₁₋₄ fluoroalkyl, —NR^{d}R^{e}, halogen, and C₃₋₆ cycloalkyl;
wherein R^{d} and R^{e} are independently selected from hydrogen and C₁₋₄ alkyl;
R² is selected from hydrogen, C₁₋₄ alkyl, halogen, —NR^{g}R^{h}, —ORⁱ and C₃₋₆ cycloalkyl;
wherein R^{g} is independently selected from C₁₋₄ alkyl, C₁₋₃ hydroxyalkyl, C₃₋₆ cycloalkyl, phenyl, and 5- or 6-membered heteroaryl;
wherein R^{h} is independently selected from hydrogen and C₁₋₄ alkyl; or
wherein R^{g} and R^{h} together with the N to which they are attached form a heterocyclyl;
wherein Rⁱ is C₁₋₄ alkyl;
wherein each 5- or 6-membered heteroaryl, heterocyclyl, phenyl, and C₃₋₆ cycloalkyl of R² is unsubstituted or substituted with 1, 2, or 3 substituents independently selected from —NR^{j}R^{k}, C₁₋₄ fluoroalkyl, hydroxyl, C₁₋₄ alkyl, halogen, C₃₋₆ cycloalkyl; and
wherein Rⁱ and R^{k} are independently selected from hydrogen and C₁₋₄ alkyl.

In another aspect, the invention provides a pharmaceutical composition comprising a compound of the above formula (I) and pharmaceutically acceptable salts thereof together with a pharmaceutically acceptable carrier.

In a further aspect, the invention provides compounds of the above formula (I) and pharmaceutically acceptable salts thereof for use in therapy.

In a further aspect, the invention provides compounds of the above formula (I) and pharmaceutically acceptable salts thereof for use in a method for the treatment of a disease associated with mAChR M₄ dysfunction.

In a further aspect, the invention relates to the use of a compound of the above formula (I) and pharmaceutically acceptable salts thereof in the manufacture of a medicament for use in the treatment of a disorder associated with mAChR M₄ dysfunction.

In a further aspect, the invention relates to a method for the treatment of a disorder associated with mAChR M₄ dysfunction, the method comprising the administration of a therapeutically effective amount of a compound of the above formula (I), or pharmaceutically acceptable salts thereof, to a patient in need thereof.

In a further aspect, the invention relates to a kit comprising the compounds of the invention.

### BRIEF DESCRIPTION OF THE DRAWINGS

FIG. 1 shows a schematic illustration of ligand binding sites, including the orthosteric site and an allosteric site, in the muscarinic acetylcholine receptor M4.

### DETAILED DESCRIPTION

The invention will now be described in greater details. Each specific embodiment and variation of features applies equally to each aspect of the invention unless specifically stated otherwise. Disclosed herein are positive allosteric modulators (i.e., potentiators) of the muscarinic acetylcholine receptor M₄ (mAChR M₄), methods of making same, pharmaceutical compositions comprising same, and methods of treating neurological and psychiatric disorders associated with muscarinic acetylcholine receptor dysfunction using same. The compounds include analogs of 6-(pyrimidin-4-yl)-5,6,7,8-tetrahydroquinoline compounds, as an example 5,6-dimethyl-4-[3-(trifluoromethyl)-7,8-dihydro-5H-1,6-naphthyridin-6-yl]thieno[2,3-d]pyrimidine.

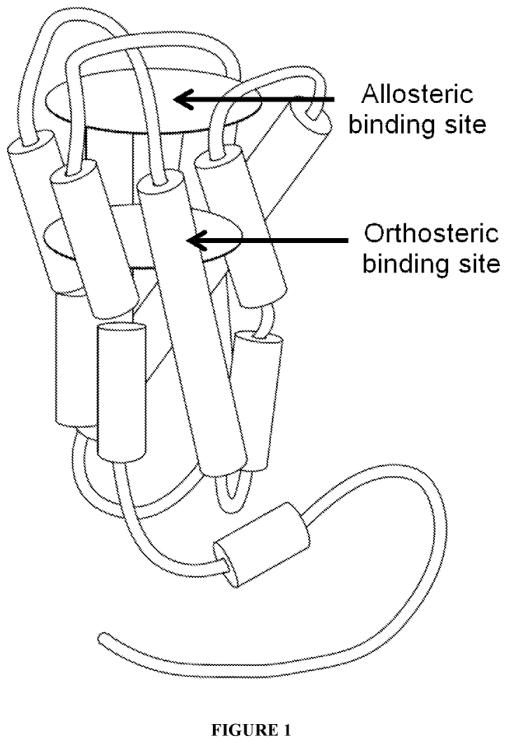

The human muscarinic acetylcholine receptor M₄ (mAChR M₄) is a protein of 479 amino acids encoded by the CHRM4 gene. The molecular weight of the unglycosylated protein is about 54 kDa and it is a transmembrane GPCR. As described above, the mAChR M₄ is a member of the GPCR Class A, or the rhodopsin-like GPCRs, which are characterized by structural features similar to rhodopsin such as seven transmembrane segments. The muscarinic acetylcholine receptors have the N-terminus oriented to the extracellular face of the membrane and the C-terminus located on the cytoplasmic face. A schematic of the structure of mAChR M₄ is shown in FIG. 1, with the transmembrane segments shown as cylindrical shapes (which span the lipid bilayer of the cell membrane). The orthosteric binding for natural ligand, acetylcholine, for mAChRs is within a pocket located in the transmembrane segments as depicted in FIG. 1.

Previous attempts to discover and develop agonists that are highly selective for individual mAChR subtypes have largely failed because of the high conservation of the orthosteric ACh binding site. It is believed that developing compounds that act at allosteric sites on mAChRs that are removed from the orthosteric site and are less highly conserved could circumvent problems associated with targeting the highly conserved orthosteric ACh binding site. Without wishing to be bound by a particular theory, the compound of the inventions and products of the disclosed methods are believed to bind to an allosteric site distinct from the orthosteric binding site. For example, a compound of the invention can bind at the binding site as illustrated in FIG. 1.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present invention also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this invention, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5th Edition, John Wiley & Sons, Inc., New York, 2001; Larock, Comprehensive Organic Transformations, VCH Publishers, Inc., New York, 1989; Carruthers, Some Modern Methods of Organic Synthesis, 3rd Edition, Cambridge University Press, Cambridge, 1987; the entire contents of each of which are incorporated herein by reference.

The term “acetoxyalkyl,” as used herein, means at least one acetoxy group (—OC(O)CH₃), is appended to the parent molecular moiety through an alkylene group, as defined herein.

The term “alkoxy,” as used herein, refers to a group alkyl-O—. Representative examples of alkoxy include, but are not limited to, methoxy, ethoxy, propoxy, 2-propoxy, butoxy and tert-butoxy.

The term “alkyl,” as used herein, means a straight or branched, saturated hydrocarbon chain. The term “lower alkyl” or “C₁₋₆alkyl” means a straight or branched chain hydrocarbon containing from 1 to 6 carbon atoms. The term “C₁₋₄alkyl” means a straight or branched chain saturated hydrocarbon containing from 1 to 4 carbon atoms. Representative examples of alkyl include, but are not limited to, methyl, ethyl, n-propyl, iso-propyl, n-butyl, sec-butyl, isobutyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl,” as used herein, means a straight or branched, hydrocarbon chain containing at least one carbon-carbon double bond and from 2 to 10 carbon atoms.

The term “alkoxyalkyl,” as used herein, refers to an alkoxy group, as defined herein, appended to the parent molecular moiety through an alkyl group, as defined herein.

The term “alkoxyfluoroalkyl,” as used herein, refers to an alkoxy group, as defined herein, appended to the parent molecular moiety through a fluoroalkyl group, as defined herein.

The term “phenoxyl”, as used herein, refers to an optional substituted phenyl group appended to the parent molecular moiety through an oxygen atom. Representative examples of phenoxy include, but are not limited to 2-fluorophenoxy, 2-fluoro-4-methyl-phenoxy, 2-fluoro-5-methyl-phenoxy.

The term “alkylene,” as used herein, refers to a divalent group derived from a straight or branched saturated chain hydrocarbon, for example, of 1 to 6 carbon atoms. Representative examples of alkylene include, but are not limited to, —CH₂—, —CH₂CH₂—, —CH₂CH₂CH₂—, —CH₂CH(CH₃)CH₂—, —CH₂CH₂CH₂CH₂—, —CH₂CH(CH₃)CH₂CH₂—, and —CH₂CH₂CH₂CH₂CH₂—.

The term “alkylamino,” as used herein, means at least one alkyl group, as defined herein, is appended to the parent molecular moiety through an amino group, as defined herein.

The term “amide,” as used herein, means —C(O)NR— or —NRC(O)—, wherein R may be hydrogen, alkyl, cycloalkyl, aryl, heteroaryl, heterocycle, alkenyl, or heteroalkyl.

The term “aminoalkyl,” as used herein, means at least one amino group, as defined herein, is appended to the parent molecular moiety through an alkylene group, as defined herein.

The term “amino,” as used herein, means —NR^{x}R^{y}, wherein each Rx and Ry may independently be hydrogen, alkyl, cycloalkyl, aryl, heteroaryl, heterocycle, alkenyl, or heteroalkyl. In some embodiments, Rx and Ry together with the N to which they are attached to form a heterocycle as defined herein. In the case of an aminoalkyl group or any other moiety where amino appends together two other moieties, amino may be —NR^{x}—, wherein R^{x} may be hydrogen, alkyl, cycloalkyl, aryl, heteroaryl, heterocycle, alkenyl, or heteroalkyl.

The term “aryl,” as used herein, refers to a phenyl or a phenyl appended to the parent molecular moiety and fused to a cycloalkane group (e.g., the aryl may be indan-4-yl), fused to a 6-membered arene group (i.e., the aryl is naphthyl), or fused to a non-aromatic heterocycle (e.g., the aryl may be benzo[d][1,3]dioxol-5-yl). The term “phenyl” is used when referring to a substituent and the term 6-membered arene is used when referring to a fused ring. The 6-membered arene is monocyclic (e.g., benzene or benzo). The aryl may be monocyclic (phenyl) or bicyclic (e.g., a 9- to 12-membered fused bicyclic system).

The term “cyanoalkyl,” as used herein, means at least one —CN group, is appended to the parent molecular moiety through an alkylene group, as defined herein.

The term “cyanofluoroalkyl,” as used herein, means at least one —CN group, is appended to the parent molecular moiety through a fluoroalkyl group, as defined herein.

The term “cycloalkoxy,” as used herein, refers to a cycloalkyl group, as defined herein, appended to the parent molecular moiety through an oxygen atom.

The term “alicyclic ring system” refers to a carbocyclic ring structure containing three to ten carbon atoms, which may be saturated or unsaturated, but may not be a benzenoid or other aromatic ring system.

The term “cycloalkyl” or “cycloalkane,” as used herein, refers to a saturated ring system containing all carbon atoms as ring members and zero double bonds. The term “cycloalkyl” is used herein to refer to a cycloalkane when present as a substituent. A cycloalkyl may be a monocyclic cycloalkyl (e.g., cyclopropyl), a fused bicyclic cycloalkyl (e.g., decahydronaphthalenyl), or a bridged cycloalkyl in which two non-adjacent atoms of a ring are linked by an alkylene bridge of 1, 2, 3, or 4 carbon atoms (e.g., bicyclo[2.2.1]heptanyl). Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, and bicyclo[1.1.1]pentanyl.

The term “cycloalkenyl” or “cycloalkene,” as used herein, means a non-aromatic monocyclic or multicyclic ring system containing all carbon atoms as ring members and at least one carbon-carbon double bond and preferably having from 5-10 carbon atoms per ring. The term “cycloalkenyl” is used herein to refer to a cycloalkene when present as a substituent. A cycloalkenyl may be a monocyclic cycloalkenyl (e.g., cyclopentenyl), a fused bicyclic cycloalkenyl (e.g., octahydronaphthalenyl), or a bridged cycloalkenyl in which two non-adjacent atoms of a ring are linked by an alkylene bridge of 1, 2, 3, or 4 carbon atoms (e.g., bicyclo[2.2.1]heptenyl). Exemplary monocyclic cycloalkenyl rings include cyclopentenyl, cyclohexenyl or cycloheptenyl. Exemplary monocyclic cycloalkenyl rings include cyclopentenyl, cyclohexenyl or cycloheptenyl.

The term “fluoroalkyl,” as used herein, means an alkyl group, as defined herein, in which one, two, three, four, five, six, seven or eight hydrogen atoms are replaced by fluorine. Representative examples of fluoroalkyl include, but are not limited to, 2-fluoroethyl, 2,2,2-trifluoroethyl, trifluoromethyl, difluoromethyl, pentafluoroethyl, and trifluoropropyl such as 3,3,3-trifluoropropyl.

The term “fluoroalkoxy,” as used herein, means at least one fluoroalkyl group, as defined herein, is appended to the parent molecular moiety through an oxygen atom. Representative examples of fluoroalkoxy include, but are not limited to, difluoromethoxy, trifluoromethoxy and 2,2,2-trifluoroethoxy.

The term “halogen” or “halo,” as used herein, means Cl, Br, I, or F.

The term “haloalkyl,” as used herein, means an alkyl group, as defined herein, in which one, two, three, four, five, six, seven or eight hydrogen atoms are replaced by a halogen.

The term “haloalkoxy,” as used herein, means at least one haloalkyl group, as defined herein, is appended to the parent molecular moiety through an oxygen atom.

The term “halocycloalkyl,” as used herein, means a cycloalkyl group, as defined herein, in which one or more hydrogen atoms are replaced by a halogen.

The term “heteroalkyl,” as used herein, means an alkyl group, as defined herein, in which one or more of the carbon atoms has been replaced by a heteroatom independently selected from S, O, P and N. Representative examples of heteroalkyls include, but are not limited to, alkyl ethers, secondary and tertiary alkyl amines, amides, and alkyl sulfides.

The term “heteroaryl,” as used herein, refers to an aromatic monocyclic heteroatom-containing ring (monocyclic heteroaryl) or a bicyclic ring system containing at least one monocyclic heteroaromatic ring (bicyclic heteroaryl). The term “heteroaryl” is used herein to refer to a heteroarene when present as a substituent. The monocyclic heteroaryl are five or six membered rings containing at least one heteroatom independently selected from the group consisting of N, O and S (e.g. 1, 2, 3, or 4 heteroatoms independently selected from O, S, and N). The five membered aromatic monocyclic rings have two double bonds and the six membered aromatic monocyclic rings have three double bonds. The bicyclic heteroaryl is an 8- to 12-membered ring system and includes a fused bicyclic heteroaromatic ring system (i.e., 10π electron system) such as a monocyclic heteroaryl ring fused to a 6-membered arene (e.g., quinolin-4-yl, indol-1-yl), a monocyclic heteroaryl ring fused to a monocyclic heteroarene (e.g., naphthyridinyl), and a phenyl fused to a monocyclic heteroarene (e.g., quinolin-5-yl, indol-4-yl). A bicyclic heteroaryl/heteroarene group includes a 9-membered fused bicyclic heteroaromatic ring system having four double bonds and at least one heteroatom contributing a lone electron pair to a fully aromatic 10π electron system, such as ring systems with a nitrogen atom at the ring junction (e.g., imidazopyridine) or a benzoxadiazolyl. A bicyclic heteroaryl also includes a fused bicyclic ring system composed of one heteroaromatic ring and one non-aromatic ring such as a monocyclic heteroaryl ring fused to a monocyclic carbocyclic ring (e.g., 6,7-dihydro-5H-cyclopenta[b]pyridinyl), or a monocyclic heteroaryl ring fused to a monocyclic heterocycle (e.g., 2,3-dihydrofuro[3,2-b]pyridinyl). The bicyclic heteroaryl is attached to the parent molecular moiety at an aromatic ring atom. Other representative examples of heteroaryl include, but are not limited to, indolyl (e.g., indol-1-yl, indol-2-yl, indol-4-yl), pyridinyl (including pyridin-2-yl, pyridin-3-yl, pyridin-4-yl), pyrimidinyl, pyrazinyl, pyridazinyl, pyrazolyl (e.g., pyrazol-4-yl), pyrrolyl, benzopyrazolyl, 1,2,3-triazolyl (e.g., triazol-4-yl), 1,3,4-thiadiazolyl, 1,2,4-thiadiazolyl, 1,3,4-oxadiazolyl, 1,2,4-oxadiazolyl, imidazolyl, thiazolyl (e.g., thiazol-4-yl), isothiazolyl, thienyl, benzimidazolyl (e.g., benzimidazol-5-yl), benzothiazolyl, benzoxazolyl, benzoxadiazolyl, benzothienyl, benzofuranyl, isobenzofuranyl, furanyl, oxazolyl, isoxazolyl, purinyl, isoindolyl, quinoxalinyl, indazolyl (e.g., indazol-4-yl, indazol-5-yl), quinazolinyl, 1,2,4-triazinyl, 1,3,5-triazinyl, isoquinolinyl, quinolinyl, imidazo[1,2-a]pyridinyl (e.g., imidazo[1,2-a]pyridin-6-yl), naphthyridinyl, pyridoimidazolyl, thiazolo[5,4-b]pyridin-2-yl, and thiazolo[5,4-d]pyrimidin-2-yl.

The term “heterocycle” or “heterocyclic,” as used herein, means a monocyclic heterocycle, a bicyclic heterocycle, or a tricyclic heterocycle. The term “heterocyclyl” is used herein to refer to a heterocycle when present as a substituent. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocyclyls include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, 2-oxo-3-piperidinyl, 2-oxoazepan-3-yl, oxadiazolinyl, oxadiazolidinyl, oxazolinyl, oxazolidinyl, oxetanyl, oxepanyl, oxocanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a 6-membered arene, or a monocyclic heterocycle fused to a monocyclic cycloalkane, or a monocyclic heterocycle fused to a monocyclic cycloalkene, or a monocyclic heterocycle fused to a monocyclic heterocycle, or a monocyclic heterocycle fused to a monocyclic heteroarene, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. The bicyclic heterocyclyl is attached to the parent molecular moiety at a non-aromatic ring atom (e.g., indolin-1-yl). Representative examples of bicyclic heterocyclyls include, but are not limited to, chroman-4-yl, 2,3-dihydrobenzofuran-2-yl, 2,3-dihydrobenzothien-2-yl, 1,2,3,4-tetrahydroisoquinolin-2-yl, 2-azaspiro[3.3]heptan-2-yl, 2-oxa-6-azaspiro[3.3]heptan-6-yl, azabicyclo[2.2.1]heptyl (including 2-azabicyclo[2.2.1]hept-2-yl), azabicyclo[3.1.0]hexanyl (including 3-azabicyclo[3.1.0]hexan-3-yl), 2,3-dihydro-1H-indol-1-yl, isoindolin-2-yl, octahydrocyclopenta[c]pyrrolyl, octahydropyrrolopyridinyl, tetrahydroisoquinolinyl, 7-oxabicyclo[2.2.1]heptanyl, hexahydro-2H-cyclopenta[b]furanyl, 2-oxaspiro[3.3]heptanyl, 3-oxaspiro[5.5]undecanyl, 6-oxaspiro[2.5]octan-1-yl, and 3-oxabicyclo[3.1.0]hexan-6-yl. Tricyclic heterocycles are exemplified by a bicyclic heterocycle fused to a 6-membered arene, or a bicyclic heterocycle fused to a monocyclic cycloalkane, or a bicyclic heterocycle fused to a monocyclic cycloalkene, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.13,7]decane), and oxa-adamantane (2-oxatricyclo[3.3.1.13,7]decane). The monocyclic, bicyclic, and tricyclic heterocyclyls are connected to the parent molecular moiety at a non-aromatic ring atom.

Where heterocyclic and heteroaromatic ring systems are defined to “contain” or as “containing” specified heteroatoms (e.g., 1-3 heteroatoms independently selected from the group consisting of O, N, and S), any ring atoms of the heterocyclic and heteroaromatic ring systems that are not one of the specified heteroatoms are carbon atoms.

The term “hydroxyl” or “hydroxy,” as used herein, means an —OH group.

The term “hydroxyalkyl,” as used herein, means at least one —OH group, is appended to the parent molecular moiety through an alkylene group, as defined herein.

The term “hydroxyfluoroalkyl,” as used herein, means at least one —OH group, is appended to the parent molecular moiety through a fluoroalkyl group, as defined herein.

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

The term “sulfonamide,” as used herein, means —S(O)₂NR^{d}— or —NR^{d}S(O)—, wherein R^{d} may be hydrogen, alkyl, cycloalkyl, aryl, heteroaryl, heterocycle, alkenyl, or heteroalkyl.

The term “substituents” refers to a group “substituted” on an aryl, alicyclic ring system, heteroaryl, heterocycle, amine, benzyl, phenyl or pyridinyl group at any atom of that group.

The term “substituted” refers to a group that may be further substituted with one or more non-hydrogen substituent groups. Substituents include, but are not limited to, halogen, ═O (oxo), ═S (thioxo), cyano, isocyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfanyl, sulfonyl, alkylsulfonyl, arylsulfonyl, aminosulfonyl, sulfinyl, —COOH, ketone, amide, carbamate, and acyl. For example, if a group is described as being “optionally substituted” (such as an alkyl, alkenyl, alkynyl, aryl, heteroaryl, cycloalkyl, heteroalkyl, heterocycle or other group such as an R group), it may have 0, 1, 2, 3, 4 or 5 substituents independently selected from halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

The term “ [symbol] ” designates a single bond (—) or a double bond (═).

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

The term “allosteric site” as used herein refers to a ligand binding site that is topographically distinct from the orthosteric binding site.

The term “modulator” as used herein refers to a molecular entity (e.g., but not limited to, a ligand and a compound of the invention) that modulates the activity of the target receptor protein.

The term “ligand” as used herein refers to a natural or synthetic molecular entity that is capable of associating or binding to a receptor to form a complex and mediate, prevent or modify a biological effect. Thus, the term “ligand” encompasses allosteric modulators, inhibitors, activators, agonists, antagonists, natural substrates and analogs of natural substrates.

The terms “natural ligand” and “endogenous ligand” as used herein are used interchangeably, and refer to a naturally occurring ligand, found in nature, which binds to a receptor.

The term “orthosteric site” as used herein refers to the primary binding site on a receptor that is recognized by the endogenous ligand or agonist for that receptor. For example, the orthosteric site in the mAChR M₄ receptor is the site that acetylcholine binds.

The term “mAChR M₄ receptor positive allosteric modulator” as used herein refers to any exogenously administered compound or agent that directly or indirectly augments the activity of the mAChR M₄ receptor in the presence or in the absence of acetylcholine, or another agonist, in an animal, in particular a mammal, for example a human. For example, a mAChR M₄ receptor positive allosteric modulator can increase the activity of the mAChR M₄ receptor in a cell in the presence of extracellular acetylcholine. The cell can be Chinese hamster ovary (CHO-K1) cells transfected with human mAChR M₄. The cell can be Chinese hamster ovary (CHO-K1) cells transfected with rat mAChR M₄ receptor. The cell can be Chinese hamster ovary (CHO-K1) cells transfected with a mammalian mAChR M₄. The term “mAChR M₄ receptor positive allosteric modulator” includes a compound that is a “mAChR M₄ receptor allosteric potentiator” or a “mAChR M₄ receptor allosteric agonist,” as well as a compound that has mixed activity comprising pharmacology of both an “mAChR M₄ receptor allosteric potentiator” and an “mAChR M₄ receptor allosteric agonist.” The term “mAChR M₄ receptor positive allosteric modulator also includes a compound that is a “mAChR M₄ receptor allosteric enhancer.”

The term “mAChR M₄ receptor allosteric potentiator” as used herein refers to any exogenously administered compound or agent that directly or indirectly augments the response produced by the endogenous ligand (such as acetylcholine) when the endogenous ligand binds to the orthosteric site of the mAChR M₄ receptor in an animal, in particular a mammal, for example a human. The mAChR M₄ receptor allosteric potentiator binds to a site other than the orthosteric site, that is, an allosteric site, and positively augments the response of the receptor to an agonist or the endogenous ligand. In some embodiments, an allosteric potentiator does not induce desensitization of the receptor, activity of a compound as an mAChR M₄ receptor allosteric potentiator provides advantages over the use of a pure mAChR M₄ receptor orthosteric agonist. Such advantages can include, for example, increased safety margin, higher tolerability, diminished potential for abuse, and reduced toxicity.

The term “mAChR M₄ receptor allosteric enhancer” as used herein refers to any exogenously administered compound or agent that directly or indirectly augments the response produced by the endogenous ligand (such as acetylcholine) in an animal, in particular a mammal, for example a human. In some embodiments, the allosteric enhancer increases the affinity of the natural ligand or agonist for the orthosteric site. In some embodiments, an allosteric enhancer increases the agonist efficacy. The mAChR M₄ receptor allosteric enhancer binds to a site other than the orthosteric site, that is, an allosteric site, and positively augments the response of the receptor to an agonist or the endogenous ligand. An allosteric enhancer has no effect on the receptor by itself and requires the presence of an agonist or the natural ligand to realize a receptor effect.

The term “mAChR M₄ receptor allosteric agonist” as used herein refers to any exogenously administered compound or agent that directly activates the activity of the mAChR M₄ receptor in the absence of the endogenous ligand (such as acetylcholine) in an animal, in particular a mammal, for example a human. The mAChR M₄ receptor allosteric agonist binds to a site that is distinct from the orthosteric acetylcholine site of the mAChR M₄ receptor. Because it does not require the presence of the endogenous ligand, activity of a compound as an mAChR M₄ receptor allosteric agonist provides advantages if cholinergic tone at a given synapse is low.

The term “mAChR M₄ receptor neutral allosteric ligand” as used herein refers to any exogenously administered compound or agent that binds to an allosteric site without affecting the binding or function of agonists or the natural ligand at the orthosteric site in an animal, in particular a mammal, for example a human. However, a neutral allosteric ligand can block the action of other allosteric modulators that act via the same site.

The term “mAChR M₄ receptor ago-allosteric ligand” as used herein refers to any exogenously administered compound or agent that functions both as an mAChR M₄ receptor agonist on its own and as an mAChR M₄ receptor allosteric enhancer in the presence of an agonist or the endogenous ligand.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. COMPOUNDS OF THE INVENTION

In one aspect, the invention provides a compound of formula (I), or a pharmaceutically acceptable salt thereof, wherein:

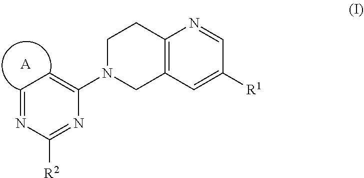

A is selected from a 5-membered heteroarene, a 5-membered heterocycle, and a 5-membered alicyclic ring system, wherein the heteroarene and heterocycle contain 1 to 2 heteroatoms independently selected from the group consisting of N, O, and S;
wherein the 5-membered heteroarene, the 5-membered heterocycle, and the 5-membered alicyclic ring system of A are unsubstituted or substituted with 1, 2, or 3 substituents independently selected from C₁₋₃ alkyl, C₁₋₄ fluoroalkyl, C₃₋₆ cycloalkyl, and halogen;
R¹ is selected from C₁₋₄ fluoroalkyl, C₃₋₆ cycloalkyl, phenyl, 5- or 6-membered heteroaryl, —NR^{a}R^{b} and -LR^{c};
L is CH₂ or O;
wherein R^{a} is a 5- or 6-membered heteroaryl or phenyl;
wherein R^{b} is hydrogen; or
wherein R^{a} and R^{b} together with the N to which they are attached form a heterocyclyl;
wherein R^{c} is 5- or 6-membered heteroaryl or phenyl;
wherein each phenyl, 5- or 6-membered heteroaryl, cycloalkyl, and heterocyclyl of R¹, R^{a}, or R^{c} is unsubstituted or substituted with 1, 2, or 3 substituents independently selected from C₁₋₄ alkyl, C₁₋₄ fluoroalkyl, —NR^{d}R^{e}, halogen, and C₃₋₆ cycloalkyl;
wherein R^{d} and R^{e} are independently selected from hydrogen and C₁₋₄ alkyl;
R² is selected from hydrogen, C₁₋₄ alkyl, halogen, —NR^{g}R^{h}, —ORⁱ and C₃₋₆ cycloalkyl;
wherein R^{g} is independently selected from C₁₋₄ alkyl, C₁₋₃ hydroxyalkyl, C₃₋₆ cycloalkyl, phenyl, and 5- or 6-membered heteroaryl;
wherein R^{h} is independently selected from hydrogen and C₁₋₄ alkyl; or
wherein R^{g} and R^{h} together with the N to which they are attached form a heterocyclyl;
wherein Rⁱ is C₁₋₄ alkyl;
wherein each 5- or 6-membered heteroaryl, heterocyclyl, phenyl, and C₃₋₆ cycloalkyl of R² is unsubstituted or substituted with 1, 2, or 3 substituents independently selected from —NR^{j}R^{k}, C₁₋₄ fluoroalkyl, hydroxyl, C₁₋₄ alkyl, halogen, C₃₋₆ cycloalkyl; and
wherein Rⁱ and R^{k} are independently selected from hydrogen and C₁₋₄ alkyl.

In another aspect, disclosed is a compound of formula (I), or a pharmaceutically acceptable salt thereof, wherein:
A is selected from a 5-membered heteroarene containing 1 to 2 heteroatoms independently selected from the group consisting of N, O, and S, and a 5-membered alicyclic ring system;
wherein the 5-membered heteroarene, and the 5-membered alicyclic ring system of A are unsubstituted or substituted with 1 or 2 substituents independently selected from C₁₋₃ alkyl, C₁₋₄ fluoroalkyl, C₃₋₆ cycloalkyl, and halogen;
R¹ is selected from C₁₋₄fluoroalkyl, C₃₋₆ cycloalkyl, 5- or 6-membered heteroaryl, —CH₂—R^{c}, —NR^{a}R^{b} and —OR^{c};
wherein R^{a} is a 5- or 6-membered heteroaryl;
wherein R^{b} is hydrogen; or
wherein R^{a} and R^{b} together with the N to which they are attached form a heterocyclyl;
wherein R^{c} is phenyl;
wherein each phenyl, 5- or 6-membered heteroaryl, and heterocyclyl of R¹, R^{a}, or R^{c} is unsubstituted or substituted with 1, 2, or 3 substituents independently selected from C₁₋₃ alkyl, C₁₋₄fluoroalkyl, —NR^{d}R^{e}, halogen, and C₃₋₆ cycloalkyl;
wherein R^{d} and R^{e} are independently selected from hydrogen and C₁₋₃ alkyl;
R² is selected from hydrogen, C₁₋₃ alkyl, halogen, —NR^{g}R^{h}, —ORⁱ and C₃₋₆ cycloalkyl;
wherein R^{g} is independently selected from C₁₋₃ alkyl, C₁₋₃ hydroxyalkyl, C₃₋₆ cycloalkyl, phenyl, and 5- or 6-membered heteroaryl;
wherein R^{h} is independently selected from hydrogen and C₁₋₃ alkyl; or
wherein R^{g} and R^{h} together with the N to which they are attached form a heterocyclyl;
wherein Rⁱ is C₁₋₃ alkyl;
wherein each 5- or 6-membered heteroaryl, heterocyclyl, phenyl, and C₃₋₆ cycloalkyl of R² is unsubstituted or substituted with 1, 2, or 3 substituents independently selected from —NR^{j}R^{k}, C₁₋₄fluoroalkyl, hydroxyl, C₁₋₃ alkyl, halogen, C₃₋₆ cycloalkyl; and
wherein Rⁱ and R^{k} are independently selected from hydrogen and C₁₋₃ alkyl.

In an embodiment, the 5-membered heteroarene of A is thieno, furo, isoxazolo, pyrazolo, or pyrrolo, wherein A is unsubstituted or substituted with 1 or 2 substituents independently selected from C₁₋₃ alkyl, C₁₋₄ fluoroalkyl, C₃₋₆ cycloalkyl, and halogen.

In an embodiment, the 5-membered heteroarene of A is selected from thieno unsubstituted or substituted with 1 or 2 substituents independently selected from C₁₋₃ alkyl, C₃₋₆ cycloalkyl and halogen; furo unsubstituted or substituted with 1 methyl; isoxazolo unsubstituted or substituted with 1 methyl; pyrazolo unsubstituted or substituted with 1 methyl; and pyrrolo unsubstituted or substituted with 1 methyl.

In an embodiment, the 5-membered alicyclic ring system of A is an unsubstituted 5-membered alicyclic ring system.

In an embodiment, the 5-membered heterocycle of A contains one heteroatom selected from oxygen.

In an embodiment, the 5-membered heteroarene of A is thieno unsubstituted or substituted with 1 or 2 methyl.

In an embodiment, the 5-membered heteroarene of A is furo substituted with 1 methyl.

In an embodiment, the 5-membered heteroarene of A is isoxazolo substituted with 1 methyl.

In an embodiment, the 5-membered heteroarene of A is pyrazolo substituted with 1 methyl.

In an embodiment, the 5-membered heteroarene of A is pyrrolo is substituted with 1 methyl.

In an embodiment, the C₁₋₄ fluoroalkyl of R¹ is trifluoromethyl.

In an embodiment, the 5- or 6-membered heteroaryl of R¹ is selected from isoxazolyl substituted with 2 methyl; pyridyl substituted with 1 methyl; thiazolyl substituted with 1 methyl; pyrazolyl substituted with 1 or 2 substituents independently selected from cyclopropyl and methyl.

In an embodiment, R^{a} and R^{b} of R¹ together with the N to which they are attached form a heterocyclyl, the heterocyclyl is a monocyclic or bicyclic heterocyclyl unsubstituted or substituted with 1 or 2 substituents independently selected from C₁₋₃ alkyl, halogen, and C₃₋₆ cycloalkyl. In a further embodiment, the monocyclic heterocyclyl is a morpholino unsubstituted or substituted with 1-2 methyl (e.g., 2-methylmorpholine such as (R)-2-methylmorpholine), and the bicyclic heterocyclyl is selected from an unsubstituted dihydropyrazolopyrimidinyl and an unsubstituted dihydropyridooxazinyl.

In an embodiment, the R^{a} is selected from pyridyl and pyrazolyl, each unsubstituted or substituted with 1 or 2 substituents; and R^{b} is hydrogen. In a further embodiment, the pyridyl and pyrazolyl is unsubstituted or substituted with 1 or 2 substituents independently selected from C₁₋₃ alkyl (e.g. methyl) and halogen (e.g. fluoro).

In an embodiment, the R^{c} is phenyl. In a further embodiment, the phenyl is unsubstituted or substituted with 1 or 2 substituents independently selected from C₁₋₃ alkyl (e.g. methyl) and halogen (e.g. fluoro).

In an embodiment, the R¹ is selected from trifluoromethyl; morpholin-4-yl unsubstituted or substituted with 1 methyl (e.g., (R)-2-methylmorpholin-4-yl); 6,7-dihydro-5H-pyrazolo[1,5-a]pyrimidin-4-yl; 2,3-dihydropyrido[2,3-b][1,4]oxazin-1-yl; isoxazolyl unsubstituted or substituted with 1 or 2 methyl; pyrazolyl unsubstituted or substituted with 1 or 2 substituents independently selected from methyl and cyclopropyl; pyridyl unsubstituted or substituted with 1 methyl; thiazolyl unsubstituted or substituted with 1 methyl; —CH₂-phenyl, wherein the phenyl is unsubstituted or substituted with 1 halogen; —NHR^{a}; and phenoxyl unsubstituted or substituted with 1 or 2 substituents independently selected from halogen and methyl; and R^{a} is selected from pyrazolyl unsubstituted or substituted with 1 methyl; and pyridinyl unsubstituted or substituted with 1 halogen.

In an embodiment, the R¹ is selected from

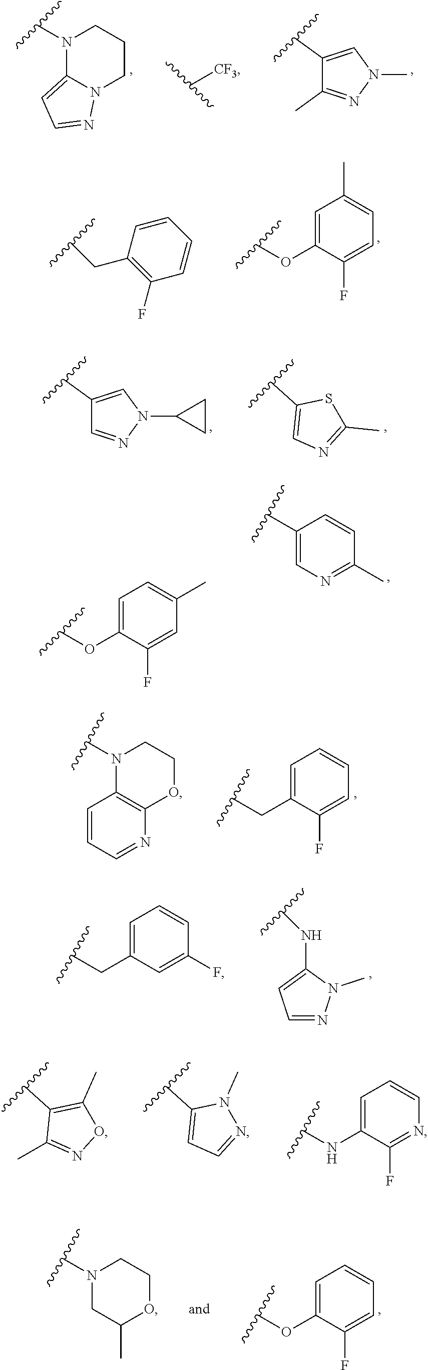

wherein [symbol] indicates the attachment point

In an embodiment, the R² is selected from hydrogen, chloro, cyclopropyl, and methyl.

In an embodiment, the R² is hydrogen.

In an embodiment, the R² is chloro.

In an embodiment, the R² is cyclopropyl.

In an embodiment, the R² is methyl.

In an embodiment of the invention, the compound is a compound of formula (Ia), or a pharmaceutically acceptable salt thereof, wherein:

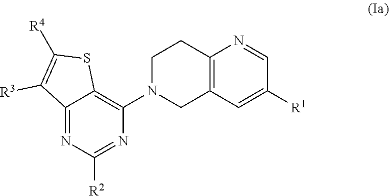

R³ is selected from hydrogen and C₁₋₃ alkyl; and
R⁴ is selected from hydrogen and C₁₋₃ alkyl.

In an embodiment, when the compound is a compound of formula (Ia), or a pharmaceutically acceptable salt thereof, the R³ is hydrogen.

In an embodiment, when the compound is a compound of formula (Ia), or a pharmaceutically acceptable salt thereof, the R³ is C₁₋₃ alkyl (e.g. methyl).

In an embodiment, when the compound is a compound of formula (Ia), or a pharmaceutically acceptable salt thereof, the R⁴ is hydrogen.

In an embodiment, when the compound is a compound of formula (Ia), or a pharmaceutically acceptable salt thereof, the R⁴ is C₁₋₃ alkyl (e.g. methyl).

In an embodiment of the invention, when the compound is a compound of formula (Ia), or a pharmaceutically acceptable salt thereof, the R¹ is C₁₋₄ fluoroalkyl. In a further embodiment, the C₁₋₄ fluoroalkyl of R¹ is trifluoromethyl.

In an embodiment, when the compound is a compound of formula (Ia), or a pharmaceutically acceptable salt thereof, the 5- or 6-membered heteroaryl of R¹ is selected from isoxazolyl, pyridyl, thiazolyl, and pyrazolyl, each unsubstituted or substituted with 1 or 2 substituents independently selected from C₁₋₃ alkyl, —NR^{d}R^{e}, halogen, and C₃₋₆ cycloalkyl.

In a further embodiment, when the compound is a compound of formula (Ia), or a pharmaceutically acceptable salt thereof, the 5- or 6-membered heteroaryl of R¹ is selected from pyridyl, pyrazolyl, and thiazolyl, each are unsubstituted or substituted with 1 or 2 substituents independently selected from C₁₋₃ alkyl, —NR^{d}R^{e}, halogen, and C₃₋₆ cycloalkyl. In a further embodiment, the pyridyl, pyrazolyl, and thiazolyl are unsubstituted or substituted with 1 or 2 substituents independently selected from methyl, cyclopropyl, and fluoro.

In an embodiment, when the compound is a compound of formula (Ia), or a pharmaceutically acceptable salt thereof, the R^{a} and R^{b} of R¹ together with the N to which they are attached form a heterocyclyl, the heterocyclyl is a bicyclic heterocyclyl unsubstituted or substituted with 1, 2, or 3 substituents independently selected from C₁₋₃ alkyl, halogen, and C₃₋₆ cycloalkyl. In a further embodiment, the bicyclic heterocyclyl is selected from an unsubstituted dihydropyrazolopyrimidinyl and an unsubstituted dihydropyridooxazinyl.

In an embodiment, when the compound is a compound of formula (Ia), or a pharmaceutically acceptable salt thereof, the R^{a} is selected from pyridyl and pyrazolyl, each are unsubstituted or substituted with 1 or 2 substituents, and R^{b} is hydrogen. In a further embodiment, when the compound is a compound of formula (Ia), the pyridyl and pyrazolyl are unsubstituted or substituted with 1 or 2 substituents independently selected from C₁₋₃ alkyl (e.g. methyl) and halogen (e.g. fluoro).

In an embodiment, when the compound is a compound of formula (Ia), or a pharmaceutically acceptable salt thereof, R^{c} is a phenyl unsubstituted or substituted with 1 or 2 substituents independently selected from C₁₋₃ alkyl and halogen.

In an embodiment, when the compound is a compound of formula (Ia), or a pharmaceutically acceptable salt thereof, R¹ is —OR^{c} and the R^{c} is phenyl. In a further embodiment, the phenyl is unsubstituted or substituted with 1 or 2 substituents independently selected from C₁₋₃ alkyl (e.g. methyl) and halogen (e.g. fluoro).

In an embodiment, when the compound is a compound of formula (Ia), or a pharmaceutically acceptable salt thereof, the R¹ is selected from trifluoromethyl; 6,7-dihydro-5H-pyrazolo[1,5-a]pyrimidin-4-yl; 2,3-dihydropyrido[2,3-b][1,4]oxazin-1-yl; isoxazolyl unsubstituted or substituted with 1 or 2 methyl; pyrazolyl unsubstituted or substituted with 1 or 2 substituents independently selected from methyl and cyclopropyl; pyridyl unsubstituted or substituted with 1 methyl; thiazolyl unsubstituted or substituted with 1 methyl; —CH₂-phenyl, wherein the phenyl is unsubstituted or substituted with 1 halogen; —NHR^{a}; and phenoxyl unsubstituted or substituted with 1 or 2 substituents independently selected from halogen and methyl; and R^{a} is selected from pyrazolyl unsubstituted or substituted with 1 methyl; and pyridinyl unsubstituted or substituted with 1 halogen.

In an embodiment, when the compound is a compound of formula (Ia), or a pharmaceutically acceptable salt thereof, the R² is hydrogen.

In an embodiment of the invention, the compound is a compound of formula (Ib), or a pharmaceutically acceptable salt thereof, wherein:

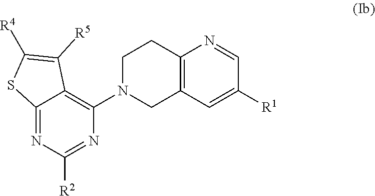

R⁴ is selected from hydrogen, halogen, and C₁₋₃ alkyl; and
R⁵ is selected from hydrogen, halogen, C₃₋₆ cycloalkyl and C₁₋₃ alkyl.

In an embodiment, when the compound is a compound of formula (Ib), or a pharmaceutically acceptable salt thereof, the R⁵ is hydrogen. In a further embodiment, R⁵ is deuterium.

In an embodiment, when the compound is a compound of formula (Ib), or a pharmaceutically acceptable salt thereof, the R⁵ is C₁₋₃ alkyl. In a further embodiment R⁵ is selected from isopropyl and methyl.

In an embodiment, when the compound is a compound of formula (Ib), or a pharmaceutically acceptable salt thereof, the R⁵ is cyclopropyl.

In an embodiment, when the compound is a compound of formula (Ib), or a pharmaceutically acceptable salt thereof, the R⁵ is halogen (e.g. bromo).

In an embodiment, when the compound is a compound of formula (Ib), or a pharmaceutically acceptable salt thereof, the R⁴ is halogen (e.g. bromo).

In an embodiment, when the compound is a compound of formula (Ib), or a pharmaceutically acceptable salt thereof, the R⁴ is hydrogen. In a further embodiment, the hydrogen of R⁴ is deuterium.

In an embodiment, when the compound is a compound of formula (Ib), or a pharmaceutically acceptable salt thereof, the R⁴ is C₁₋₃ alkyl (e.g. methyl).

In an embodiment, when the compound is a compound of formula (Ib), or a pharmaceutically acceptable salt thereof, both R⁴ and R⁵ are methyl.

In an embodiment of the invention, when the compound is a compound of formula (Ib), or a pharmaceutically acceptable salt thereof, the R¹ is C₁₋₄ fluoroalkyl. In a further embodiment, the C₁₋₄ fluoroalkyl of R¹ is trifluoromethyl.

In an embodiment, when the compound is a compound of formula (Ib), or a pharmaceutically acceptable salt thereof, the 5- or 6-membered heteroaryl of R¹ is selected from isoxazolyl, pyridyl, thiazolyl, and pyrazolyl, each are unsubstituted or substituted with 1 or 2 substituents independently selected from C₁₋₃ alkyl, —NR^{d}R^{e}, halogen, and C₃₋₆ cycloalkyl. In a further embodiment, isoxazolyl, pyridyl, pyrazolyl, and thiazolyl are unsubstituted or substituted with 1 or 2 substituents independently selected from methyl, cyclopropyl, and fluoro.

In an embodiment, when the compound is a compound of formula (Ib), or a pharmaceutically acceptable salt thereof, the R^{a} and R^{b} of R¹ together with the N to which they are attached form a heterocyclyl, the heterocyclyl is a bicyclic heterocyclyl unsubstituted or substituted with 1 or 2 substituents independently selected from C₁₋₃ alkyl, halogen, and C₃₋₆ cycloalkyl. In a further embodiment, the bicyclic heterocyclyl is selected from an unsubstituted dihydropyrazolopyrimidinyl and an unsubstituted dihydropyridooxazinyl.

In an embodiment, when the compound is a compound of formula (Ib), or a pharmaceutically acceptable salt thereof, the R^{a} is selected from pyridyl and pyrazolyl, each are unsubstituted or substituted with 1 or 2 substituents; and R^{b} is hydrogen. In a further embodiment, the pyridyl and pyrazolyl are unsubstituted or substituted with 1 or 2 substituents independently selected from C₁₋₃ alkyl (e.g. methyl) and halogen (e.g. fluoro).

In an embodiment, when the compound is a compound of formula (Ib), or a pharmaceutically acceptable salt thereof, R^{c} is a phenyl unsubstituted or substituted with 1 or 2 substituents independently selected from C₁₋₃ alkyl and halogen.

In an embodiment, when the compound is a compound of formula (Ib), or a pharmaceutically acceptable salt thereof, R¹ is —OR^{c} and the R^{c} is phenyl. In a further embodiment, the phenyl is unsubstituted or substituted with 1 or 2 substituents independently selected from C₁₋₃ alkyl (e.g. methyl) and halogen (e.g. fluoro).

In an embodiment, when the compound is a compound of formula (Ib), or a pharmaceutically acceptable salt thereof, the R¹ is —CH₂—R^{c} and the R^{c} is phenyl. In a further embodiment, the phenyl is unsubstituted or substituted with 1 or 2 substituents independently selected from methyl and fluoro.

In an embodiment, when the compound is a compound of formula (Ib), or a pharmaceutically acceptable salt thereof, the R¹ is selected from trifluoromethyl; 6,7-dihydro-5H-pyrazolo[1,5-a]pyrimidin-4-yl; 2,3-dihydropyrido[2,3-b][1,4]oxazin-1-yl; isoxazolyl unsubstituted or substituted with 1 or 2 methyl; pyrazolyl unsubstituted or substituted with 1 or 2 substituents independently selected from methyl and cyclopropyl, pyridyl unsubstituted or substituted with 1 methyl; thiazolyl unsubstituted or substituted with 1 methyl; —CH₂-phenyl, wherein the phenyl is unsubstituted or substituted with 1 halogen; —NHR^{a}; and phenoxyl unsubstituted or substituted with 1 or 2 substituents independently selected from halogen and methyl; and R^{a} is selected from pyrazolyl unsubstituted or substituted with 1 methyl; and pyridinyl unsubstituted or substituted with 1 halogen.

In an embodiment, when the compound is a compound of formula (Ib), or a pharmaceutically acceptable salt thereof, the R² is hydrogen.

In an embodiment, when the compound is a compound of formula (Ib), or a pharmaceutically acceptable salt thereof, the R² is methyl.

In an embodiment of the invention, the compound is a compound of formula (Ic), or a pharmaceutically acceptable salt thereof, wherein:

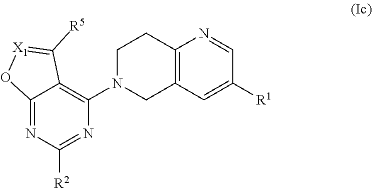

X₁ is selected from N and CR⁶,
wherein R⁶ is selected from hydrogen and C₁₋₃ alkyl; and
R⁵ is selected from hydrogen and C₁₋₃ alkyl.

In an embodiment, when the compound is a compound of formula (Ic), or a pharmaceutically acceptable salt thereof, the X₁ is N

In an embodiment, when the compound is a compound of formula (Ic), or a pharmaceutically acceptable salt thereof, the X₁ is CR⁶.

In an embodiment, when the compound is a compound of formula (Ic), or a pharmaceutically acceptable salt thereof, the R⁶ is C₁₋₃ alkyl (e.g. methyl).

In an embodiment, when the compound is a compound of formula (Ic), or a pharmaceutically acceptable salt thereof, the R⁵ is hydrogen.

In an embodiment, when the compound is a compound of formula (Ic), or a pharmaceutically acceptable salt thereof, the R⁵ is C₁₋₃ alkyl (e.g. methyl).

In an embodiment of the invention, when the compound is a compound of formula (Ic), or a pharmaceutically acceptable salt thereof, the R¹ is C₁₋₄ fluoroalkyl. In a further embodiment, the C₁₋₄ fluoroalkyl of R¹ is trifluoromethyl.

In an embodiment, when the compound is a compound of formula (Ic), or a pharmaceutically acceptable salt thereof, the 5- or 6-membered heteroaryl of R¹ is selected from isoxazolyl, pyridyl, thiazolyl, and pyrazolyl, each are unsubstituted or substituted with 1 or 2 substituents independently selected from C₁₋₃ alkyl, —NR^{d}R^{e}, halogen, and C₃₋₆ cycloalkyl. In a further embodiment, the pyridyl, pyrazolyl, and thiazolyl are unsubstituted or substituted with 1 or 2 substituents independently selected from methyl, cyclopropyl, and fluoro.

In an embodiment, when the compound is a compound of formula (Ic), or a pharmaceutically acceptable salt thereof, the R^{a} and R^{b} of R¹ together with the N to which they are attached form a heterocyclyl, the heterocyclyl is a bicyclic heterocyclyl unsubstituted or substituted with 1 or 2 substituents independently selected from C₁₋₃ alkyl, halogen, and C₃₋₆ cycloalkyl. In a further embodiment, the bicyclic heterocyclyl is selected from an unsubstituted dihydropyrazolopyrimidinyl and an unsubstituted dihydropyridooxazinyl.

In an embodiment, when the compound is a compound of formula (Ic), or a pharmaceutically acceptable salt thereof, the R^{a} is selected from pyridyl and pyrazolyl, each are unsubstituted or substituted with 1 or 2 substituents; and R^{b} is hydrogen. In a further embodiment, the pyridyl and pyrazolyl are unsubstituted or substituted with 1 or 2 substituents independently selected from a C₁₋₃ alkyl (e.g. methyl) and a halogen (e.g. fluoro).

In an embodiment, when the compound is a compound of formula (Ic), or a pharmaceutically acceptable salt thereof, R^{c} is a phenyl unsubstituted or substituted with 1 or 2 substituents independently selected from C₁₋₃ alkyl and halogen.

In an embodiment, when the compound is a compound of formula (Ic), or a pharmaceutically acceptable salt thereof, R¹ is —OR^{c} and the R^{c} is phenyl. In a further embodiment, the phenyl is unsubstituted or substituted with 1 or 2 substituents independently selected from C₁₋₃ alkyl (e.g. methyl) and halogen (e.g. fluoro).

In an embodiment, when the compound is a compound of formula (Ic), or a pharmaceutically acceptable salt thereof, the R¹ is —CH₂—R^{c} and the R^{c} is phenyl. In a further embodiment, the phenyl is unsubstituted or substituted with 1 or 2 substituents independently selected from a C₁₋₃ alkyl (e.g. methyl) and a halogen (e.g. fluoro).

In an embodiment, when the compound is a compound of formula (Ic), or a pharmaceutically acceptable salt thereof, the R¹ is selected from trifluoromethyl; 6,7-dihydro-5H-pyrazolo[1,5-a]pyrimidin-4-yl; 2,3-dihydropyrido[2,3-b][1,4]oxazin-1-yl; isoxazolyl unsubstituted or substituted with 1 or 2 methyl; pyrazolyl unsubstituted or substituted with 1 or 2 substituents independently selected from methyl and cyclopropyl; pyridyl unsubstituted or substituted with 1 methyl; thiazolyl unsubstituted or substituted with 1 methyl; —CH₂-phenyl, wherein the phenyl is unsubstituted or substituted with 1 halogen; —NHR^{a}; and phenoxyl unsubstituted or substituted with 1 or 2 substituents independently selected from a halogen and methyl; and R^{a} is selected from pyrazolyl unsubstituted or substituted with 1 methyl; and pyridinyl unsubstituted or substituted with 1 halogen.

In an embodiment, when the compound is a compound of formula (Ic), or a pharmaceutically acceptable salt thereof, the R² is hydrogen.

In an embodiment of the invention, the compound is a compound of formula (Id), or a pharmaceutically acceptable salt thereof, wherein:

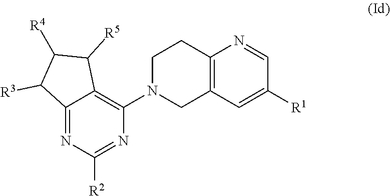

R³ is selected from hydrogen and C₁₋₃ alkyl;
R⁴ is selected from hydrogen and C₁₋₃ alkyl; and
R⁵ is selected from hydrogen and C₁₋₃ alkyl.

In an embodiment, when the compound is a compound of formula (Id), or a pharmaceutically acceptable salt thereof, the R³, R⁴, and R⁵ are hydrogen.

In an embodiment of the invention, when the compound is a compound of formula (Id), or a pharmaceutically acceptable salt thereof, the R¹ is a C₁₋₄ fluoroalkyl. In a further embodiment, the C₁₋₄ fluoroalkyl of R¹ is trifluoromethyl.

In an embodiment, when the compound is a compound of formula (Id), or a pharmaceutically acceptable salt thereof, the 5- or 6-membered heteroaryl of R¹ is selected from isoxazolyl, pyridyl, thiazolyl, and pyrazolyl, each are unsubstituted or substituted with 1 or 2 substituents independently selected from C₁₋₃ alkyl, —NR^{d}R^{e}, halogen, and C₃₋₆ cycloalkyl. In a further embodiment, the pyridyl, pyrazolyl, and thiazolyl are unsubstituted or substituted with 1 or 2 substituents independently selected from methyl, cyclopropyl, and fluoro.

In an embodiment, when the compound is a compound of formula (Id), or a pharmaceutically acceptable salt thereof, the R^{a} and R^{b} of R¹ together with the N to which they are attached form a heterocyclyl, the heterocyclyl is a bicyclic heterocyclyl unsubstituted or substituted with 1 or 2 substituents independently selected from C₁₋₃ alkyl, halogen, and C₃₋₆ cycloalkyl. In a further embodiment, the bicyclic heterocyclyl is selected from an unsubstituted dihydropyrazolopyrimidinyl and an unsubstituted dihydropyridooxazinyl.

In an embodiment, when the compound is a compound of formula (Id), or a pharmaceutically acceptable salt thereof, the R^{a} is selected from pyridyl and pyrazolyl, each are unsubstituted or substituted with 1 or 2 substituents, and R^{b} is hydrogen. In a further embodiment, the pyridyl and pyrazolyl are unsubstituted or substituted with 1 or 2 substituents independently selected from C₁₋₃ alkyl (e.g. methyl) and halogen (e.g. fluoro).

In an embodiment, when the compound is a compound of formula (Id), or a pharmaceutically acceptable salt thereof, R^{c} is a phenyl unsubstituted or substituted with 1 or 2 substituents independently selected from C₁₋₃ alkyl and halogen.

In an embodiment, when the compound is a compound of formula (Id), or a pharmaceutically acceptable salt thereof, R¹ is —OR^{c} and the R^{c} is phenyl. In a further embodiment, the phenyl is unsubstituted or substituted with 1 or 2 substituents independently selected from C₁₋₃ alkyl (e.g. methyl) and halogen (e.g. fluoro).

In an embodiment, when the compound is a compound of formula (Id), or a pharmaceutically acceptable salt thereof, the R¹ is —CH₂—R^{c} and the R^{c} is phenyl. In a further embodiment, the phenyl is unsubstituted or substituted with 1 or 2 substituents independently selected from C₁₋₃ alkyl (e.g. methyl) and halogen (e.g. fluoro).

In an embodiment, when the compound is a compound of formula (Id), or a pharmaceutically acceptable salt thereof, the R¹ is selected from trifluoromethyl; 6,7-dihydro-5H-pyrazolo[1,5-a]pyrimidin-4-yl; 2,3-dihydropyrido[2,3-b][1,4]oxazin-1-yl; isoxazolyl unsubstituted or substituted with 1 or 2 methyl; pyrazolyl unsubstituted or substituted with 1 or 2 substituents independently selected from methyl and cyclopropyl; pyridyl unsubstituted or substituted with 1 methyl; thiazolyl unsubstituted or substituted with 1 methyl; —CH₂-phenyl, wherein the phenyl is unsubstituted or substituted with 1 halogen; —NHR^{a}; and phenoxyl unsubstituted or substituted with 1 or 2 substituents independently selected from halogen and methyl; and R^{a} is selected from pyrazolyl unsubstituted or substituted with 1 methyl; and pyridinyl unsubstituted or substituted with 1 halogen.

In an embodiment, when the compound is a compound of formula (Id), or a pharmaceutically acceptable salt thereof, the R² is hydrogen.

In an embodiment, when the compound is a compound of formula (Id), or a pharmaceutically acceptable salt thereof, the R² is chloro.

In an embodiment, when the compound is a compound of formula (Id), or a pharmaceutically acceptable salt thereof, the R² is cyclopropyl.

In an embodiment, when the compound is a compound of formula (Id), or a pharmaceutically acceptable salt thereof, the R² is methyl.

In an embodiment of the invention, the compound is a compound of formula (Ie), or a pharmaceutically acceptable salt thereof, wherein:

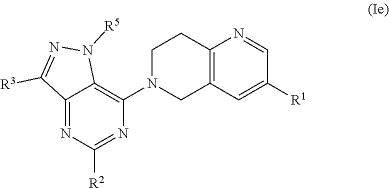

R³ is selected from hydrogen and C₁₋₃ alkyl; and
R⁵ is selected from hydrogen and C₁₋₃ alkyl.

In an embodiment, when the compound is a compound of formula (Ie), or a pharmaceutically acceptable salt thereof, R³ is hydrogen and R⁵ is C₁₋₃ alkyl. In an embodiment, R³ is hydrogen and R⁵ is methyl.

In an embodiment of the invention, when the compound is a compound of formula (Ie), or a pharmaceutically acceptable salt thereof, the R¹ is a C₁₋₄ fluoroalkyl. In a further embodiment, the C₁₋₄ fluoroalkyl of R¹ is trifluoromethyl.

In an embodiment, when the compound is a compound of formula (Ie), or a pharmaceutically acceptable salt thereof, the R² is hydrogen.

In an embodiment of the invention, the compound is a compound of formula (If), or a pharmaceutically acceptable salt thereof, wherein:

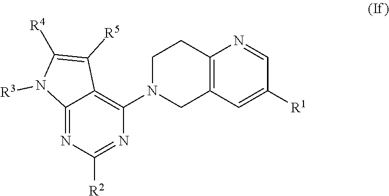

R³ is selected from hydrogen and C₁₋₃ alkyl;
R⁴ is selected from hydrogen and C₁₋₃ alkyl; and
R⁵ is selected from hydrogen and C₁₋₃ alkyl.

In an embodiment, when the compound is a compound of formula (If), or a pharmaceutically acceptable salt thereof, R³ is C₁₋₃ alkyl and R⁴ and R⁵ are hydrogen. In an embodiment, R³ is methyl; and R⁴ and R⁵ are hydrogen.

In an embodiment of the invention, when the compound is a compound of formula (If), or a pharmaceutically acceptable salt thereof, the R¹ is a C₁₋₄fluoroalkyl. In a further embodiment, the C₁₋₄ fluoroalkyl of R¹ is trifluoromethyl.

In an embodiment, when the compound is a compound of formula (If), or a pharmaceutically acceptable salt thereof, the R² is hydrogen.

In an embodiment of the invention, the compound is a compound of formula (Ig), or a pharmaceutically acceptable salt thereof.

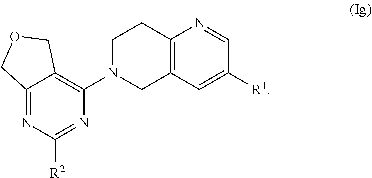

In an embodiment of the invention, when the compound is a compound of formula (Ig), or a pharmaceutically acceptable salt thereof, the R¹ is a C₁₋₄fluoroalkyl. In a further embodiment, the C₁₋₄ fluoroalkyl of R¹ is trifluoromethyl.

In an embodiment, when the compound is a compound of formula (Ig), or a pharmaceutically acceptable salt thereof, the R^{a} and R^{b} of R¹ together with the N to which they are attached form a heterocyclyl, the heterocyclyl is a monocyclic or bicyclic heterocyclyl unsubstituted or substituted with 1 or 2 substituents independently selected from C₁₋₃ alkyl, halogen, and C₃₋₆ cycloalkyl. In a further embodiment, the heterocyclyl is selected from a morpholino unsubstituted or substituted with 1-2 methyl (e.g., 2-methylmorpholine such as (R)-2-methylmorpholine), an unsubstituted dihydropyrazolopyrimidinyl, and an unsubstituted dihydropyridooxazinyl.

In an embodiment, when the compound is a compound of formula (Ig), or a pharmaceutically acceptable salt thereof, the R^{a} is selected from pyridyl and pyrazolyl, each are unsubstituted or substituted with 1 or 2 substituents, and R^{b} is hydrogen. In a further embodiment, the pyridyl and pyrazolyl are unsubstituted or substituted with 1 or 2 substituents independently selected from C₁₋₃ alkyl (e.g. methyl) and halogen (e.g. fluoro).

In an embodiment, when the compound is a compound of formula (Ig), or a pharmaceutically acceptable salt thereof, R^{c} is a phenyl unsubstituted or substituted with 1 or 2 substituents independently selected from C₁₋₃ alkyl and halogen.

In an embodiment, when the compound is a compound of formula (Ig), or a pharmaceutically acceptable salt thereof, R¹ is —OR^{c} and the R^{c} is phenyl. In a further embodiment, the phenyl is unsubstituted or substituted with 1 or 2 substituents independently selected from C₁₋₃ alkyl (e.g. methyl) and halogen (e.g. fluoro).

In an embodiment, when the compound is a compound of formula (Ig), or a pharmaceutically acceptable salt thereof, the R¹ is C₃₋₆cycloalkyl (e.g., cyclopropyl).

In an embodiment, when the compound is a compound of formula (Ig), or a pharmaceutically acceptable salt thereof, the R¹ is selected from trifluoromethyl; morpholin-4-yl unsubstituted or substituted with 1 methyl (e.g., (R)-2-methylmorpholin-4-yl); 6,7-dihydro-5H-pyrazolo[1,5-a]pyrimidin-4-yl; 2,3-dihydropyrido[2,3-b][1,4]oxazin-1-yl; cyclopropyl; —NHR^{a}; and phenoxyl unsubstituted or substituted with 1 or 2 substituents independently selected from halogen and methyl; and R^{a} is selected from pyrazolyl unsubstituted or substituted with 1 methyl; and pyridinyl unsubstituted or substituted with 1 halogen.

In an embodiment, when the compound is a compound of formula (Ig), or a pharmaceutically acceptable salt thereof, the R² is chloro.

In an embodiment of the invention, the compound is selected from:
5,6-dimethyl-4-[3-(trifluoromethyl)-7,8-dihydro-5H-1,6-naphthyridin-6-yl]thieno[2,3-d]pyrimidine;
2,5,6-trimethyl-4-[3-(trifluoromethyl)-7,8-dihydro-5H-1,6-naphthyridin-6-yl]thieno[2,3-d]pyrimidine;
5-methyl-4-[3-(trifluoromethyl)-7,8-dihydro-5H-1,6-naphthyridin-6-yl]furo[2,3-d]pyrimidine;
4-[3-(6,7-dihydro-5H-pyrazolo[1,5-a]pyrimidin-4-yl)-7,8-dihydro-5H-1,6-naphthyridin-6-yl]-5-methyl-furo[2,3-d]pyrimidine;
5-methyl-4-[3-(trifluoromethyl)-7,8-dihydro-5H-1,6-naphthyridin-6-yl]thieno[2,3-d]pyrimidine;
6-methyl-4-[3-(trifluoromethyl)-7,8-dihydro-5H-1,6-naphthyridin-6-yl]thieno[2,3-d]pyrimidine;
6-(2-chloro-6,7-dihydro-5H-cyclopenta[d]pyrimidin-4-yl)-3-(trifluoromethyl)-7,8-dihydro-5H-1,6-naphthyridine;
4-[3-(trifluoromethyl)-7,8-dihydro-5H-1,6-naphthyridin-6-yl]thieno[3,2-d]pyrimidine;
7-methyl-4-[3-(trifluoromethyl)-7,8-dihydro-5H-1,6-naphthyridin-6-yl]thieno[3,2-d]pyrimidine;
3-methyl-4-[3-(trifluoromethyl)-7,8-dihydro-5H-1,6-naphthyridin-6-yl]isoxazolo[5,4-d]pyrimidine;
6-(1-methylpyrazolo[4,3-d]pyrimidin-7-yl)-3-(trifluoromethyl)-7,8-dihydro-5H-1,6-naphthyridine;
4-[3-(1,3-dimethylpyrazol-4-yl)-7,8-dihydro-5H-1,6-naphthyridin-6-yl]-3-methyl-isoxazolo[5,4-d]pyrimidine;
4-[3-(1,3-dimethylpyrazol-4-yl)-7,8-dihydro-5H-1,6-naphthyridin-6-yl]-5-methyl-furo[2,3-d]pyrimidine;
4-[3-(6,7-dihydro-5H-pyrazolo[1,5-a]pyrimidin-4-yl)-7,8-dihydro-5H-1,6-naphthyridin-6-yl]-3-methyl-isoxazolo[5,4-d]pyrimidine;
4-[3-(1-cyclopropylpyrazol-4-yl)-7,8-dihydro-5H-1,6-naphthyridin-6-yl]-5-methyl-furo[2,3-d]pyrimidine;
4-[3-(1-cyclopropylpyrazol-4-yl)-7,8-dihydro-5H-1,6-naphthyridin-6-yl]-3-methyl-isoxazolo[5,4-d]pyrimidine;
6-(7-methylpyrrolo[2,3-d]pyrimidin-4-yl)-3-(trifluoromethyl)-7,8-dihydro-5H-1,6-naphthyridine;
4-[3-(1,3-dimethylpyrazol-4-yl)-7,8-dihydro-5H-1,6-naphthyridin-6-yl]-6-methyl-thieno[2,3-d]pyrimidine;
1-[6-(6-methylthieno[2,3-d]pyrimidin-4-yl)-7,8-dihydro-5H-1,6-naphthyridin-3-yl]-2,3-dihydropyrido[2,3-b][1,4]oxazine;
6-methyl-4-[3-(6-methyl-3-pyridyl)-7,8-dihydro-5H-1,6-naphthyridin-6-yl]thieno[2,3-d]pyrimidine;
7-methyl-4-[3-(6-methyl-3-pyridyl)-7,8-dihydro-5H-1,6-naphthyridin-6-yl]thieno[3,2-d]pyrimidine;
6-(2-cyclopropyl-6,7-dihydro-5H-cyclopenta[d]pyrimidin-4-yl)-3-(trifluoromethyl)-7,8-dihydro-5H-1,6-naphthyridine;
6-(2-methyl-6,7-dihydro-5H-cyclopenta[d]pyrimidin-4-yl)-3-(trifluoromethyl)-7,8-dihydro-5H-1,6-naphthyridine;
1-[6-(5-methylfuro[2,3-d]pyrimidin-4-yl)-7,8-dihydro-5H-1,6-naphthyridin-3-yl]-2,3-dihydropyrido[2,3-b][1,4]oxazine;
5-methyl-4-[3-(6-methyl-3-pyridyl)-7,8-dihydro-5H-1,6-naphthyridin-6-yl]furo[2,3-d]pyrimidine;
5-methyl-4-[3-(2-methylthiazol-5-yl)-7,8-dihydro-5H-1,6-naphthyridin-6-yl]furo[2,3-d]pyrimidine;
1-[6-(5-methylthieno[2,3-d]pyrimidin-4-yl)-7,8-dihydro-5H-1,6-naphthyridin-3-yl]-2,3-dihydropyrido[2,3-b][1,4]oxazine;
5-methyl-4-[3-(6-methyl-3-pyridyl)-7,8-dihydro-5H-1,6-naphthyridin-6-yl]thieno[2,3-d]pyrimidine;
5-methyl-4-[3-(2-methylthiazol-5-yl)-7,8-dihydro-5H-1,6-naphthyridin-6-yl]thieno[2,3-d]pyrimidine;
1-[6-(3-methylisoxazolo[5,4-d]pyrimidin-4-yl)-7,8-dihydro-5H-1,6-naphthyridin-3-yl]-2,3-dihydropyrido[2,3-b][1,4]oxazine;
3-methyl-4-[3-(6-methyl-3-pyridyl)-7,8-dihydro-5H-1,6-naphthyridin-6-yl]isoxazolo[5,4-d]pyrimidine;
3-methyl-4-[3-(2-methylthiazol-5-yl)-7,8-dihydro-5H-1,6-naphthyridin-6-yl]isoxazolo[5,4-d]pyrimidine;
4-[3-(2-fluoro-4-methyl-phenoxy)-7,8-dihydro-5H-1,6-naphthyridin-6-yl]-5-methyl-thieno[2,3-d]pyrimidine;
4-[3-(6,7-dihydro-5H-pyrazolo[1,5-a]pyrimidin-4-yl)-7,8-dihydro-5H-1,6-naphthyridin-6-yl]-6-methyl-thieno[2,3-d]pyrimidine;
4-[3-(1-cyclopropylpyrazol-4-yl)-7,8-dihydro-5H-1,6-naphthyridin-6-yl]-6-methyl-thieno[2,3-d]pyrimidine;
6-methyl-4-[3-(2-methylthiazol-5-yl)-7,8-dihydro-5H-1,6-naphthyridin-6-yl]thieno[2,3-d]pyrimidine;
4-[3-(6,7-dihydro-5H-pyrazolo[1,5-a]pyrimidin-4-yl)-7,8-dihydro-5H-1,6-naphthyridin-6-yl]-7-methyl-thieno[3,2-d]pyrimidine;
4-[3-(1-cyclopropylpyrazol-4-yl)-7,8-dihydro-5H-1,6-naphthyridin-6-yl]-7-methyl-thieno[3,2-d]pyrimidine;
4-[3-(1,3-dimethylpyrazol-4-yl)-7,8-dihydro-5H-1,6-naphthyridin-6-yl]-7-methyl-thieno[3,2-d]pyrimidine;
1-[6-(7-methylthieno[3,2-d]pyrimidin-4-yl)-7,8-dihydro-5H-1,6-naphthyridin-3-yl]-2,3-dihydropyrido[2,3-b][1,4]oxazine;
7-methyl-4-[3-(2-methylthiazol-5-yl)-7,8-dihydro-5H-1,6-naphthyridin-6-yl]thieno[3,2-d]pyrimidine;
4-[3-(6,7-dihydro-5H-pyrazolo[1,5-a]pyrimidin-4-yl)-7,8-dihydro-5H-1,6-naphthyridin-6-yl]-5-methyl-thieno[2,3-d]pyrimidine;
4-[3-(1-cyclopropylpyrazol-4-yl)-7,8-dihydro-5H-1,6-naphthyridin-6-yl]-5-methyl-thieno[2,3-d]pyrimidine;
4-[3-(2-fluoro-5-methyl-phenoxy)-7,8-dihydro-5H-1,6-naphthyridin-6-yl]thieno[3,2-d]pyrimidine;
4-[3-[(2-fluorophenyl)methyl]-7,8-dihydro-5H-1,6-naphthyridin-6-yl]-5-methyl-furo[2,3-d]pyrimidine;
4-[3-(1,3-dimethylpyrazol-4-yl)-7,8-dihydro-5H-1,6-naphthyridin-6-yl]-5-methyl-thieno[2,3-d]pyrimidine;
5-bromo-4-[3-(trifluoromethyl)-7,8-dihydro-5H-1,6-naphthyridin-6-yl]thieno[2,3-d]pyrimidine;
6-bromo-4-[3-(trifluoromethyl)-7,8-dihydro-5H-1,6-naphthyridin-6-yl]thieno[2,3-d]pyrimidine;
4-[3-(6,7-dihydro-5H-pyrazolo[1,5-a]pyrimidin-4-yl)-7,8-dihydro-5H-1,6-naphthyridin-6-yl]-6-isopropyl-thieno[2,3-d]pyrimidine;
4-[3-[(3-fluorophenyl)methyl]-7,8-dihydro-5H-1,6-naphthyridin-6-yl]-5-methyl-furo[2,3-d]pyrimidine;
4-[3-(2-fluorophenoxy)-7,8-dihydro-5H-1,6-naphthyridin-6-yl]-5-methyl-thieno[2,3-d]pyrimidine;
4-[3-(2-fluorophenoxy)-7,8-dihydro-5H-1,6-naphthyridin-6-yl]thieno[2,3-d]pyrimidine;
4-[3-(2-methylthiazol-5-yl)-7,8-dihydro-5H-1,6-naphthyridin-6-yl]thieno[2,3-d]pyrimidine;
N-(2-methylpyrazol-3-yl)-6-thieno[2,3-d]pyrimidin-4-yl-7,8-dihydro-5H-1,6-naphthyridin-3-amine;
4-[6-(6-isopropylthieno[2,3-d]pyrimidin-4-yl)-7,8-dihydro-5H-1,6-naphthyridin-3-yl]-3,5-dimethyl-isoxazole;
5-deuterio-4-[3-(trifluoromethyl)-7,8-dihydro-5H-1,6-naphthyridin-6-yl]thieno[2,3-d]pyrimidine;
4-[3-(2-fluorophenoxy)-7,8-dihydro-5H-1,6-naphthyridin-6-yl]-6-methyl-thieno[2,3-d]pyrimidine;
5-cyclopropyl-4-[3-(trifluoromethyl)-7,8-dihydro-5H-1,6-naphthyridin-6-yl]thieno[2,3-d]pyrimidine;
6-(5-methylfuro[2,3-d]pyrimidin-4-yl)-N-(2-methylpyrazol-3-yl)-7,8-dihydro-5H-1,6-naphthyridin-3-amine;
N-(2-fluoro-3-pyridyl)-6-(5-methylfuro[2,3-d]pyrimidin-4-yl)-7,8-dihydro-5H-1,6-naphthyridin-3-amine; and
5-methyl-4-[3-(2-methylpyrazol-3-yl)-7,8-dihydro-5H-1,6-naphthyridin-6-yl]furo[2,3-d]pyrimidine;
2-chloro-4-[3-(trifluoromethyl)-7,8-dihydro-5H-1,6-naphthyridin-6-yl]-5,7-dihydrofuro[3,4-d]pyrimidine;
1-[6-(2-chloro-5,7-dihydrofuro[3,4-d]pyrimidin-4-yl)-7,8-dihydro-5H-1,6-naphthyridin-3-yl]-2,3-dihydropyrido[2,3-b][1,4]oxazine;
2-chloro-4-(3-(6,7-dihydropyrazolo[1,5-a]pyrimidin-4(5H)-yl)-7,8-dihydro-1,6-naphthyridin-6(5H)-yl)-5,7-dihydrofuro[3,4-d]pyrimidine;
(R)-2-chloro-4-(3-(2-methylmorpholino)-7,8-dihydro-1,6-naphthyridin-6(5H)-yl)-5,7-dihydrofuro[3,4-d]pyrimidine;
2-chloro-4-(3-cyclopropyl-7,8-dihydro-1,6-naphthyridin-6(5H)-yl)-5,7-dihydrofuro[3,4-d]pyrimidine;
6-(2-chloro-5,7-dihydrofuro[3,4-d]pyrimidin-4-yl)-N-(1-methyl-1H-pyrazol-5-yl)-5,6,7,8-tetrahydro-1,6-naphthyridin-3-amine;
6-(2-chloro-5,7-dihydrofuro[3,4-d]pyrimidin-4-yl)-N-(2-fluoropyridin-3-yl)-5,6,7,8-tetrahydro-1,6-naphthyridin-3-amine; and
2-chloro-4-(3-(2-fluorophenoxy)-7,8-dihydro-1,6-naphthyridin-6(5H)-yl)-5,7-dihydrofuro[3,4-d]pyrimidine, or a pharmaceutically acceptable salt thereof.

In the following, numbered embodiments of the invention are disclosed. The first embodiment is denoted E1, subsequent embodiments are denoted E2, E3, and so forth.

E1. A compound of formula (I), or a pharmaceutically acceptable salt thereof, wherein:

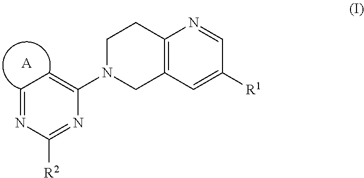

A is selected from a 5-membered heteroarene, a 5-membered heterocycle, and a 5-membered alicyclic ring system, wherein the heteroarene and heterocycle contain 1 to 2 heteroatoms independently selected from the group consisting of N, O, and S;
wherein the 5-membered heteroarene, the 5-membered heterocycle, and the 5-membered alicyclic ring system of A are unsubstituted or substituted with 1, 2, or 3 substituents independently selected from C₁₋₃ alkyl, C₁₋₄ fluoroalkyl, C₃₋₆ cycloalkyl, and halogen;
R¹ is selected from C₁₋₄ fluoroalkyl, C₃₋₆ cycloalkyl, phenyl, 5- or 6-membered heteroaryl, —NR^{a}R^{b} and -LR^{c};
L is CH₂ or O;
wherein R^{a} is a 5- or 6-membered heteroaryl or phenyl;
wherein R^{b} is hydrogen; or
wherein R^{a} and R^{b} together with the N to which they are attached form a heterocyclyl;
wherein R^{c} is 5- or 6-membered heteroaryl or phenyl;
wherein each phenyl, 5- or 6-membered heteroaryl, cycloalkyl, and heterocyclyl of R¹, R^{a}, or R^{c} is unsubstituted or substituted with 1, 2, or 3 substituents independently selected from C₁₋₄ alkyl, C₁₋₄ fluoroalkyl, —NR^{d}R^{e}, halogen, and C₃₋₆ cycloalkyl;
wherein R^{d} and R^{e} are independently selected from hydrogen and C₁₋₄ alkyl;
R² is selected from hydrogen, C₁₋₄ alkyl, halogen, —NR^{g}R^{h}, —ORⁱ and C₃₋₆ cycloalkyl;
wherein R^{g} is independently selected from C₁₋₄ alkyl, C₁₋₃ hydroxyalkyl, C₃₋₆ cycloalkyl, phenyl, and 5- or 6-membered heteroaryl;
wherein R^{h} is independently selected from hydrogen and C₁₋₄ alkyl; or
wherein R^{g} and R^{h} together with the N to which they are attached form a heterocyclyl;
wherein Rⁱ is C₁₋₄ alkyl;
wherein each 5- or 6-membered heteroaryl, heterocyclyl, phenyl, and C₃₋₆ cycloalkyl of R² is unsubstituted or substituted with 1, 2, or 3 substituents independently selected from —NR^{j}R^{k}, C₁₋₄ fluoroalkyl, hydroxyl, C₁₋₄ alkyl, halogen, C₃₋₆ cycloalkyl; and
wherein Rⁱ and R^{k} are independently selected from hydrogen and C₁₋₄ alkyl.

E2. A compound of E1, or a pharmaceutically acceptable salt thereof, wherein:
A is selected from a 5-membered heteroarene containing 1 to 2 heteroatoms independently selected from the group consisting of N, O, and S, and a 5-membered alicyclic ring system;
wherein the 5-membered heteroarene, and the 5-membered alicyclic ring system of A are unsubstituted or substituted with 1 or 2 substituents independently selected from C₁₋₃ alkyl, C₁₋₄ fluoroalkyl, C₃₋₆ cycloalkyl, and halogen;
R¹ is selected from C₁₋₄ fluoroalkyl, C₃₋₆ cycloalkyl, 5- or 6-membered heteroaryl, —CH₂—R^{c}, —NR^{a}R^{b} and —OR^{c};
wherein R^{a} is a 5- or 6-membered heteroaryl;
wherein R^{b} is hydrogen; or
wherein R^{a} and R^{b} together with the N to which they are attached form a heterocyclyl;
wherein R^{c} is phenyl;
wherein each phenyl, 5- or 6-membered heteroaryl, and heterocyclyl of R¹, R^{a}, or R^{c} are unsubstituted or substituted with 1, 2, or 3 substituents independently selected from C₁₋₃ alkyl, C₁₋₄ fluoroalkyl, —NR^{d}R^{e}, halogen, and C₃₋₆ cycloalkyl;
wherein R^{d} and R^{e} are independently selected from hydrogen and C₁₋₃ alkyl;
R² is selected from hydrogen, C₁₋₃ alkyl, halogen, —NR^{g}R^{h}, —ORⁱ and C₃₋₆ cycloalkyl;
wherein R^{j} is independently selected from C₁₋₃ alkyl, C₁₋₃ hydroxyalkyl, C₃₋₆ cycloalkyl, phenyl, and 5- or 6-membered heteroaryl;
wherein R^{h} is independently selected from hydrogen and C₁₋₃ alkyl; or
wherein R^{g} and R^{h} together with the N to which they are attached form a heterocyclyl;
wherein Rⁱ is C₁₋₃ alkyl;
wherein each 5- or 6-membered heteroaryl, heterocyclyl, phenyl, and C₃₋₆ cycloalkyl of R² are unsubstituted or substituted with 1, 2, or 3 substituents independently selected from —NR^{j}R^{k}, C₁₋₄ fluoroalkyl, hydroxyl, C₁₋₃ alkyl, halogen, C₃₋₆ cycloalkyl; and
wherein Rⁱ and R^{k} are independently selected from hydrogen and C₁₋₃ alkyl.

E3. A compound according to E1 or E2, or a pharmaceutically acceptable salt thereof, wherein the 5-membered heteroarene of A is selected from thieno, furo, isoxazolo, pyrazolo, and pyrrolo, each being unsubstituted or substituted with 1 or 2 substituents independently selected from C₁₋₃ alkyl, C₃₋₆ cycloalkyl, and halogen.

E4. A compound according to any of E1 to E3, or a pharmaceutically acceptable salt thereof, wherein the 5-membered alicyclic ring system of A is an unsubstituted 5-membered alicyclic ring system.

E5. A compound according to any of E1 to E4, or a pharmaceutically acceptable salt thereof, wherein the 5-membered heterocycle of A contains one heteroatom selected from oxygen.

E6. A compound according to any of E1 to E5, or a pharmaceutically acceptable salt thereof, wherein A is selected from thieno unsubstituted or substituted with 1 or 2 substituents independently selected from C₁₋₃ alkyl, C₃₋₆ cycloalkyl, and halogen; furo unsubstituted or substituted with 1 methyl; isoxazolo unsubstituted or substituted with 1 methyl; pyrazolo unsubstituted or substituted with 1 methyl; and pyrrolo unsubstituted or substituted with 1 methyl.

E7. A compound according to any of E1 to E6 having the formula (Ia), or a pharmaceutically acceptable salt thereof, wherein:

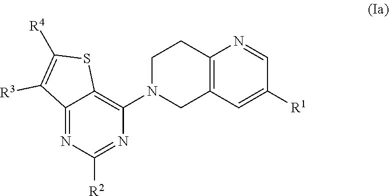

R³ is selected from hydrogen and C₁₋₃ alkyl; and
R⁴ is selected from hydrogen, halogen and C₁₋₃ alkyl.

E8. A compound according to E7, or a pharmaceutically acceptable salt thereof, wherein R³ is methyl.

E9. A compound according to E7, or a pharmaceutically acceptable salt thereof, wherein R³ is hydrogen.

E10. A compound according to any of E7-E9, or a pharmaceutically acceptable salt thereof, wherein R⁴ is methyl.

E11. A compound according to any of E7-E9, or a pharmaceutically acceptable salt thereof, wherein R⁴ is hydrogen.

E12. A compound according to any of E7-E9, or a pharmaceutically acceptable salt thereof, wherein R⁴ is bromo.

E13. A compound according to any of E1-E6 having the formula (Ib), or a pharmaceutically acceptable salt thereof, wherein:

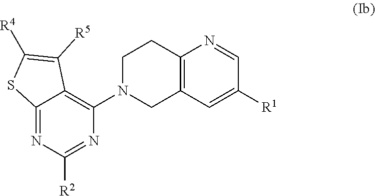

R⁴ is selected from hydrogen, halogen, and C₁₋₃ alkyl; and
R⁵ is selected from hydrogen, halogen, C₃₋₆ cycloalkyl and C₁₋₃ alkyl.

E14. A compound according to E13, or a pharmaceutically acceptable salt thereof, wherein R⁴ is hydrogen.

E15. A compound according to E13, or a pharmaceutically acceptable salt thereof, wherein R⁴ is bromo.

E16. A compound according to E13, or a pharmaceutically acceptable salt thereof, wherein R⁴ is methyl.

E17. A compound according to any of E13-E16, or a pharmaceutically acceptable salt thereof, wherein R⁵ is hydrogen.

E18. A compound according to any of E13-E16, or a pharmaceutically acceptable salt thereof, wherein R⁵ is bromo.

E19. A compound according to any of E13-E16, or a pharmaceutically acceptable salt thereof, wherein R⁵ is cyclopropyl.

E20. A compound according to any of E13-E16, or a pharmaceutically acceptable salt thereof, wherein R⁵ is methyl.

E21. A compound according to any of E1 to E6 having the formula (Ic), or a pharmaceutically acceptable salt thereof, wherein:

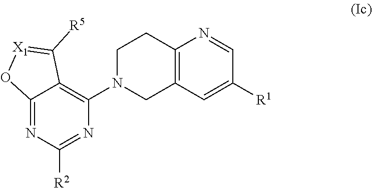

X₁ is selected from N and CR⁶,
wherein R⁶ is selected from hydrogen and C₁₋₃ alkyl; and
R⁵ is selected from hydrogen and C₁₋₃ alkyl.

E22. A compound according to E21, or a pharmaceutically acceptable salt thereof, wherein X₁ is N.

E23. A compound according to E21, or a pharmaceutically acceptable salt thereof, wherein X₁ is CR⁶.

E24. A compound according to E23, or a pharmaceutically acceptable salt thereof, wherein X₁ is CH.

E25. A compound according to any of E21 to E24, or a pharmaceutically acceptable salt thereof, wherein R⁵ is methyl.

E26. A compound according to any of E1 to E5 having the formula (Id), or a pharmaceutically acceptable salt thereof, wherein:

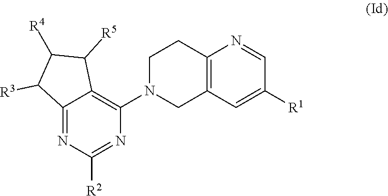

R³ is selected from hydrogen and C₁₋₃ alkyl;
R⁴ is selected from hydrogen and C₁₋₃ alkyl; and
R⁵ is selected from hydrogen and C₁₋₃ alkyl.

E27. A compound according to E26, or a pharmaceutically acceptable salt thereof, wherein R³, R⁴, R⁵ are hydrogen.

E28. A compound according to any of E1 to E6 having the formula (Ie), or a pharmaceutically acceptable salt thereof, wherein:

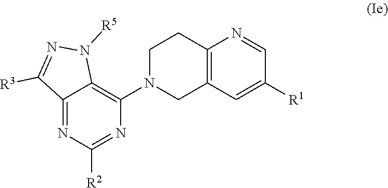

R³ is selected from hydrogen and C₁₋₃ alkyl; and
R⁵ is selected from hydrogen and C₁₋₃ alkyl.

E29. A compound according to E28, or a pharmaceutically acceptable salt thereof, wherein R³ is hydrogen and R⁵ is methyl.

E30. A compound according to any of E1 to E6 having the formula (If), or a pharmaceutically acceptable salt thereof, wherein:

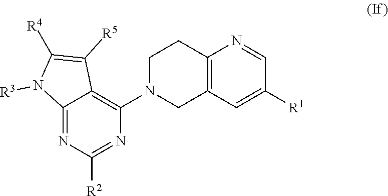

R³ is selected from hydrogen and C₁₋₃ alkyl;
R⁴ is selected from hydrogen and C₁₋₃ alkyl; and
R⁵ is selected from hydrogen and C₁₋₃ alkyl.

E31. A compound according to E30, or a pharmaceutically acceptable salt thereof, wherein R³ is methyl; and R⁴ and R⁵ are hydrogen.

E32. A compound according to any of E1 to E5 having the formula (Ig), or a pharmaceutically acceptable salt thereof

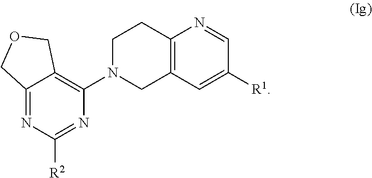

E33. A compound according to any of E1 to E32, or a pharmaceutically acceptable salt thereof, wherein the C₁₋₄fluoroalkyl of R¹ is trifluoromethyl.

E34. A compound according to any of E1-E32, or a pharmaceutically acceptable salt thereof, wherein the unsubstituted or substituted 5- or 6-membered heteroaryl of R¹ contains 1-3 heteroatoms independently selected from the group consisting of O, N, and S.

E34.1. A compound according to E34, or a pharmaceutically acceptable salt thereof, wherein the unsubstituted or substituted 5- or 6-membered heteroaryl of R¹ is selected from unsubstituted or substituted isoxazolyl, pyridyl, thiazolyl, and pyrazolyl.

E34.2. A compound according to any of E1-E32 or E34-E34.1, or a pharmaceutically acceptable salt thereof, wherein the unsubstituted or substituted 5- or 6-membered heteroaryl of R¹ is unsubstituted or substituted with 1 or 2 substituents independently selected from a C₁₋₃ alkyl, a halogen, and a C₃₋₆ cycloalkyl.

E34.3. A compound according to any of E1-E32 or E34 to E34.2, or a pharmaceutically acceptable salt thereof, wherein R¹ is the unsubstituted or substituted 5- or 6-membered heteroaryl.

E34.4. A compound according to E34.3, or a pharmaceutically acceptable salt thereof, wherein R¹ is

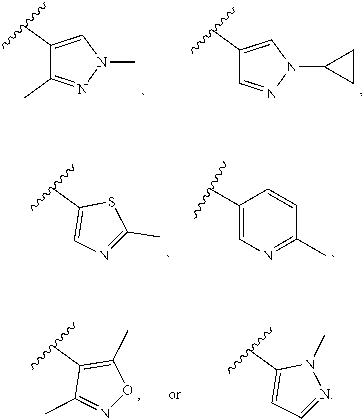

E35. A compound according to any of E1 to E32 or E34-E34.2, or a pharmaceutically acceptable salt thereof, wherein the R^{a} and R^{b} of R¹ together with the N to which they are attached form the unsubstituted or substituted heterocyclyl.

E35.1. A compound according to any of E1 to E32, E34-E34.2, or E35, or a pharmaceutically acceptable salt thereof, wherein the unsubstituted or substituted heterocyclyl formed by R^{a} and R^{b} of R¹ together with the N to which they are attached is an unsubstituted or substituted 4- to 12-membered heterocyclyl containing 1-3 heteroatoms independently selected from the group consisting of O, N, and S.

E35.2. A compound according to any of E1 to E32, E34 to E34.2, or E35 to E35.1, or a pharmaceutically acceptable salt thereof, wherein the unsubstituted or substituted heterocyclyl formed by R^{a} and R^{b} of R¹ together with the N to which they are attached is an unsubstituted or substituted monocyclic or bicyclic heterocyclyl.

E35.3. A compound according to any of E1 to E32, E34 to E34.2, or E35 to E35.2, or a pharmaceutically acceptable salt thereof, wherein the unsubstituted or substituted heterocyclyl formed by R^{a} and R^{b} of R¹ together with the N to which they are attached is unsubstituted or substituted with 1 or 2 substituents independently selected from C₁₋₃ alkyl, halogen, and C₃₋₆ cycloalkyl.

E35.4. A compound according to any of E35 to E35.3, or a pharmaceutically acceptable salt thereof, wherein R¹ is —NR^{a}R^{b}.

E36. A compound according to any of E35.2 to E35.4, or a pharmaceutically acceptable salt thereof, wherein the monocyclic heterocyclyl is a morpholino unsubstituted or substituted with 1-2 methyl, and the bicyclic heterocyclyl is selected from dihydropyrazolopyrimidinyl and dihydropyridooxazinyl, each are unsubstituted.

E36.1. A compound according to E36, or a pharmaceutically acceptable salt thereof, wherein R¹ is

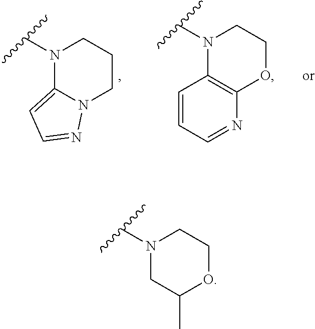

E37. A compound according to any of E1 to E32 or E34 to E34.2, or a pharmaceutically acceptable salt thereof, wherein R^{a} is the unsubstituted or substituted 5- or 6-membered heteroaryl.

E37.1. A compound according to any of E1 to E32, E34 to E34.2, or E37, or a pharmaceutically acceptable salt thereof, wherein the unsubstituted or substituted 5- or 6-membered heteroaryl of R^{a} contains 1-3 heteroatoms independently selected from the group consisting of O, N, and S.

E37.2. A compound according to E37.1, or a pharmaceutically acceptable salt thereof, wherein the unsubstituted or substituted 5- or 6-membered heteroaryl of R^{a} is selected from unsubstituted or substituted pyridyl and pyrazolyl.

E37.3. A compound according to any of E1 to E32, E34 to E34.2, or E37 to E37.2, or a pharmaceutically acceptable salt thereof, wherein the unsubstituted or substituted 5- or 6-membered heteroaryl of R^{a} is unsubstituted or substituted with 1 or 2 substituents independently selected from C₁₋₃ alkyl, halogen, and C₃₋₆ cycloalkyl.

E37.4. A compound according to any of E1 to E32, E34 to E34.2, or E37 to E37.3, or a pharmaceutically acceptable salt thereof, wherein R^{b} is hydrogen.

E37.5. A compound according to any of E37 to E37.4, or a pharmaceutically acceptable salt thereof, wherein R¹ is —NR^{a}R^{b}.

E37.6. A compound according to E37.5, or a pharmaceutically acceptable salt thereof, wherein R¹ is

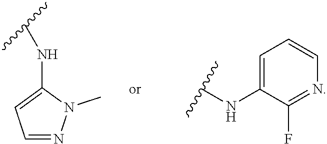

E38. A compound according to any of E1 to E32, E34 to E34.2, E35 to E35.3, E36, or E37 to E37.4, or a pharmaceutically acceptable salt thereof, wherein R^{c} is a phenyl unsubstituted or substituted with 1 or 2 substituents independently selected from C₁₋₃ alkyl and halogen.

E38.1. A compound according to any of E1 to E32, E34 to E34.2, E35 to E35.3, E36, E37 to E37.4, or E38, or a pharmaceutically acceptable salt thereof, wherein L is CH₂.

E38.2. A compound according to any of E1 to E32, E34 to E34.2, E35 to E35.3, E36, E37 to E37.4, or E38, or a pharmaceutically acceptable salt thereof, wherein L is O.

E38.3. A compound according to any of E1 to E32, E34 to E34.2, E35 to E35.3, E36, E37 to E37.4, or E38 to E38.2, or a pharmaceutically acceptable salt thereof, wherein R¹ is -LR^{c}.

E38.4. A compound according to E38.3, or a pharmaceutically acceptable salt thereof, wherein R¹ is

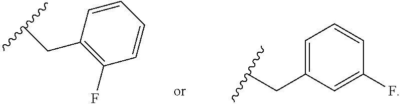 [SMILES: *Cc1ccccc1F ; *Cc1cccc(F)c1]

E38.5. A compound according to E38.3, or a pharmaceutically acceptable salt thereof, wherein R¹ is

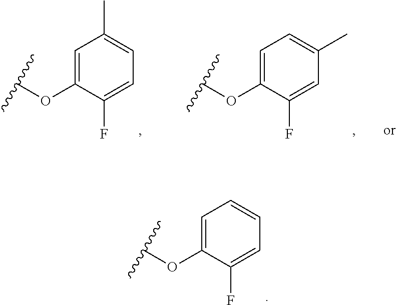

E39. A compound according to any of E1 to E32, or a pharmaceutically acceptable salt thereof, wherein
R¹ is selected from trifluoromethyl; morpholin-4-yl unsubstituted or substituted with 1 methyl; 6,7-dihydro-5H-pyrazolo[1,5-a]pyrimidin-4-yl; 2,3-dihydropyrido[2,3-b][1,4]oxazin-1-yl; isoxazolyl unsubstituted or substituted with 1 or 2 methyl; pyrazolyl unsubstituted or substituted with 1 or 2 substituents independently selected from methyl and cyclopropyl; pyridyl unsubstituted or substituted with 1 methyl; thiazolyl unsubstituted or substituted with 1 methyl; —CH₂-phenyl, wherein the phenyl is unsubstituted or substituted with 1 halogen; —NR^{a}; and phenoxyl unsubstituted or substituted with 1 or 2 substituents independently selected from halogen and methyl; and
R^{a} is selected from pyrazolyl unsubstituted or substituted with 1 methyl; and pyridinyl unsubstituted or substituted with 1 halogen.

E40. A compound according to any of E1 to E32, or a pharmaceutically acceptable salt thereof, wherein R¹ is selected from

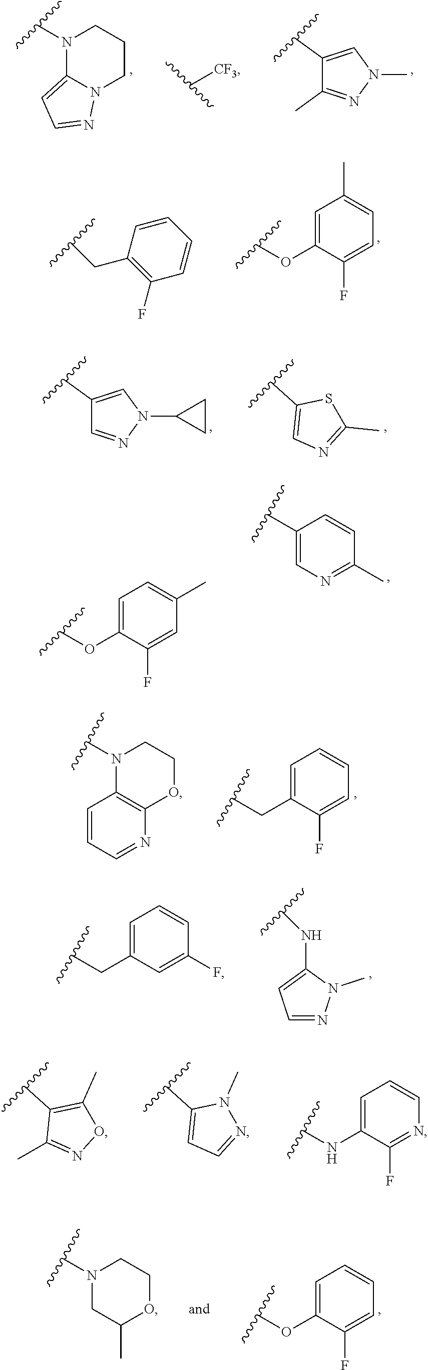

wherein

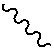

indicates the attachment point

E41. A compound according to any of E1 to E40, or a pharmaceutically acceptable salt thereof, wherein R² is selected from hydrogen, chloro, cyclopropyl, and methyl.

E42. A compound according to E41, or a pharmaceutically acceptable salt thereof wherein R² is hydrogen.

E43. A compound according to E41, or a pharmaceutically acceptable salt thereof wherein R² is chloro.

E44. A compound according to E41, or a pharmaceutically acceptable salt thereof, wherein R² is cyclopropyl.

E45. A compound according to E41, or a pharmaceutically acceptable salt thereof, wherein R² is methyl.

E46. A compound of E1, selected from:
5,6-dimethyl-4-[3-(trifluoromethyl)-7,8-dihydro-5H-1,6-naphthyridin-6-yl]thieno[2,3-d]pyrimidine;
2,5,6-trimethyl-4-[3-(trifluoromethyl)-7,8-dihydro-5H-1,6-naphthyridin-6-yl]thieno[2,3-d]pyrimidine;
5-methyl-4-[3-(trifluoromethyl)-7,8-dihydro-5H-1,6-naphthyridin-6-yl]furo[2,3-d]pyrimidine;
4-[3-(6,7-dihydro-5H-pyrazolo[1,5-a]pyrimidin-4-yl)-7,8-dihydro-5H-1,6-naphthyridin-6-yl]-5-methyl-furo[2,3-d]pyrimidine;
5-methyl-4-[3-(trifluoromethyl)-7,8-dihydro-5H-1,6-naphthyridin-6-yl]thieno[2,3-d]pyrimidine;
6-methyl-4-[3-(trifluoromethyl)-7,8-dihydro-5H-1,6-naphthyridin-6-yl]thieno[2,3-d]pyrimidine;
6-(2-chloro-6,7-dihydro-5H-cyclopenta[d]pyrimidin-4-yl)-3-(trifluoromethyl)-7,8-dihydro-5H-1,6-naphthyridine;
4-[3-(trifluoromethyl)-7,8-dihydro-5H-1,6-naphthyridin-6-yl]thieno[3,2-d]pyrimidine;
7-methyl-4-[3-(trifluoromethyl)-7,8-dihydro-5H-1,6-naphthyridin-6-yl]thieno[3,2-d]pyrimidine;
3-methyl-4-[3-(trifluoromethyl)-7,8-dihydro-5H-1,6-naphthyridin-6-yl]isoxazolo[5,4-d]pyrimidine;
6-(1-methylpyrazolo[4,3-d]pyrimidin-7-yl)-3-(trifluoromethyl)-7,8-dihydro-5H-1,6-naphthyridine;
4-[3-(1,3-dimethylpyrazol-4-yl)-7,8-dihydro-5H-1,6-naphthyridin-6-yl]-3-methyl-isoxazolo[5,4-d]pyrimidine;
4-[3-(1,3-dimethylpyrazol-4-yl)-7,8-dihydro-5H-1,6-naphthyridin-6-yl]-5-methyl-furo[2,3-d]pyrimidine;
4-[3-(6,7-dihydro-5H-pyrazolo[1,5-a]pyrimidin-4-yl)-7,8-dihydro-5H-1,6-naphthyridin-6-yl]-3-methyl-isoxazolo[5,4-d]pyrimidine;
4-[3-(1-cyclopropylpyrazol-4-yl)-7,8-dihydro-5H-1,6-naphthyridin-6-yl]-5-methyl-furo[2,3-d]pyrimidine;
4-[3-(1-cyclopropylpyrazol-4-yl)-7,8-dihydro-5H-1,6-naphthyridin-6-yl]-3-methyl-isoxazolo[5,4-d]pyrimidine;
6-(7-methylpyrrolo[2,3-d]pyrimidin-4-yl)-3-(trifluoromethyl)-7,8-dihydro-5H-1,6-naphthyridine;
4-[3-(1,3-dimethylpyrazol-4-yl)-7,8-dihydro-5H-1,6-naphthyridin-6-yl]-6-methyl-thieno[2,3-d]pyrimidine;
1-[6-(6-methylthieno[2,3-d]pyrimidin-4-yl)-7,8-dihydro-5H-1,6-naphthyridin-3-yl]-2,3-dihydropyrido[2,3-b][1,4]oxazine;
6-methyl-4-[3-(6-methyl-3-pyridyl)-7,8-dihydro-5H-1,6-naphthyridin-6-yl]thieno[2,3-d]pyrimidine;
7-methyl-4-[3-(6-methyl-3-pyridyl)-7,8-dihydro-5H-1,6-naphthyridin-6-yl]thieno[3,2-d]pyrimidine;
6-(2-cyclopropyl-6,7-dihydro-5H-cyclopenta[d]pyrimidin-4-yl)-3-(trifluoromethyl)-7,8-dihydro-5H-1,6-naphthyridine;
6-(2-methyl-6,7-dihydro-5H-cyclopenta[d]pyrimidin-4-yl)-3-(trifluoromethyl)-7,8-dihydro-5H-1,6-naphthyridine;
1-[6-(5-methylfuro[2,3-d]pyrimidin-4-yl)-7,8-dihydro-5H-1,6-naphthyridin-3-yl]-2,3-dihydropyrido[2,3-b][1,4]oxazine;
5-methyl-4-[3-(6-methyl-3-pyridyl)-7,8-dihydro-5H-1,6-naphthyridin-6-yl]furo[2,3-d]pyrimidine;
5-methyl-4-[3-(2-methylthiazol-5-yl)-7,8-dihydro-5H-1,6-naphthyridin-6-yl]furo[2,3-d]pyrimidine;
1-[6-(5-methylthieno[2,3-d]pyrimidin-4-yl)-7,8-dihydro-5H-1,6-naphthyridin-3-yl]-2,3-dihydropyrido[2,3-b][1,4]oxazine;
5-methyl-4-[3-(6-methyl-3-pyridyl)-7,8-dihydro-5H-1,6-naphthyridin-6-yl]thieno[2,3-d]pyrimidine;
5-methyl-4-[3-(2-methylthiazol-5-yl)-7,8-dihydro-5H-1,6-naphthyridin-6-yl]thieno[2,3-d]pyrimidine;
1-[6-(3-methylisoxazolo[5,4-d]pyrimidin-4-yl)-7,8-dihydro-5H-1,6-naphthyridin-3-yl]-2,3-dihydropyrido[2,3-b][1,4]oxazine;
3-methyl-4-[3-(6-methyl-3-pyridyl)-7,8-dihydro-5H-1,6-naphthyridin-6-yl]isoxazolo[5,4-d]pyrimidine;
3-methyl-4-[3-(2-methylthiazol-5-yl)-7,8-dihydro-5H-1,6-naphthyridin-6-yl]isoxazolo[5,4-d]pyrimidine;
4-[3-(2-fluoro-4-methyl-phenoxy)-7,8-dihydro-5H-1,6-naphthyridin-6-yl]-5-methyl-thieno[2,3-d]pyrimidine;
4-[3-(6,7-dihydro-5H-pyrazolo[1,5-a]pyrimidin-4-yl)-7,8-dihydro-5H-1,6-naphthyridin-6-yl]-6-methyl-thieno[2,3-d]pyrimidine;
4-[3-(1-cyclopropylpyrazol-4-yl)-7,8-dihydro-5H-1,6-naphthyridin-6-yl]-6-methyl-thieno[2,3-d]pyrimidine;
6-methyl-4-[3-(2-methylthiazol-5-yl)-7,8-dihydro-5H-1,6-naphthyridin-6-yl]thieno[2,3-d]pyrimidine;
4-[3-(6,7-dihydro-5H-pyrazolo[1,5-a]pyrimidin-4-yl)-7,8-dihydro-5H-1,6-naphthyridin-6-yl]-7-methyl-thieno[3,2-d]pyrimidine;
4-[3-(1-cyclopropylpyrazol-4-yl)-7,8-dihydro-5H-1,6-naphthyridin-6-yl]-7-methyl-thieno[3,2-d]pyrimidine;
4-[3-(1,3-dimethylpyrazol-4-yl)-7,8-dihydro-5H-1,6-naphthyridin-6-yl]-7-methyl-thieno[3,2-d]pyrimidine;
1-[6-(7-methylthieno[3,2-d]pyrimidin-4-yl)-7,8-dihydro-5H-1,6-naphthyridin-3-yl]-2,3-dihydropyrido[2,3-b][1,4]oxazine;
7-methyl-4-[3-(2-methylthiazol-5-yl)-7,8-dihydro-5H-1,6-naphthyridin-6-yl]thieno[3,2-d]pyrimidine;
4-[3-(6,7-dihydro-5H-pyrazolo[1,5-a]pyrimidin-4-yl)-7,8-dihydro-5H-1,6-naphthyridin-6-yl]-5-methyl-thieno[2,3-d]pyrimidine;
4-[3-(1-cyclopropylpyrazol-4-yl)-7,8-dihydro-5H-1,6-naphthyridin-6-yl]-5-methyl-thieno[2,3-d]pyrimidine;
4-[3-(2-fluoro-5-methyl-phenoxy)-7,8-dihydro-5H-1,6-naphthyridin-6-yl]thieno[3,2-d]pyrimidine;
4-[3-[(2-fluorophenyl)methyl]-7,8-dihydro-5H-1,6-naphthyridin-6-yl]-5-methyl-furo[2,3-d]pyrimidine;
4-[3-(1,3-dimethylpyrazol-4-yl)-7,8-dihydro-5H-1,6-naphthyridin-6-yl]-5-methyl-thieno[2,3-d]pyrimidine;
5-bromo-4-[3-(trifluoromethyl)-7,8-dihydro-5H-1,6-naphthyridin-6-yl]thieno[2,3-d]pyrimidine;
6-bromo-4-[3-(trifluoromethyl)-7,8-dihydro-5H-1,6-naphthyridin-6-yl]thieno[2,3-d]pyrimidine;
4-[3-(6,7-dihydro-5H-pyrazolo[1,5-a]pyrimidin-4-yl)-7,8-dihydro-5H-1,6-naphthyridin-6-yl]-6-isopropyl-thieno[2,3-d]pyrimidine;
4-[3-[(3-fluorophenyl)methyl]-7,8-dihydro-5H-1,6-naphthyridin-6-yl]-5-methyl-furo[2,3-d]pyrimidine;
4-[3-(2-fluorophenoxy)-7,8-dihydro-5H-1,6-naphthyridin-6-yl]-5-methyl-thieno[2,3-d]pyrimidine;
4-[3-(2-fluorophenoxy)-7,8-dihydro-5H-1,6-naphthyridin-6-yl]thieno[2,3-d]pyrimidine;
4-[3-(2-methylthiazol-5-yl)-7,8-dihydro-5H-1,6-naphthyridin-6-yl]thieno[2,3-d]pyrimidine;
N-(2-methylpyrazol-3-yl)-6-thieno[2,3-d]pyrimidin-4-yl-7,8-dihydro-5H-1,6-naphthyridin-3-amine;
4-[6-(6-isopropylthieno[2,3-d]pyrimidin-4-yl)-7,8-dihydro-5H-1,6-naphthyridin-3-yl]-3,5-dimethyl-isoxazole;
5-deuterio-4-[3-(trifluoromethyl)-7,8-dihydro-5H-1,6-naphthyridin-6-yl]thieno[2,3-d]pyrimidine;
4-[3-(2-fluorophenoxy)-7,8-dihydro-5H-1,6-naphthyridin-6-yl]-6-methyl-thieno[2,3-d]pyrimidine;
5-cyclopropyl-4-[3-(trifluoromethyl)-7,8-dihydro-5H-1,6-naphthyridin-6-yl]thieno[2,3-d]pyrimidine;
6-(5-methylfuro[2,3-d]pyrimidin-4-yl)-N-(2-methylpyrazol-3-yl)-7,8-dihydro-5H-1,6-naphthyridin-3-amine;
N-(2-fluoro-3-pyridyl)-6-(5-methylfuro[2,3-d]pyrimidin-4-yl)-7,8-dihydro-5H-1,6-naphthyridin-3-amine;
5-methyl-4-[3-(2-methylpyrazol-3-yl)-7,8-dihydro-5H-1,6-naphthyridin-6-yl]furo[2,3-d]pyrimidine;
2-chloro-4-[3-(trifluoromethyl)-7,8-dihydro-5H-1,6-naphthyridin-6-yl]-5,7-dihydrofuro[3,4-d]pyrimidine;
1-[6-(2-chloro-5,7-dihydrofuro[3,4-d]pyrimidin-4-yl)-7,8-dihydro-5H-1,6-naphthyridin-3-yl]-2,3-dihydropyrido[2,3-b][1,4]oxazine;
2-chloro-4-(3-(6,7-dihydropyrazolo[1,5-a]pyrimidin-4(5H)-yl)-7,8-dihydro-1,6-naphthyridin-6(5H)-yl)-5,7-dihydrofuro[3,4-d]pyrimidine;
(R)-2-chloro-4-(3-(2-methylmorpholino)-7,8-dihydro-1,6-naphthyridin-6(5H)-yl)-5,7-dihydrofuro[3,4-d]pyrimidine;
2-chloro-4-(3-cyclopropyl-7,8-dihydro-1,6-naphthyridin-6(5H)-yl)-5,7-dihydrofuro[3,4-d]pyrimidine;
6-(2-chloro-5,7-dihydrofuro[3,4-d]pyrimidin-4-yl)-N-(1-methyl-1H-pyrazol-5-yl)-5,6,7,8-tetrahydro-1,6-naphthyridin-3-amine;
6-(2-chloro-5,7-dihydrofuro[3,4-d]pyrimidin-4-yl)-N-(2-fluoropyridin-3-yl)-5,6,7,8-tetrahydro-1,6-naphthyridin-3-amine; and
2-chloro-4-(3-(2-fluorophenoxy)-7,8-dihydro-1,6-naphthyridin-6(5H)-yl)-5,7-dihydrofuro[3,4-d]pyrimidine,
   or a pharmaceutically acceptable salt thereof.

E47. The compound of any one of E1 to E46, or a pharmaceutically acceptable salt thereof, wherein the compound is isotopically labeled.

E48. A pharmaceutical composition comprising a compound of any one of E1 to E47, or a pharmaceutically acceptable salt thereof, and a pharmaceutically acceptable carrier.

E49. A method for treating a neurological and/or psychiatric disorder associated with muscarinic acetylcholine receptor dysfunction in a patient in need thereof, comprising a step of administering to the patient a therapeutically effective amount of a compound, or pharmaceutically acceptable salt thereof, or pharmaceutical composition according to any one of E1 to E48.

E50. The method of E49, wherein the disorder is selected from Alzheimer's disease, schizophrenia, a sleep disorder, borderline personality disorder, Tourette's syndrome, bipolar disorder, tardive dyskinesia, Huntington's disease, a pain disorder, and a cognitive disorder.

E51. The method of E50, wherein the disorder is Alzheimer's disease.

E52. The method of E50, wherein the disorder is schizophrenia.

E53. The method of E49, wherein the disorder is selected from psychosis, schizophrenia, conduct disorder, disruptive behavior disorder, bipolar disorder, psychotic episodes of anxiety, anxiety associated with psychosis, psychotic mood disorders such as severe major depressive disorder; mood disorders associated with psychotic disorders, acute mania, depression associated with bipolar disorder, mood disorders associated with schizophrenia, behavioral manifestations of mental retardation, autistic disorder, movement disorders, Tourette's syndrome, akinetic-rigid syndrome, movement disorders associated with Parkinson's disease, tardive dyskinesia, drug induced and neurodegeneration based dyskinesias, attention deficit hyperactivity disorder, cognitive disorders, dementias, and memory disorders.

E54. A compound, or a pharmaceutically acceptable salt thereof according to E1 to E47 for use in therapy.

E55. A compound, or a pharmaceutically acceptable salt thereof, or a pharmaceutical composition according to any one of claims 1 to 48 for use in the treatment of a disorder selected from Alzheimer's disease, schizophrenia, borderline personality disorder, Tourette's syndrome, bipolar disorder, tardive dyskinesia, Huntington's disease, a sleep disorder, a pain disorder, and a cognitive disorder.

E56. A compound, or a pharmaceutically acceptable salt thereof, or a pharmaceutical composition according to any one of E1 to E48 for use in the treatment of a disorder selected from psychosis, schizophrenia, conduct disorder, disruptive behavior disorder, bipolar disorder, psychotic episodes of anxiety, anxiety associated with psychosis, psychotic mood disorders such as severe major depressive disorder; mood disorders associated with psychotic disorders, acute mania, depression associated with bipolar disorder, mood disorders associated with schizophrenia, behavioral manifestations of mental retardation, autistic disorder, movement disorders, Tourette's syndrome, akinetic-rigid syndrome, movement disorders associated with Parkinson's disease, tardive dyskinesia, drug induced and neurodegeneration based dyskinesias, attention deficit hyperactivity disorder, cognitive disorders, dementias, and memory disorders.

E57. Use of a compound, or a pharmaceutically acceptable salt thereof, or a pharmaceutical composition according to any one of E1 to E48 in the manufacture of a medicament for the treatment of a disorder selected from Alzheimer's disease, schizophrenia, borderline personality disorder, Tourette's syndrome, bipolar disorder, tardive dyskinesia, Huntington's disease a sleep disorder, a pain disorder, and a cognitive disorder.

E58. Use of a compound, or a pharmaceutically acceptable salt thereof, or a pharmaceutical composition according to any one of E1 to E48 in the manufacture of a medicament for the treatment of a disorder selected from psychosis, schizophrenia, conduct disorder, disruptive behavior disorder, bipolar disorder, psychotic episodes of anxiety, anxiety associated with psychosis, psychotic mood disorders such as severe major depressive disorder; mood disorders associated with psychotic disorders, acute mania, depression associated with bipolar disorder, mood disorders associated with schizophrenia, behavioral manifestations of mental retardation, autistic disorder, movement disorders, Tourette's syndrome, akinetic-rigid syndrome, movement disorders associated with Parkinson's disease, tardive dyskinesia, drug induced and neurodegeneration based dyskinesias, attention deficit hyperactivity disorder, cognitive disorders, dementias, and memory disorders.

E59. A kit comprising a compound, or a pharmaceutically acceptable salt thereof, or a pharmaceutical composition of any one of E1 to E48, and one or more of: (a) at least one agent known to increase mAChR M₄ activity; (b) at least one agent known to decrease mAChR M₄ activity; (c) at least one agent known to treat a disorder associated with cholinergic activity; (d) instructions for treating a disorder associated with cholinergic activity; (e) instructions for treating a disorder associated with mAChR M₄ receptor activity; and (f) instructions for administering the compound in connection with cognitive or behavioral therapy.

It should be understood that the compounds of the invention may possess tautomeric forms, stereoisomers, geometric isomers, solvates, hydrates as well as polymorphs, and that these also constitute embodiments of the invention.

In the compounds of formula (I), and any subformulas, any “hydrogen” or “H,” whether explicitly recited or implicit in the structure, encompasses hydrogen isotopes ¹H (protium) and ²H (deuterium).

The present invention also includes an isotopically-labeled compound, which have the same chemical formula to those recited in formula (I), formula (Ia), formula (Ib), formula (Ic), and formula (Id) but for the fact that one or more atoms are replaced by an atom having an atomic mass or mass number different from the atomic mass or mass number usually found in nature. Examples of isotopes suitable for inclusion in the compounds of the invention are hydrogen, carbon, nitrogen, oxygen, phosphorus, sulfur, fluorine, and chlorine, such as, but not limited to ²H, ¹³C, ¹⁴C, ¹⁵N, ¹⁸O, ¹⁷O, ³¹P, ³²P, ³⁵S, ¹⁸F, and ³⁶Cl, respectively. Substitution with heavier isotopes such as deuterium, i.e. ²H, may afford certain therapeutic advantages resulting from greater metabolic stability, for example increased in vivo half-life or reduced dosage requirements and, hence, may be preferred in some circumstances. The compound may incorporate positron-emitting isotopes for medical imaging and positron-emitting tomography (PET) studies for determining the distribution of receptors. Suitable positron emitting isotopes that can be incorporated in compounds of formula (I) are ¹¹C, ¹³N, ¹⁵O, and ¹⁸F. Isotopically-labeled compounds of formula (I) can generally be prepared by conventional techniques known to those skilled in the art or by processes analogous to those described in the accompanying Examples using appropriate isotopically-labeled reagent in place of nonisotopically-labeled reagent.

In some embodiments, R³, R⁴, R⁵ are deuterium.

In some embodiments, R² is deuterium.

In some embodiments, R⁵ is deuterium.

a. Pharmaceutically Acceptable Salts

The compounds of the invention may exist as pharmaceutically acceptable salts. The term “pharmaceutically acceptable salt” refers to salts or zwitterions of the compounds, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides and iodides such as methyl, ethyl, propyl, isopropyl, butyl, lauryl, myristyl, stearyl and the like.

Basic addition salts may be prepared during the final isolation and purification of the compounds of the invention by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

### 3. PHARMACEUTICAL COMPOSITIONS AND FORMULATIONS

The compounds of the invention may be incorporated into pharmaceutical compositions suitable for administration to a subject (such as a patient, which may be a human or non-human). The compounds of the invention may also be provided as formulations, such as spray-dried dispersion formulations.

The pharmaceutical compositions and formulations may include a “therapeutically effective amount” or a “prophylactically effective amount” of the agent. A “therapeutically effective amount” refers to an amount effective, at single or multiple dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the invention (e.g., a compound of formula (I)) are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

For example, a therapeutically effective amount of a compound of formula (I), may be about 0.01 to about 500 mg per kg, about 0.1 to about 250 mg/kg, about 0.5 to about 100 mg/kg, about 0.01 to about 250 mg/kg, about 0.05 to about 100 mg/kg, about 0.1 to about 50 mg/kg, about 0.05 to about 0.5 mg/kg, about 0.5 to about 5 mg/kg, about 5 to about 50 mg/kg, about 1 mg/kg to about 1000 mg/kg, about 5 mg/kg to about 950 mg/kg, about 10 mg/kg to about 900 mg/kg, about 15 mg/kg to about 850 mg/kg, about 20 mg/kg to about 800 mg/kg, about 25 mg/kg to about 750 mg/kg, about 30 mg/kg to about 700 mg/kg, about 35 mg/kg to about 650 mg/kg, about 40 mg/kg to about 600 mg/kg, about 45 mg/kg to about 550 mg/kg, about 50 mg/kg to about 500 mg/kg, about 55 mg/kg to about 450 mg/kg, about 60 mg/kg to about 400 mg/kg, about 65 mg/kg to about 350 mg/kg, about 70 mg/kg to about 300 mg/kg, about 75 mg/kg to about 250 mg/kg, about 80 mg/kg to about 200 mg/kg, about 85 mg/kg to about 150 mg/kg, and about 90 mg/kg to about 100 mg/kg.

The pharmaceutical compositions and formulations may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the compounds and their pharmaceutically acceptable salts may be formulated for administration by, for example, solid dosing, eye drop, in a topical oil-based formulation, injection, inhalation (either through the mouth or the nose), implants, or oral, buccal, parenteral, or rectal administration. Techniques and formulations may generally be found in “Remington's Pharmaceutical Sciences,” (Meade Publishing Co., Easton, Pa.). Therapeutic compositions must typically be sealed and stable under the conditions of manufacture and storage.

The route by which the compounds of the invention are administered and the form of the composition will dictate the type of carrier to be used. The composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90 weight % of the total composition weight.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10 weight % of the total composition weight.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50% of the total composition weight.

Suitable disintegrants include agar, alginic acid and the sodium salt thereof, effervescent mixtures, croscarmellose, crospovidone, sodium carboxymethyl starch, sodium starch glycolate, clays, and ion exchange resins. The amount of disintegrant(s) in a systemic or topical composition is typically about 0.1 to about 10 weight % of the total composition weight.

Suitable colorants include a colorant such as an FD&C dye. When used, the amount of colorant in a systemic or topical composition is typically about 0.005 to about 0.1 weight % of the total composition weight.

Suitable flavors include menthol, peppermint, and fruit flavors. The amount of flavor(s), when used, in a systemic or topical composition is typically about 0.1 to about 1.0 weight % of the total composition weight.

Suitable sweeteners include aspartame and saccharin. The amount of sweetener(s) in a systemic or topical composition is typically about 0.001 to about 1 weight % of the total composition weight.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5 weight % of the total composition weight.

Suitable preservatives include benzalkonium chloride, methyl paraben and sodium benzoate. The amount of preservative(s) in a systemic or topical composition is typically about 0.01 to about 5%.

Suitable glidants include silicon dioxide. The amount of glidant(s) in a systemic or topical composition is typically about 1 to about 5 weight % of the total composition weight.

Suitable solvents include water, isotonic saline, ethyl oleate, glycerine, hydroxylated castor oils, alcohols such as ethanol, and phosphate buffer solutions. The amount of solvent(s) in a systemic or topical composition is typically from about 0 to about 100 weight % of the total composition weight.

Suitable suspending agents include AVICEL RC-591 (from FMC Corporation of Philadelphia, PA) and sodium alginate. The amount of suspending agent(s) in a systemic or topical composition is typically about 1 to about 8 weight % of the total composition weight.

Suitable surfactants include lecithin, Polysorbate 80, and sodium lauryl sulfate, and the TWEENS from Atlas Powder Company of Wilmington, Delaware. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1 to about 5 weight % of the total composition weight.

Although the amounts of components in the systemic compositions may vary depending on the type of systemic composition prepared, in general, systemic compositions include 0.01 to 50 weight % of the total composition weight of an active compound (i.e., a compound of formula (I) or a pharmaceutically acceptable salt thereof) and 50 to 99.99 weight % of the total composition weight of one or more carriers. Compositions for parenteral administration typically include 0.1 to 10 weight % of the total composition weight of active compounds and 90 to 99.9 weight % of the total composition weight of a carrier including a diluent and a solvent.

Compositions for oral administration can have various dosage forms. For example, solid forms include tablets, capsules, granules, and bulk powders. These oral dosage forms include a safe and effective amount, usually at least about 5 weight % of the total composition weight, and more particularly from about 25 to about 50 weight % of the total composition weight of active compounds. The oral dosage compositions typically include about 50 to about 95 weight % of the total composition weight of carriers, and more particularly, from about 50 to about 75 weight % of the total composition weight.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose and cellulose. Specific binders include starch, gelatin, and sucrose. Specific disintegrants include alginic acid and croscarmellose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound (i.e., a compound of formula (I) or a pharmaceutically acceptable salt thereof), and a carrier including one or more diluents disclosed above in a capsule comprising gelatin. Granules typically comprise a compound of the invention, and preferably a glidant such as silicon dioxide to improve flow characteristics. Implants can be of the biodegradable or the non-biodegradable type.

The selection of ingredients in the carrier for oral compositions depends on secondary considerations like taste, cost, and shelf stability, which are not critical for the purposes of this invention.

Solid compositions may be coated by conventional methods, typically with pH or time-dependent coatings, such that a compound of the invention is released in the gastrointestinal tract in the vicinity of the desired application, or at various points and times to extend the desired action. The coatings typically include one or more components selected from cellulose acetate phthalate, polyvinyl acetate phthalate, hydroxypropyl methyl cellulose phthalate, ethyl cellulose, EUDRAGIT® coatings (available from Evonik Industries of Essen, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a compound of the invention and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The compounds of the invention can be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions include: a compound of the invention (e.g., a compound of formula (I)), and a carrier. The carrier of the topical composition preferably aids penetration of the compounds into the skin. The carrier may further include one or more optional components.

The amount of the carrier employed in conjunction with a compound of the invention is sufficient to provide a practical quantity of composition for administration per unit dose of the compound. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5 to about 95 weight % of the total composition weight.

Suitable propellants include propane, butane, isobutane, dimethyl ether, carbon dioxide, nitrous oxide, and combinations thereof. The amount of propellant(s) in a topical composition is typically about 0 to about 95 weight % of the total composition weight.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0 to about 95 weight % of the total composition weight.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0 to 95 weight % of the total composition weight.

The amount of thickener(s) in a topical composition is typically about 0 to about 95 weight % of the total composition weight.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0 to 95 weight % of the total composition weight.

The amount of fragrance in a topical composition is typically about 0 to about 0.5 weight %, particularly, about 0.001 to about 0.1 weight % of the total composition weight.

Suitable pH adjusting additives include HCl or tH in amounts sufficient to adjust the pH of a topical pharmaceutical composition.

The pharmaceutical composition or formulation may exhibit positive allosteric modulation of mAChR M₄ with an EC₅₀ of less than about 10 μM, less than about 5 μM, less than about 1 μM, less than about 500 nM, or less than about 100 nM. The pharmaceutical composition or formulation may exhibit positive allosteric modulation of mAChR M₄ with an EC₅₀ of between about 10 μM and about 1 nM, about 1 μM and about 1 nM, about 100 nM and about 1 nM, or between about 10 nM and about 1 nM.

a. Spray-Dried Dispersion Formulations

The compounds of the invention may be formulated as a spray-dried dispersion (SDD). An SDD is a single-phase, amorphous molecular dispersion of a drug in a polymer matrix. It is a solid solution with the compound molecularly “dissolved” in a solid matrix. SDDs are obtained by dissolving drug and a polymer in an organic solvent and then spray-drying the solution. The use of spray drying for pharmaceutical applications can result in amorphous dispersions with increased solubility of Biopharmaceutics Classification System (BCS) class II (high permeability, low solubility) and class IV (low permeability, low solubility) drugs. Formulation and process conditions are selected so that the solvent quickly evaporates from the droplets, thus allowing insufficient time for phase separation or crystallization. SDDs have demonstrated long-term stability and manufacturability. For example, shelf lives of more than 2 years have been demonstrated with SDDs. Advantages of SDDs include, but are not limited to, enhanced oral bioavailability of poorly water-soluble compounds, delivery using traditional solid dosage forms (e.g., tablets and capsules), a reproducible, controllable and scalable manufacturing process and broad applicability to structurally diverse insoluble compounds with a wide range of physical properties.

Thus, in one embodiment, the invention may provide a spray-dried dispersion formulation comprising a compound of formula (I) or a pharmaceutically acceptable salt thereof.

### 4. METHODS OF USE

The compounds of the invention, pharmaceutical compositions and formulations may be used in methods for treatment of disorders, such as neurological and/or psychiatric disorders, associated with muscarinic acetylcholine receptor dysfunction. The compounds of the invention and pharmaceutical compositions may also be used in methods for the potentiation of muscarinic acetylcholine receptor activity in a mammal, and in methods for enhancing cognition in a mammal. The methods further include cotherapeutic methods for improving treatment outcomes in the context of cognitive or behavioral therapy. In the methods of use described herein, additional therapeutic agent(s) may be administered simultaneously or sequentially with the compounds of the invention and compositions.

a. Treating Disorders

The compounds of the invention, pharmaceutical compositions and formulations may be used in methods for treatment of disorders, such as neurological and/or psychiatric disorders, associated with muscarinic acetylcholine receptor dysfunction. The methods of treatment may comprise administering to a subject in need of such treatment a therapeutically effective amount of the compound of formula (I), a pharmaceutically acceptable salt thereof, or a pharmaceutical composition comprising a therapeutically effective amount of a compound of formula (I), or a pharmaceutically acceptable salt thereof.

In some embodiments, the invention provides a method for enhancing cognition in a mammal comprising the step of administering to the mammal a therapeutically effective amount of the compound of formula (I), a pharmaceutically acceptable salt thereof, or a pharmaceutical composition comprising a therapeutically effective amount of a compound of formula (I), or a pharmaceutically acceptable salt thereof.

The compounds of the invention and compositions disclosed herein may be useful for treating, preventing, ameliorating, controlling or reducing the risk of a variety of disorders associated with selective mAChR M₄ receptor activation. For example, a treatment can include selective mAChR M₄ receptor activation to an extent effective to affect cholinergic activity. A disorder can be associated with cholinergic activity, for example cholinergic hypofunction. Thus, provided is a method of treating or preventing a disorder in a subject comprising the step of administering to the subject at least one compound of the invention or at least one disclosed pharmaceutical composition, in an amount effective to treat the disorder in the subject.

Also provided is a method for the treatment of one or more disorders associated with mAChR M₄ receptor activity in a subject comprising the step of administering to the subject a therapeutically effective amount of the compound of formula (I), a pharmaceutically acceptable salt thereof, or a pharmaceutical composition comprising a therapeutically effective amount of a compound of formula (I), or a pharmaceutically acceptable salt thereof.

In some embodiments, the invention provides a method for the treatment of a disorder associated with muscarinic acetylcholine receptor dysfunction in a mammal, comprising the step of administering to the mammal an effective amount of at least one compound of the invention or a pharmaceutically acceptable salt thereof, or a pharmaceutical composition comprising at least one compound of the invention or pharmaceutically acceptable salt thereof.

In some embodiments, the compounds of the invention and compositions have utility in treating a variety of neurological, psychiatric and cognitive disorders associated with the mAChR M₄ receptor, including one or more of the following conditions or diseases: schizophrenia, psychotic disorder NOS, brief psychotic disorder, schizophreniform disorder, schizoaffective disorder, delusional disorder, shared psychotic disorder, catastrophic schizophrenia, postpartum psychosis, psychotic depression, psychotic break, tardive psychosis, myxedematous psychosis, occupational psychosis, menstrual psychosis, secondary psychotic disorder, bipolar I disorder with psychotic features, and substance-induced psychotic disorder. In some embodiments, the psychotic disorder is a psychosis associated with an illness selected from major depressive disorder, affective disorder, borderline personality disorder, bipolar disorder, electrolyte disorder, Alzheimer's disease, neurological disorder, hypoglycemia, AIDS, lupus, and post-traumatic stress disorder.

In some embodiments, the disorder is schizophrenia.

In some embodiments, the disorder is psychotic depression.

In some embodiments, the disorder is agitation and psychosis in Alzheimer's disease.

In some embodiment, the disorder is a bipolar disorder.

In some embodiments, the disorder is a neurological disorder is selected from brain tumor, dementia with Lewy bodies, multiple sclerosis, sarcoidosis, Lyme disease, syphilis, Alzheimer's disease, Parkinson's disease, and anti-NMDA receptor encephalitis.

In some embodiments, the disorder is a psychotic disorder is selected from schizophrenia, brief psychotic disorder, schizophreniform disorder, schizoaffective disorder, delusional disorder, and shared psychotic disorder. In some embodiments, the schizophrenia is selected from catastrophic schizophrenia, catatonic schizophrenia, paranoid schizophrenia, residual schizophrenia, disorganized schizophrenia, and undifferentiated schizophrenia. In some embodiments, the disorder is selected from schizoid personality disorder, schizotypal personality disorder, and paranoid personality disorder. In some embodiments, the psychotic disorder is due to a general medical condition and is substance-induced or drug-induced (phencyclidine, ketamine and other dissociative anesthetics, amphetamine and other psychostimulants, and cocaine).

In some embodiments, the present invention provides a method for treating a development disorder, comprising administering to a patient in need thereof an effective amount of a compound or a composition of the present invention. In some embodiments, the development disorder is fragile X syndrome.

In some embodiments, the present invention provides a method for treating a cognitive disorder, comprising administering to a patient in need thereof an effective amount of a compound or a composition of the present invention. In some embodiments, cognitive disorders include dementia (associated with Alzheimer's disease, ischemia, multi-infarct dementia, trauma, vascular problems or stroke, HIV disease, Parkinson's disease, Huntington's disease, Pick's disease, Creutzfeldt-Jacob disease, perinatal hypoxia, other general medical conditions or substance abuse), delirium, amnestic disorder, substance-induced persisting delirium, dementia due to HIV disease, dementia due to Huntington's disease, dementia due to Parkinson's disease, Parkinsonian-ALS demential complex, dementia of the Alzheimer's type, age-related cognitive decline, and mild cognitive impairment.

In some embodiments, the disorder is Huntington's disease.

The text revision of the fourth edition of the Diagnostic and Statistical Manual of Mental Disorders (DSM-IV-TR) (2000, American Psychiatric Association, Washington DC) provides a diagnostic tool that includes cognitive disorders including dementia, delirium, amnestic disorders and age-related cognitive decline. The fifth edition of the Diagnostic and Statistical Manual of Mental Disorders (DSM-5) (2013, American Psychiatric Association, Washington DC) provides a diagnostic tool for neurocognitive disorders (NCDs) that include delirium, followed by the syndromes of major NCD, mild NCD, and their etiological subtypes. The major or mild NCD subtypes include NCD due to Alzheimer's disease, vascular NCD, NCD with Lewy bodies, NCD due to Parkinson's disease, frontotemporal NCD, NCD due to traumatic brain injury, NCD due to HIV infection, substance/medication-induced NCD, NCD due to Huntington's disease, NCD due to prion disease, NCD due to another medical condition, NCD due to multiple etiologies, and unspecified NCD. The NCD category in DSM-5 encompasses the group of disorders in which the primary clinical deficit is in cognitive function, and that are acquired rather than developmental. As used herein, the term “cognitive disorders” includes treatment of those cognitive disorders and neurocognitive disorders as described in DSM-IV-TR or DSM-5. The skilled artisan will recognize that there are alternative nomenclatures, nosologies and classification systems for mental disorders, and that these systems evolve with medical and scientific progress. Thus the term “cognitive disorders” is intended to include like disorders that are described in other diagnostic sources.

In some embodiments, the present invention provides a method for treating schizophrenia or psychosis, comprising administering to a patient in need thereof an effective amount of a compound or composition of the present invention. Particular schizophrenia or psychosis pathologies are paranoid, disorganized, catatonic or undifferentiated schizophrenia and substance-induced psychotic disorder. DSM-IV-TR provides a diagnostic tool that includes paranoid, disorganized, catatonic, undifferentiated or residual schizophrenia, and substance-induced psychotic disorder. DSM-5 eliminated the subtypes of schizophrenia, and instead includes a dimensional approach to rating severity for the core symptoms of schizophrenia, to capture the heterogeneity in symptom type and severity expressed across individuals with psychotic disorders. As used herein, the term “schizophrenia or psychosis” includes treatment of those mental disorders as described in DSM-IV-TR or DSM-5. The skilled artisan will recognize that there are alternative nomenclatures, nosologies and classification systems for mental disorders, and that these systems evolve with medical and scientific progress. Thus the term “schizophrenia or psychosis” is intended to include like disorders that are described in other diagnostic sources.

In some embodiments, the present invention provides a method for treating sleep disorder, comprising administering to a patient in need thereof an effective amount of a compound or a pharmaceutical composition of the present invention.

In some embodiments, the sleep disorder is associated with sleep disturbance in a patient having schizophrenia.

In some embodiments, the present invention provides a method for treating patients at risk of developing schizophrenia, comprising administering to a patient in need thereof an effective amount of a compound or pharmaceutical composition of the present invention.

In some embodiments, the present invention provides a method for treating pain, comprising administering to a patient in need thereof an effective amount of a compound or composition of the present invention. Particular pain embodiments are bone and joint pain (osteoarthritis), repetitive motion pain, dental pain, cancer pain, myofascial pain (muscular injury, fibromyalgia), perioperative pain (general surgery, gynecological), chronic pain and neuropathic pain.

The compounds and compositions may be further useful in a method for the prevention, treatment, control, amelioration, or reduction of risk of the diseases, disorders and conditions noted herein. The compounds and compositions may be further useful in a method for the prevention, treatment, control, amelioration, or reduction of risk of the aforementioned diseases, disorders and conditions, in combination with other agents.

In the treatment of conditions which require activation of mAChR M₄, an appropriate dosage level may be about 0.01 to 500 mg per kg patient body weight per day, which can be administered in single or multiple doses. The dosage level may be about 0.1 to about 250 mg/kg per day, or about 0.5 to about 100 mg/kg per day. A suitable dosage level can be about 0.01 to 250 mg/kg per day, about 0.05 to 100 mg/kg per day, or about 0.1 to 50 mg/kg per day. Within this range the dosage can be 0.05 to 0.5, 0.5 to 5 or 5 to 50 mg/kg per day. For oral administration, the compositions may be provided in the form of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900, or 1000 milligrams of the active ingredient for the symptomatic adjustment of the dosage to the patient to be treated. The compounds can be administered on a regimen of 1 to 4 times per day, preferably once or twice per day. This dosage regimen can be adjusted to provide the optimal therapeutic response. It will be understood, however, that the specific dose level and frequency of dosage for any particular patient can be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Thus, in some embodiments, the invention relates to a method for activating mAChR M₄ receptor activity in at least one cell, comprising the step of contacting the at least one cell with at least one compound of the invention or pharmaceutical composition in an amount effective to activate mAChR M₄ in the at least one cell. In some embodiments, the cell is mammalian, for example, human. In some embodiments, the cell has been isolated from a subject prior to the contacting step. In some embodiments, contacting is via administration to a subject.

In some embodiments, the invention relates to a method for activating mAChR M₄ activity in a subject, comprising the step of administering to the subject at least one compound of the invention or at least one product of a disclosed method in a dosage and amount effective to activating mAChR M₄ activity in the subject. In some embodiments, the subject is mammalian, for example, human. In some embodiments, the mammal has been diagnosed with a need for mAChR M₄ agonism prior to the administering step. In some embodiments, the mammal has been diagnosed with a need for mAChR M₄ activation prior to the administering step. In some embodiments, the method further comprises the step of identifying a subject in need of mAChR M₄ agonism.

In some embodiments, the invention relates to a method for the treatment of a disorder associated with selective mAChR M₄ activation, for example, a disorder associated with cholinergic activity, in a mammal comprising the step of administering to the mammal at least one compound of the invention or at least one product of a disclosed method in a dosage and amount effective to treat the disorder in the mammal. In some embodiments, the mammal is a human. In some embodiments, the mammal has been diagnosed with a need for treatment for the disorder prior to the administering step. In some embodiments, the method further comprises the step of identifying a subject in need of treatment for the disorder.

In some embodiments, the disorder can be selected from psychosis, schizophrenia, conduct disorder, disruptive behavior disorder, bipolar disorder, psychotic episodes of anxiety, anxiety associated with psychosis, psychotic mood disorders such as severe major depressive disorder; mood disorders associated with psychotic disorders, acute mania, depression associated with bipolar disorder, mood disorders associated with schizophrenia, behavioral manifestations of mental retardation, autistic disorder, movement disorders, Tourette's syndrome, akinetic-rigid syndrome, movement disorders associated with Parkinson's disease, tardive dyskinesia, drug induced and neurodegeneration based dyskinesias, attention deficit hyperactivity disorder, obsessive-compulsive disorder, cognitive disorders, dementias, and memory disorders.

In some embodiments, the disorder is Alzheimer's disease.

In some embodiments, the disorder is Tourette's syndrome.

In some embodiments, the disorder is tardive dyskinesia.

In some embodiments, the disorder is obsessive-compulsive disorder.

In some embodiments, the disorder is bipolar disorder.

b. Potentiation of Muscarinic Acetylcholine Receptor Activity

In some embodiments, the invention relates to a method for potentiation of muscarinic acetylcholine receptor activity in a mammal comprising the step of administering to the mammal an effective amount of at least one compound of the invention or a pharmaceutically acceptable salt thereof, or a pharmaceutical composition comprising at least one compound of the invention or pharmaceutically acceptable salt thereof.

In some embodiments, potentiation of muscarinic acetylcholine receptor activity increases muscarinic acetylcholine receptor activity. In some embodiments, potentiation of muscarinic acetylcholine receptor activity is partial agonism of the muscarinic acetylcholine receptor. In some embodiments, potentiation of muscarinic acetylcholine receptor activity is positive allosteric modulation of the muscarinic acetylcholine receptor.

In some embodiments, the compound administered exhibits potentiation of mAChR M₄ with an EC₅₀ of less than about 10 μM, less than about 5 μM, less than about 1 μM, less than about 500 nM, or less than about 100 nM. In some embodiments, the compound administered exhibits potentiation of mAChR M₄ with an EC₅₀ of between about 10 μM and about 1 nM, about 1 μM and about 1 nM, about 100 nM and about 1 nM, or about 10 nM and about 1 nM.

In some embodiments, the mammal is a human. In some embodiments, the mammal has been diagnosed with a need for potentiation of muscarinic acetylcholine receptor activity prior to the administering step. In some embodiments, the method further comprises the step of identifying a mammal in need of potentiating muscarinic acetylcholine receptor activity. In some embodiments, the potentiation of muscarinic acetylcholine receptor activity treats a disorder associated with muscarinic acetylcholine receptor activity in the mammal. In some embodiments, the muscarinic acetylcholine receptor is mAChR M₄.

In some embodiments, potentiation of muscarinic acetylcholine receptor activity in a mammal is associated with the treatment of a neurological and/or psychiatric disorder associated with a muscarinic receptor dysfunction, such as a neurological or psychiatric disorder disclosed herein. In some embodiments, the muscarinic receptor is mAChR M₄.

In some embodiments, the invention provides to a method for potentiation of muscarinic acetylcholine receptor activity in a cell, comprising the step of contacting the cell with an effective amount of at least one compound of the invention or a pharmaceutically acceptable salt thereof. In some embodiments, the cell is mammalian (e.g., human). In some embodiments, the cell has been isolated from a mammal prior to the contacting step. In some embodiments, contacting is via administration to a mammal.

c. Enhancing Cognition

In some embodiments, the invention relates to a method for enhancing cognition in a mammal comprising the step of administering to the mammal an effective amount of at least one compound of the invention; or a pharmaceutically acceptable salt, hydrate, solvate, or polymorph thereof.

In some embodiments, the mammal is a human. In some embodiments, the mammal has been diagnosed with a need for cognition enhancement prior to the administering step. In some embodiments, the method further comprises the step of identifying a mammal in need of cognition enhancement. In some embodiments, the need for cognition enhancement is associated with a muscarinic receptor dysfunction. In some embodiments, the muscarinic receptor is mAChR M₄. In some embodiments, the need for cognition enhancement is associated with schizophrenia.

Cognitive deficits or cognitive impairment include a decline in cognitive functions or cognitive domains, e.g. working memory, attention and vigilance, verbal learning and memory, visual learning and memory, reasoning and problem solving e.g. executive function, speed of processing and/or social cognition. In particular, cognitive deficits or cognitive impairment may indicate deficits in attention, disorganized thinking, slow thinking, difficulty in understanding, poor concentration, impairment of problem solving, poor memory, difficulties in expressing thoughts and/or difficulties in integrating thoughts, feelings and behaviour, or difficulties in extinction of irrelevant thoughts. The terms “cognitive deficits” and “cognitive impairment” are intended to indicate the same and are used interchangeably.

The skilled person is familiar with various test for measuring the enhancement of cognition. Examples of test for measuring the enhancement of cognition are but not limited to the Novel Object Recognition and the Wisconsin Card Sorting Test.

d. Co-Therapeutic Methods

The present invention is further directed to administration of a selective mAChR M₄ activator for improving treatment outcomes in the context of cognitive or behavioral therapy. That is, in some embodiments, the invention relates to a cotherapeutic method comprising a step of administering to a mammal an effective amount and dosage of at least one compound of the invention, or a pharmaceutically acceptable salt thereof.

In some embodiments, administration improves treatment outcomes in the context of cognitive or behavioral therapy. Administration in connection with cognitive or behavioral therapy can be continuous or intermittent. Administration need not be simultaneous with therapy and can be before, during, and/or after therapy. For example, cognitive or behavioral therapy can be provided within 1, 2, 3, 4, 5, 6, 7 days before or after administration of the compound. As a further example, cognitive or behavioral therapy can be provided within 1, 2, 3, or 4 weeks before or after administration of the compound. As a still further example, cognitive or behavioral therapy can be provided before or after administration within a period of time of 1, 2, 3, 4, 5, 6, 7, 8, 9, or 10 half-lives of the administered compound.

It is understood that the disclosed cotherapeutic methods can be used in connection with the compounds of the invention, compositions, kits, and uses.

e. Combination Therapies

In the methods of use described herein, additional therapeutic agent(s) may be administered simultaneously or sequentially with the compounds and compositions of the invention. Sequential administration includes administration before or after the compounds of the invention and compositions. In some embodiments, the additional therapeutic agent or agents may be administered in the same composition as the compounds of the invention. In other embodiments, there may be an interval of time between administration of the additional therapeutic agent and the compounds of the invention. In some embodiments, administration of an additional therapeutic agent with a compound of the invention may allow lower doses of the other therapeutic agents and/or administration at less frequent intervals. When used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula (I). The above combinations include combinations of a compound of the present invention not only with one other active compound, but also with two or more other active compounds.

The compounds of the invention can be used as single agents or in combination with one or more other drugs in the treatment, prevention, control, amelioration or reduction of risk of the aforementioned diseases, disorders and conditions for which the compound or the other drugs have utility, where the combination of drugs together are safer or more effective than either drug alone. The other drug(s) can be administered by a route and in an amount commonly used therefor, contemporaneously or sequentially with a compound of the invention. When a compound of the invention is used contemporaneously with one or more other drugs, a pharmaceutical composition in unit dosage form containing such drugs and the compound of the invention may be used. However, the combination therapy can also be administered on overlapping schedules. It is also envisioned that the combination of one or more active ingredients and a compound of the invention can be more efficacious than either as a single agent. Thus, when used in combination with one or more other active ingredients, the compounds of the invention and the other active ingredients may be used in lower doses than when each is used singly.

The pharmaceutical compositions and methods of the present invention can further comprise other therapeutically active compounds as noted herein which are usually applied in the treatment of the above mentioned pathological conditions.

The above combinations include combinations of a compound of the invention not only with one other active compound, but also with two or more other active compounds. Likewise, compounds of the invention can be used in combination with other drugs that are used in the prevention, treatment, control, amelioration, or reduction of risk of the diseases or conditions for which compounds of the invention are useful. Such other drugs can be administered, by a route and in an amount commonly used therefor, contemporaneously or sequentially with a compound of the present invention. When a compound of the present invention is used contemporaneously with one or more other drugs, a pharmaceutical composition containing such other drugs in addition to a compound of the invention is preferred. Accordingly, the pharmaceutical compositions include those that also contain one or more other active ingredients, in addition to a compound of the present invention.

The weight ratio of a compound of the invention to the second active ingredient can be varied and will depend upon the effective dose of each ingredient. Generally, an effective dose of each will be used. Thus, for example, when a compound of the present invention is combined with another agent, the weight ratio of a compound of the invention to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations, a compound of the invention and other active agents can be administered separately or in conjunction. In addition, the administration of one element can be prior to, concurrent to, or subsequent to the administration of other agent(s).

Accordingly, the compounds of the invention can be used alone or in combination with other agents which are known to be beneficial in the subject indications or other drugs that affect receptors or enzymes that either increase the efficacy, safety, convenience, or reduce unwanted side effects or toxicity of the compounds of the invention. The subject compound and the other agent can be coadministered, either in concomitant therapy or in a fixed combination.

In some embodiments, the compound can be employed in combination with anti-Alzheimer's agents, beta-secretase inhibitors, cholinergic agents, gamma-secretase inhibitors, HMG-CoA reductase inhibitors, M₁ allosteric agonists, M₁ positive allosteric modulators, NSAIDs including ibuprofen, vitamin E, and anti-amyloid antibodies. In another embodiment, the subject compound can be employed in combination with sedatives, hypnotics, anxiolytics, antipsychotics (typical and atypical), antianxiety agents, cyclopyrrolones, imidazopyridines, pyrazolopyrimidines, minor tranquilizers, melatonin agonists and antagonists, melatonergic agents, benzodiazepines, barbiturates, 5HT-2 antagonists, and the like, such as: adinazolam, allobarbital, alonimid, alprazolam, amisulpride, amitriptyline, amobarbital, amoxapine, aripiprazole, bentazepam, benzoctamine, brotizolam, bupropion, busprione, butabarbital, butalbital, brexpiprazole, capuride, carbocloral, chloral betaine, chloral hydrate, clomipramine, clonazepam, cloperidone, clorazepate, chlordiazepoxide, clorethate, chlorpromazine, clozapine, cyprazepam, desipramine, dexclamol, diazepam, dichloralphenazone, divalproex, diphenhydramine, donepezil, memantine, galantamine, doxepin, estazolam, ethchlorvynol, etomidate, fenobam, flunitrazepam, zuflupentixol, flupentixol, fluphenazine, flurazepam, fluvoxamine, fluoxetine, fosazepam, glutethimide, halazepam, haloperidol, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, olanzapine, oxazepam, paraldehyde, paroxetine, pentobarbital, perlapine, perphenazine, phenelzine, phenobarbital, prazepam, promethazine, propofol, protriptyline, quazepam, quetiapine, reclazepam, risperidone, roletamide, secobarbital, sertraline, suproclone, temazepam, thioridazine, thiothixene, tracazolate, tranylcypromaine, trazodone, triazolam, trepipam, tricetamide, triclofos, trifluoperazine, trimetozine, trimipramine, uldazepam, zaleplon, ziprasidone, zolazepam, zolpidem, and salts thereof, and combinations thereof, and the like, or the subject compound can be administered in conjunction with the use of physical methods such as with light therapy or electrical stimulation.

In some embodiments, the compound can be employed in combination with levodopa (with or without a selective extracerebral decarboxylase inhibitor such as carbidopa or benserazide), anticholinergics such as biperiden (optionally as its hydrochloride or lactate salt) and trihexyphenidyl (benzhexol) hydrochloride, COMT inhibitors such as entacapone, MOA-B inhibitors such as azilect, antioxidants, A2a adenosine receptor antagonists such as istradefylline, cholinergic agonists, NMDA receptor antagonists such as ketamine, serotonin receptor antagonists and dopamine receptor agonists such as alentemol, bromocriptine, fenoldopam, lisuride, naxagolide, pergolide and pramipexole. It will be appreciated that the dopamine agonist can be in the form of a pharmaceutically acceptable salt, for example, alentemol hydrobromide, bromocriptine mesylate, fenoldopam mesylate, naxagolide hydrochloride and pergolide mesylate. Lisuride and pramipexol are commonly used in a non-salt form.

In some embodiments, the compound can be employed in combination with a compound from the phenothiazine, thioxanthene, heterocyclic dibenzazepine, butyrophenone, diphenylbutylpiperidine and indolone classes of neuroleptic agent. Suitable examples of phenothiazines include chlorpromazine, mesoridazine, thioridazine, acetophenazine, fluphenazine, perphenazine and trifluoperazine. Suitable examples of thioxanthenes include chlorprothixene and thiothixene. An example of a dibenzazepine is clozapine. An example of a butyrophenone is haloperidol. An example of a diphenylbutylpiperidine is pimozide. An example of an indolone is molindolone. Other neuroleptic agents include loxapine, sulpiride and risperidone. It will be appreciated that the neuroleptic agents when used in combination with the subject compound can be in the form of a pharmaceutically acceptable salt, for example, chlorpromazine hydrochloride, mesoridazine besylate, thioridazine hydrochloride, acetophenazine maleate, fluphenazine hydrochloride, flurphenazine enathate, fluphenazine decanoate, trifluoperazine hydrochloride, thiothixene hydrochloride, haloperidol decanoate, loxapine succinate and molindone hydrochloride. Perphenazine, chlorprothixene, clozapine, haloperidol, pimozide and risperidone are commonly used in a non-salt form. Thus, the subject compound can be employed in combination with acetophenazine, alentemol, amisulpride, benzhexol, bromocriptine, biperiden, chlorpromazine, chlorprothixene, clozapine, diazepam, fenoldopam, fluphenazine, haloperidol, levodopa, levodopa with benserazide, levodopa with carbidopa, lisuride, loxapine, mesoridazine, molindolone, naxagolide, olanzapine, pergolide, perphenazine, pimozide, pramipexole, quetiapine, risperidone, sulpiride, tetrabenazine, deutetrabenazine, trihexyphenidyl, thioridazine, thiothixene, trifluoperazine, or ziprasidone.

In some embodiments, the compound can be employed in combination with an anti-depressant or anti-anxiety agent, including norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, a-adrenoreceptor antagonists, neurokinin-1 receptor antagonists, atypical anti-depressants, benzodiazepines, 5-HT1A agonists or antagonists, especially 5-HT1A partial agonists, and corticotropin releasing factor (CRY) antagonists. Specific agents include: amitriptyline, clomipramine, doxepin, imipramine and trimipramine; amoxapine, desipramine, maprotiline, nortriptyline and protriptyline; fluoxetine, vortioxetine, fluvoxamine, paroxetine and sertraline; isocarboxazid, phenelzine, escitalopram, tranylcypromine and selegiline; venlafaxine, moclobemide; venlafaxine; duloxetine; aprepitant; bupropion, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone, and pharmaceutically acceptable salts thereof.

In some embodiments, the compounds can be coadministered with orthosteric muscarinic agonists, muscarinic potentiators, or cholinesterase inhibitors. In some embodiments, the compounds can be coadministered with GlyT1 inhibitors and the like such as, but not limited to: risperidone, quetiapine, clozapine, haloperidol, fluoxetine, prazepam, xanomeline, lithium, phenobarbitol, and salts thereof and combinations thereof.

f. Modes of Administration

Methods of treatment may include any number of modes of administering a disclosed composition. Modes of administration may include tablets, pills, dragees, hard and soft gel capsules, granules, pellets, aqueous, lipid, oily or other solutions, emulsions such as oil-in-water emulsions, liposomes, aqueous or oily suspensions, syrups, elixirs, solid emulsions, solid dispersions or dispersible powders. For the preparation of pharmaceutical compositions for oral administration, the agent may be admixed with commonly known and used adjuvants and excipients such as for example, gum arabic, talcum, starch, sugars (such as, e.g., mannitose, methyl cellulose, lactose), gelatin, surface-active agents, magnesium stearate, aqueous or non-aqueous solvents, paraffin derivatives, cross-linking agents, dispersants, emulsifiers, lubricants, conserving agents, flavoring agents (e.g., ethereal oils), solubility enhancers (e.g., benzyl benzoate or benzyl alcohol) or bioavailability enhancers (e.g. Gelucire™). In the pharmaceutical composition, the agent may also be dispersed in a microparticle, e.g. a nanoparticulate composition.

For parenteral administration, the agent can be dissolved or suspended in a physiologically acceptable diluent, such as, e.g., water, buffer, oils with or without solubilizers, surface-active agents, dispersants or emulsifiers. As oils for example and without limitation, olive oil, peanut oil, cottonseed oil, soybean oil, castor oil and sesame oil may be used. More generally spoken, for parenteral administration, the agent can be in the form of an aqueous, lipid, oily or other kind of solution or suspension or even administered in the form of liposomes or nano-suspensions.

The term “parenterally,” as used herein, refers to modes of administration which include intravenous, intramuscular, intraperitoneal, intrasternal, subcutaneous and intraarticular injection and infusion.

### 5. KITS

In one aspect, the invention provides kits comprising at least one compound of the invention or a pharmaceutically acceptable salt thereof, and one or more of:
(a) at least one agent known to increase mAChR M₄ activity;
(b) at least one agent known to decrease mAChR M₄ activity;
(c) at least one agent known to treat a disorder associated with cholinergic activity;
(d) instructions for treating a disorder associated with cholinergic activity;
(e) instructions for treating a disorder associated with M₄ receptor activity; or
(f) instructions for administering the compound in connection with cognitive or behavioral therapy.

In some embodiments, the at least one compound of the invention and the at least one agent are co-formulated. In some embodiments, the at least one compound of the invention and the at least one agent are co-packaged. The kits can also comprise compounds and/or products co-packaged, co-formulated, and/or co-delivered with other components. For example, a drug manufacturer, a drug reseller, a physician, a compounding shop, or a pharmacist can provide a kit comprising a compound of the invention and/or product and another component for delivery to a patient.

That the disclosed kits can be employed in connection with disclosed methods of use.

The kits may further comprise information, instructions, or both that use of the kit will provide treatment for medical conditions in mammals (particularly humans). The information and instructions may be in the form of words, pictures, or both, and the like. In addition or in the alternative, the kit may include the compound, a composition, or both; and information, instructions, or both, regarding methods of application of compound, or of composition, preferably with the benefit of treating or preventing medical conditions in mammals (e.g., humans).

The compounds and processes of the invention will be better understood by reference to the following examples, which are intended as an illustration of and not a limitation upon the scope of the invention.

### 6. EXAMPLES

All NMR spectra were recorded on a 400 MHz AMX Bruker NMR spectrometer. ¹H chemical shifts are reported in 6 values in ppm relative to residual protio-solvent. Data are reported as follows: chemical shift, multiplicity (s=singlet, bs=broad singlet, d=doublet, t=triplet, q=quartet, dd=doublet of doublets, m=multiplet, ABq=AB quartet), coupling constant, integration. Reversed-phase LCMS analysis was performed using an Agilent 1200 system comprised of a binary pump with degasser, high-performance autosampler, thermostated column compartment, C18 column, diode-array detector (DAD) and an Agilent 6150 MSD with the following parameters. The gradient conditions were 5% to 95% acetonitrile with the aqueous phase 0.1% TFA in water over 1.4 minutes. Samples were separated on a Waters Acquity UPLC BEH C18 column (1.7 μm, 1.0×50 mm) at 0.5 mL/min, with column and solvent temperatures maintained at 55° C. The DAD was set to scan from 190 to 300 nm, and the signals used were 220 nm and 254 nm (both with a band width of 4 nm). The MS detector was configured with an electrospray ionization source, and the low-resolution mass spectra were acquired by scanning from 140 to 700 AMU with a step size of 0.2 AMU at 0.13 cycles/second, and peak width of 0.008 minutes. The drying gas flow was set to 13 liters per minute at 300° C. and the nebulizer pressure was set to 30 psi. The capillary needle voltage was set at 3000 V, and the fragmentor voltage was set at 100V. Data acquisition was performed with Agilent Chemstation and Analytical Studio Reviewer software.

Abbreviations used in the examples and schemes that follows include the following: DCE is 1,2-dichloroethane; DCM is dichloromethane; DIEA is NN-diisopropylethylamine; DMF is N,N-dimethylformamide; DMSO is dimethyl sulfoxide; eq. is equivalent(s); EtOAc is ethyl acetate; FCC is flash column chromatography; h is hour(s); HATU is 1-[bis(dimethylamino)methylene]-1H-1,2,3-triazolo[4,5-b]pyridinium 3-oxid hexafluorophosphate; MeOH is methanol; NMP is N-methyl-2-pyrrolidone; rt and RT both refer to room temperature; MeCN is acetonitrile; min is minute(s); TFA is trifluoroacetic acid; CH(OEt)₃ is triethyl orthoformate; Boc₂O is di-tert-butyl decarbonate; dppf is 1,1′-bis(diphenylphosphanyl)ferrocene; Et₃N is triethylamine; 2-di-tert-butylphosphino-2′,4′,6′-triisopropylbiphenyl is t-BuXPhos; t-BuXPhos palladium(II) phenethylamine chloride is t-BuXPhos Palladacycle Gen-1; sodium tert-butoxide is NaOtBu; NCS is N-Chlorosuccinimide; sat. is saturated; and THE is tetrahydrofuran.

a. General Synthesis

Compounds of the invention may be prepared by synthetic processes or by metabolic processes. Preparation of the compounds by metabolic processes includes those occurring in the human or animal body (in vivo) or processes occurring in vitro.

Compounds of formula (I) may be synthesized as shown in General schemes I, II, III, IV, and V.

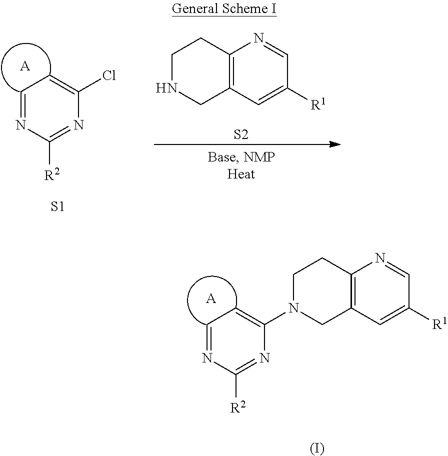

As shown in General Scheme I an intermediate of type S1 can react with an intermediate of type S2 to provide compounds of formula (I). Intermediates of type S2 may be synthesized according to general scheme II, III, IV or V depending on the R¹ group, or are commercially available such as 3-(trifluoromethyl)-5,6,7,8-tetrahydro-1,6-naphthyridine dihydrochloride (J&W Pharmlab); (CAS #870483-68-4). Reagents of type S1 are commercially available or can be prepared by methods known to the skilled person. The commercial reagents of type S1 include but are not limited to 4-chloro-3-methylisoxazolo[5,4-d]pyrimidine (Chembridge Corporation); (CAS #51850-60-3), 4-chloro-5-methylfuro[2,3-d]pyrimidine (Oxchem); (CAS #1321618-96-5), 4-chloro-6-methylthieno[2,3-d]pyrimidine (Chembridge Corporation); (CAS #106691-21-8), 4-chloro-5,6-dimethylthieno[2,3-d]pyrimidine (Alfa Aesar); (CAS #108831-68-1), 4-chloro-5-methylthieno[2,3-d]pyrimidine (Oakwood Products); (CAS #43088-67-1), 6-bromo-4-chlorothieno[2,3-d]pyrimidine (Combi-Blocks); (CAS #175137-21-0), 5-bromo-4-chlorothieno[2,3-d]pyrimidine (Synthonix, Inc.); (CAS #814918-95-1), 4-chlorothieno[2,3-d]pyrimidine (Combi-Blocks, Inc.); (CAS #14080-59-2), 4-chloro-6-isopropylthieno[2,3-d]pyrimidine (AstaTech, Inc.); (CAS #439692-52-1), 4-chloro-2,5,6-trimethylthieno[2,3-d]pyrimidine (Life Chemicals); (CAS #83548-58-7), 6-bromo-4-chlorothieno[2,3-d]pyrimidine (Combi-Blocks, Inc.); (CAS #56844-12-3), 6-bromo-4-chlorothieno[3,2-d]pyrimidine (D-L Chiral Chemicals); (CAS #31169-27-4), 4-chloro-7-methylthieno[3,2-d]pyrimidine (Combi-Blocks, Inc.); (CAS #175137-21-0), 4-chloro-5,6-dimethylthieno[2,3-d]pyrimidine (Alfa Aesar); (CAS #108831-68-1), 2,4-dichloro-6,7-dihydro-5H-cyclopenta[d]pyrimidine (Combi-Blocks, Inc.); (CAS #5466-43-3), 4-chlorothieno[3,2-d]pyrimidine (Oakwood Products, Inc.); (CAS #16269-66-2), 2,4-dichloro-5,7-dihydrofuro[3,4-d]pyrimidine (Astatech, Inc.); (CAS #848398-41-4).

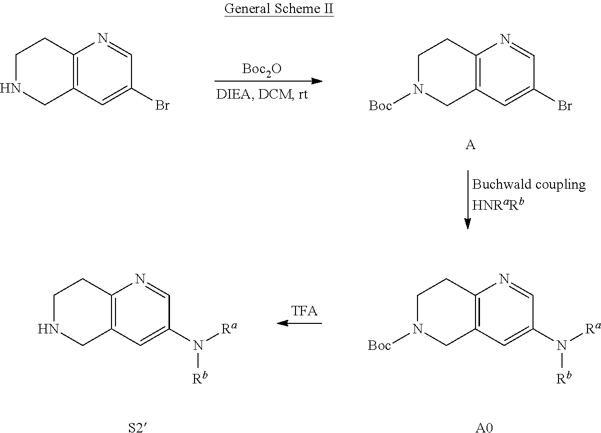

As shown in General Scheme II, intermediates of type S2′ can be prepared using a Buchwald coupling between intermediates such as A and an amine of type H1NR^{a}R^{b}. Intermediates like A0 can subsequently be deprotected using TFA to afford intermediates of type S2′.

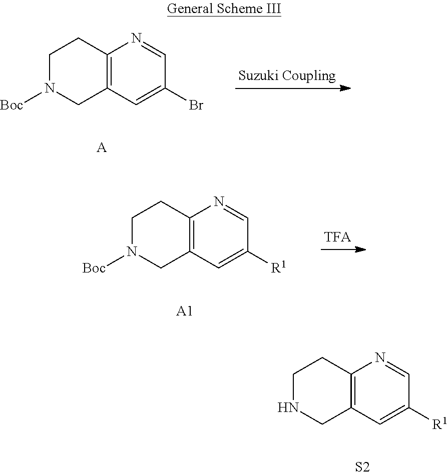

As shown in General Scheme III, intermediates of type S2, where R¹ is a 5- or 6-membered heteroaryl, can be prepared using a Suzuki coupling between A and a 5- or 6-membered heteroaryl. The intermediate A1 can subsequently be deprotected using TFA to afford intermediates of type S2.

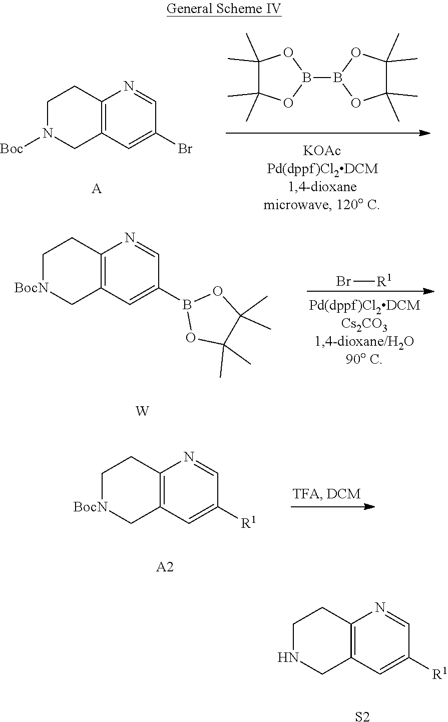

As shown in General Scheme IV, intermediates of type S2 where R¹ is benzyl are provided by reacting intermediate A with bis(pinacolato)diboron followed by a cross coupling reaction between intermediate W and a benzylbromide. The Boc-protected intermediate A2 may be deprotected using TFA.

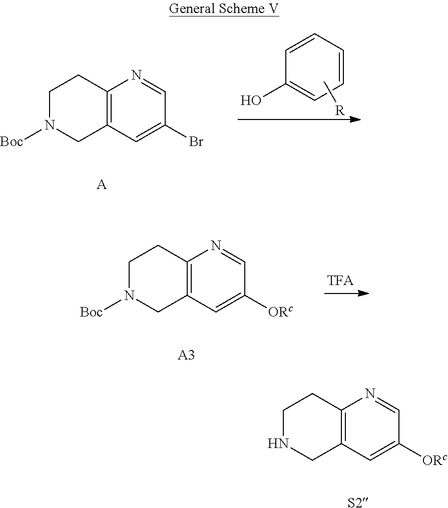

As shown in General Scheme V, intermediates of type S2″ where R¹ is a phenoxyl (OR^{c}) are provided by reacting a phenol with intermediate A to provide intermediate A3. The Boc-protected intermediate A3 may be deprotected using TFA.

The compounds of the invention and intermediates may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pre-treatment with activated carbon, thin-layer chromatography, distillation at various pressures, sublimation under vacuum, and trituration, as described for instance in “Vogel's Textbook of Practical Organic Chemistry”, 5th edition (1989), by Furniss, Hannaford, Smith, and Tatchell, pub. Longman Scientific & Technical, Essex CM20 2JE, England.

Reaction conditions and reaction times for each individual step can vary depending on the particular reactants employed and substituents present in the reactants used. Specific procedures are provided in the Examples section. Reactions can be worked up in the conventional manner, e.g. by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in PGM Wuts and TW Greene, in Greene's book titled Protective Groups in Organic Synthesis (4th ed.), John Wiley & Sons, NY (2006), which is incorporated herein by reference in its entirety. Synthesis of the compounds of the invention can be accomplished by methods analogous to those described in the synthetic schemes described hereinabove and in specific examples.

When an optically active form of a compound of the invention is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound is required, it can be obtained by carrying out one of the above procedures using a pure geometric isomer as a starting material, or by resolution of a mixture of the geometric isomers of the compound or intermediates using a standard procedure such as chromatographic separation.

It can be appreciated that the synthetic schemes and specific examples as described are illustrative and are not to be read as limiting the scope of the invention as it is defined in the appended claims. All alternatives, modifications, and equivalents of the synthetic methods and specific examples are included within the scope of the claims.

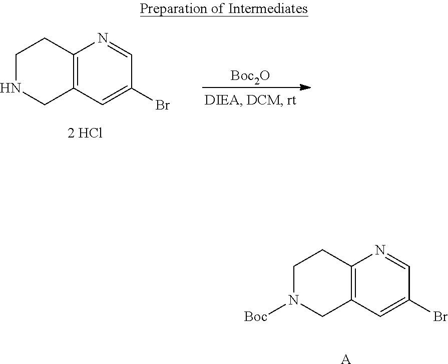

Tert-butyl 3-bromo-7,8-dihydro-5H-1,6-naphthyridine-6-carboxylate (Intermediate A): To a suspension of 3-bromo-5,6,7,8-tetrahydro-1,6-naphthyridine dihydrochloride [CAS #1187932-53-1, Aurum Pharmatech] (11.0 g) in DCM (165 mL) at rt was added di-tert-butyl dicarbonate (12.59 g) followed by a slow addition of N,N-diisopropylethylamine (26.7 mL). After stirring at rt for 16 h, the reaction mixture was concentrated under reduced pressure. The crude material was purified using flash chromatography on silica gel (0-50% EtOAc/hexanes) to provide the title compound (12.0 g) ¹H-NMR (400 MHz, DMSO-d₆) δ 8.48 (d, J=2.3 Hz, 1H), 7.94 (d, J=2.2 Hz, 1H), 4.54 (s, 2H), 3.64 (dd, J=6.0, 6.0 Hz, 2H), 2.82 (dd, J=6.0, 6.0 Hz, 2H), 1.42 (s, 9H); ES-MS [M+H]⁺=313.2/315.2.

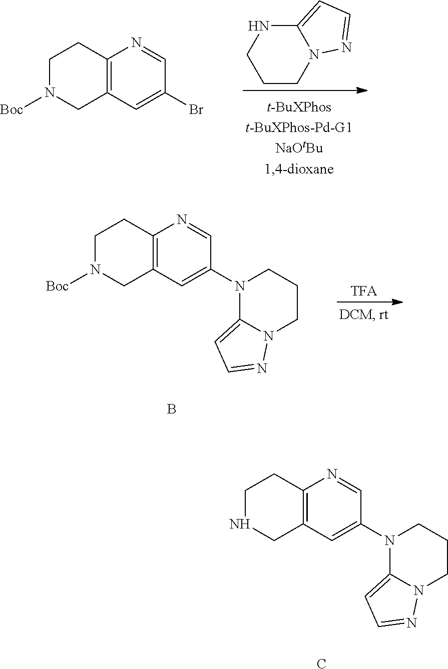

Tert-butyl 3-(6,7-dihydro-5H-pyrazolo[1,5-a]pyrimidin-4-yl)-7,8-dihydro-5H-1,6-naphthyridine-6-carboxylate (Intermediate B): Tert-butyl 3-bromo-7,8-dihydro-5H-1,6-naphthyridine-6-carboxylate (939.6 mg), 4,5,6,7-tetrahydropyrazolo[1,5-a]pyrimidine (554.2 mg), t-BuXPhos (191.1 mg), t-BuXPhos Palladacycle Gen-1 (309.5 mg) and sodium tert-butoxide (576.62 mg) were charged into a reaction vial. The mixture was evacuated and purged with nitrogen and degassed 1,4-dioxane (5.0 mL) and tert-butyl alcohol (15.0 mL) were added. The reaction mixture was allowed to stir at 100° C. for 2 h. After cooling at rt, the mixture was filtered through a pad of Celite® which was rinsed thoroughly with EtOAc and DCM. The filtrate was concentrated under reduced pressure. The crude material was purified using RP-HPLC (method, 50×250 mm column, 10-50% MeCN in 0.1% TFA aqueous solution). The fractions were concentrated to provide the title compound (1.15 g). ¹H-NMR (400 MHz, CDCl₃) δ 8.58 (br s, 1H), 7.85 (d, J=2.2 Hz, 1H), 7.49 (d, J=2.3 Hz, 1H), 5.83 (d, J=2.4 Hz, 1H), 4.70 (s, 2H), 4.36 (dd, J=6.2, 6.2 Hz, 2H), 3.80 (dd, J=5.7, 5.7 Hz, 4H), 3.24 (dd, J=5.9, 5.9 Hz, 2H), 2.39 (ddd, J=11.9, 6.1, 6.1 Hz, 2H), 1.50 (s, 9H); ES-MS [M+H]⁺=356.4.

Tert-butyl 3-(6,7-dihydro-5H-pyrazolo[1,5-a]pyrimidin-4-yl)-7,8-dihydro-5H-1,6-naphthyridine-6-carboxylate (Intermediate C): To a solution of tert-butyl 3-(6,7-dihydro-5H-pyrazolo[1,5-a]pyrimidin-4-yl)-7,8-dihydro-5H-1,6-naphthyridine-6-carboxylate (1.15 g, 2.45 mmol, 1.0 eq.) in DCM (12.5 mL, 0.2 M) was added trifluoroacetic acid (1.87 mL, 24.5 mmol, 10.0 eq.). The reaction mixture was allowed to stir at rt for 2 h then concentrated under reduced pressure. The crude material was suspended in THE (10 mL) and a HCl solution (4 M in 1,4-dioxane, 6.0 mL) was added. The resulting suspension was allowed to stir at rt for 30 min then solvent was removed under reduced pressure. The resulting solid was dried under vacuum to provide the title compound as a HCl salt (0.80 g). The material was carried forward without any further purification. ¹H-NMR (400 MHz, DMSO-d₆) δ 9.94 (br s, 1H), 8.58 (d, J=2.6 Hz, 1H), 7.97 (d, J=1.8 Hz, 1H), 7.52 (d, J=2.2 Hz, 1H), 5.94 (d, J=2.3 Hz, 1H), 4.36 (dd, J=3.2, 3.2 Hz, 2H), 4.18 (dd, J=6.1, 6.1 Hz, 2H), 3.78 (dd, J=5.4, 5.4 Hz, 4H), 3.49-3.46 (m, 2H), 3.20 (dd, J=6.1, 6.1 Hz, 2H), 2.24 (ddd, J=11.5, 5.9, 5.9 Hz, 2H); ES-MS [M+H]⁺=256.2.

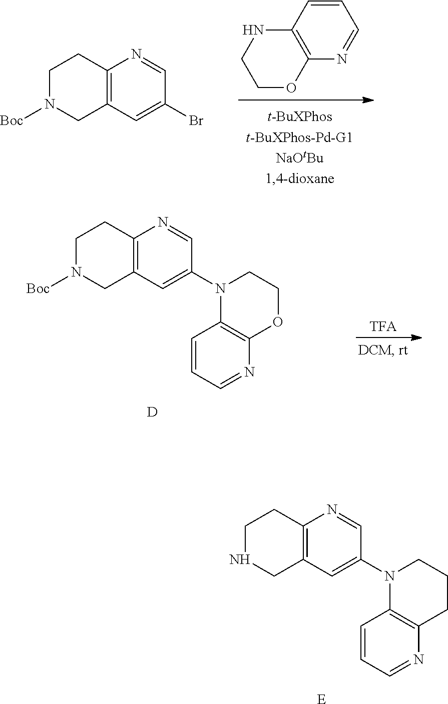

Tert-butyl 3-(2,3-dihydropyrido[2,3-b][1,4]oxazin-1-yl)-7,8-dihydro-5H-1,6-naphthyridine-6-carboxylate (Intermediate D): Prepared in a similar manner as Intermediate B to provide the title compound (1.15 g). ¹H-NMR (400 MHz, DMSO-d₆) δ 8.50 (d, J=2.5 Hz, 1H), 7.90 (d, J=2.2 Hz, 1H), 7.70 (dd, J=4.8, 1.6 Hz, 1H), 7.29 (dd, J=7.9, 1.4 Hz, 1H), 6.87 (dd, J=8.0, 4.8 Hz, 1H), 4.60 (s, 2H), 4.47-4.43 (m, 2H), 3.77 (dd, J=4.4, 4.4 Hz, 2H), 3.68 (dd, J=5.9, 5.9 Hz, 2H), 2.94 (dd, J=5.9, 5.9 Hz, 2H), 1.43 (s, 9H); ES-MS [M+H]=369.3.

1-(5,6,7,8-Tetrahydro-1,6-naphthyridin-3-yl)-2,3-dihydropyrido[2,3-b][1,4]oxazine (Intermediate E): Prepared in a similar manner as Intermediate C to provide the title compound (HCl salt; 0.73 g). The material was carried forward without further purification. ¹H-NMR (400 MHz, DMSO-d₆) δ 9.89 (br s, 1H), 8.56 (d, J=2.5 Hz, 1H), 7.91 (d, J=2.0 Hz, 1H), 7.74 (dd, J=5.0, 1.6 Hz, 1H), 7.37 (dd, J=8.0, 1.4 Hz, 1H), 6.95 (dd, J=8.0, 5.0 Hz, 1H), 4.50 (dd, J=4.1, 4.1 Hz, 2H), 4.33 (s, 2H), 3.81 (dd, J=4.4, 4.4 Hz, 2H), 3.51-3.44 (m, 2H), 3.19 (dd, J=6.1, 6.1 Hz, 2H); ES-MS [M+H]⁺=269.4.

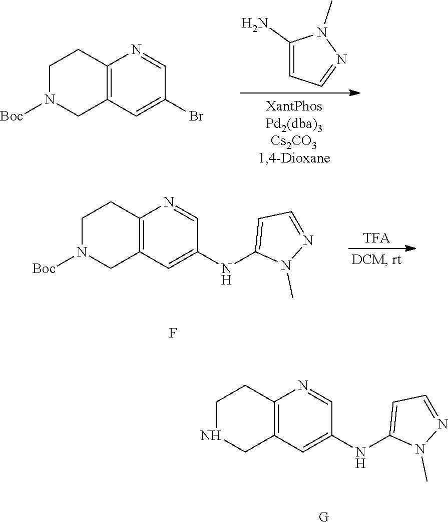

Tert-butyl 3-[(2-methylpyrazol-3-yl)amino]-7,8-dihydro-5H-1,6-naphthyridine-6-carboxylate (Intermediate F): Tert-butyl 3-bromo-7,8-dihydro-5H-1,6-naphthyridine-6-carboxylate (940 mg), 5-amino-1-methylpyrazole (582 mg), tris(dibenzylideneacetone)dipalladium(0) (275 mg), XantPhos (260 mg) and cesium carbonate (1.97 g) were charged into a reaction vial. The mixture was evacuated and purged with nitrogen then degassed 1,4-dioxane (20 mL, 0.15 M) was added. After stirring at 100° C. for 3 h, the reaction mixture was cooled to rt and filtered through a pad of Celite® which was rinsed thoroughly with EtOAc. The filtrate was concentrated and the crude material was purified using flash column chromatography on silica gel (0-10% MeOH/DCM). The material was then further purified using RP-HPLC (10-50% MeCN in 0.1% TFA aqueous solution) to provide the title compound as the TFA salt (1.45 g). ¹H-NMR (400 MHz, DMSO-d₆) δ 8.44 (s, 1H), 8.06 (d, J=2.6 Hz, 1H), 7.40 (d, J=2.0 Hz, 1H), 7.24 (broad s, 1H), 6.10 (d, J=1.9 Hz, 1H), 4.52 (s, 2H), 3.65 (s, 3H), 3.62 (dd, J=6.0, 6.0 Hz, 2H), 2.83 (dd, J=5.9, 5.9 Hz, 2H), 1.42 (s, 9H); ES-MS [M+H]⁺=330.4.

N-(2-methylpyrazol-3-yl)-5,6,7,8-tetrahydro-1,6-naphthyridin-3-amine (Intermediate G): Prepared in a similar manner as Intermediate C to provide the title compound as the HCl salt. The material was carried forward without further purification. ¹H-NMR (400 MHz, DMSO-d₆) δ 9.81 (broad s, 1H), 9.18 (s, 1H), 8.22 (d, J=2.6 Hz, 1H), 7.53 (d, J=1.7 Hz, 1H), 7.47 (d, J=2.0 Hz, 1H), 6.18 (d, J=2.0 Hz, 1H), 4.34 (s, 2H), 3.70 (s, 3H), 3.47-3.44 (m, 2H), 3.17 (dd, J=6.1, 6.1 Hz, 2H); ES-MS [M+H]⁺=230.2.

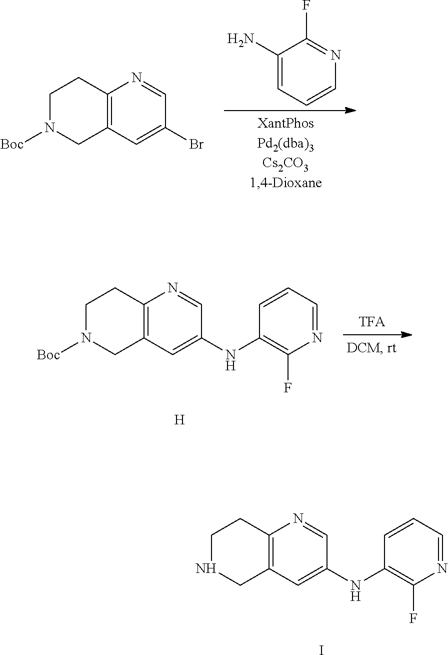

Tert-butyl 3-[(2-fluoro-3-pyridyl)amino]-7,8-dihydro-5H-1,6-naphthyridine-6-carboxylate (Intermediate H): Prepared in a similar manner as Intermediate F. The material was then further purified using RP-HPLC (15-55% MeCN in 0.05% NH₄OH aqueous solution) to provide the title compound. ¹H-NMR (400 MHz, DMSO-d₆) δ 8.28 (s, 1H), 8.20 (d, J=2.6 Hz, 1H), 7.71-7.67 (m, 2H), 7.31 (d, J=1.8 Hz, 1H), 7.21 (dddd, J=7.7, 4.6, 1.0, 1.0, 1H), 4.49 (s, 2H), 3.64 (dd, J=5.9, 5.9 Hz, 2H), 3.31 (s, 3H), 2.80 (dd, J=5.9, 5.9 Hz, 2H), 1.43 (s, 9H); ES-MS [M+H]⁺=345.4.

N-(2-fluoro-3-pyridyl)-5,6,7,8-tetrahydro-1,6-naphthyridin-3-amine (Intermediate I): Prepared in a similar manner as Intermediate C to provide the title compound as the HCl salt. The material was carried forward without further purification. ¹H-NMR (400 MHz, DMSO-d₆) δ 9.81 (br s, 1H), 9.03 (s, 1H), 8.32 (d, J=2.6 Hz, 1H), 7.88-7.81 (m, 2H), 7.67 (d, J=1.6 Hz, 1H), 7.32 (dddd, J=7.7, 4.8, 1.2, 1.2 Hz, 1H), 4.33 (s, 2H), 3.56 (s, 3H), 3.49-3.43 (m, 2H), 3.17 (dd, J=6.1, 6.1 Hz, 2H); ES-MS [M+H]⁺=245.2.

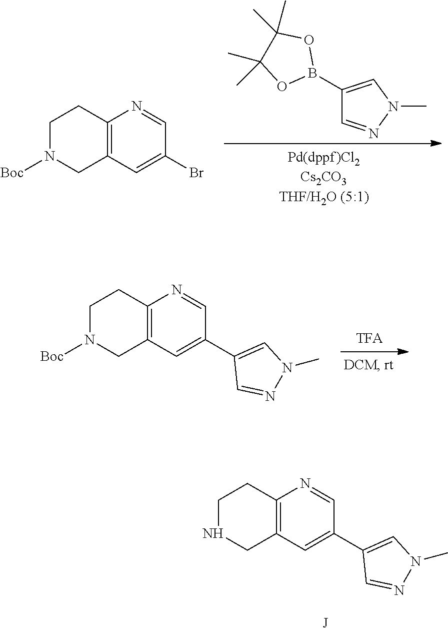

3-(1-methylpyrazol-4-yl)-5,6,7,8-tetrahydro-1,6-naphthyridine (Intermediate J): Tert-butyl 3-bromo-7,8-dihydro-5H-1,6-naphthyridine-6-carboxylate (1.0 g), 1-methylpyrazole-4-boronic acid pinacol ester (830 mg), Pd(dppf)Cl₂ (350 mg), cesium carbonate (3.14 g), 1,4-dioxane (15.0 mL), and water (3.0 mL) were charged into a reaction vial which was then purged with N₂. After 18 h at 100° C., the mixture was cooled to rt and filtered through a pad of Celite® which was washed thoroughly with EtOAc and DCM. The filtrate was concentrated under reduced pressure. The crude material was re-dissolved in DCM:H₂O (5:1 v/v, 50 mL). The solution was passed through a phase separator and the organic layer was concentrated. The crude material was purified by RP-HPLC (10-50% MeCN in 0.1% TFA aqueous solution) to provide the Boc-protected intermediate. ES-MS [M+H]=315.2. The material was re-dissolved in DCM (14.0 mL) and TFA (14.0 mL) was added. After 1 h at rt, the mixture was concentrated under reduced pressure. The crude material was suspended in a HCl solution (4M in 1,4-dioxane, 20 mL) for 15 min. The solvent was removed to yield the title compound as the HCl salt (575 mg). ¹H-NMR (400 MHz, DMSO-d₆) δ 9.91 (s, 1H), 8.92 (d, J=1.7 Hz, 1H), 8.37 (s, 1H), 8.30 (s, 1H), 8.05 (s, 1H), 4.38 (s, 2H), 3.90 (s, 3H), 3.49 (d, J=4.2 Hz, 2H), 3.26 (dd, J=6.0, 6.0 Hz, 2H); ES-MS [M+H]⁺=215.2.

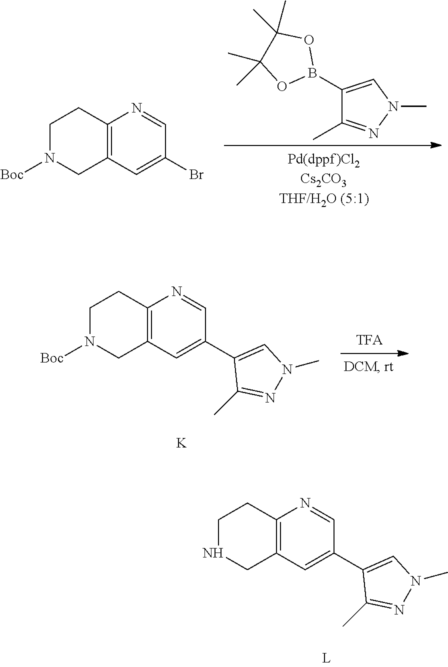

Tert-butyl 3-(1,3-dimethylpyrazol-4-yl)-7,8-dihydro-5H-1,6-naphthyridine-6-carboxylate (Intermediate K): Tert-butyl 3-bromo-7,8-dihydro-5H-1,6-naphthyridine-6-carboxylate (1.25 g), cesium carbonate (3.91 g), Pd(dppf)Cl₂ (293.5 mg) and 1,3-dimethylpyrazole-4-boronic acid pinacol ester (1.33 g) were added into a reaction vial which was then evacuated and purged with nitrogen. 1,4-dioxane (16.6 mL, 0.2 M) and water (3.33 mL) were added. After stirring 2 h at 100° C., the reaction mixture was cooled to rt and filtered through a pad of Celite® which was rinsed thoroughly with EtOAc and DCM. The filtrate was concentrated and the crude material was purified using RP-HPLC (10-50% MeCN in 0.1% TFA aqueous solution) to provide the desired product as the TFA salt (1.54 g); ES-MS [M+H]⁺=329.4.

3-(1,3-dimethylpyrazol-4-yl)-5,6,7,8-tetrahydro-1,6-naphthyridine (Intermediate L): Prepared in a similar manner as Intermediate C to provide the title compound as a HCl salt (0.98 g). The material was carried forward without further purification. ¹H-NMR (400 MHz, DMSO-d₆) δ 10.0 (br s, 1H), 8.74 (d, J=1.9 Hz, 1H), 8.2 (s, 1H), 8.17 (s, 1H), 4.42 (s, 2H), 3.82 (s, 3H), 3.51-3.47 (m, 2H), 3.30 (dd, J=6.1, 6.1 Hz, 2H), 2.35 (s, 3H); ES-MS [M+H]⁺=229.2.

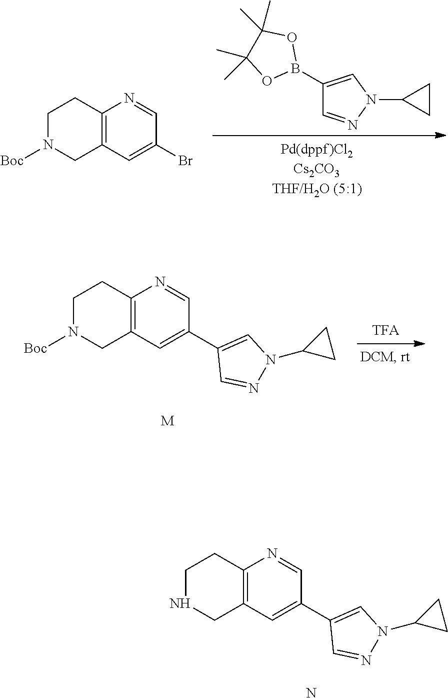

Tert-butyl 3-(1-cyclopropylpyrazol-4-yl)-7,8-dihydro-5H-1,6-naphthyridine-6-carboxylate (Intermediate M): Prepared in a similar manner as Intermediate K to provide the title compound. ES-MS [M+H]⁺=341.4.

3-(1,3-dimethylpyrazol-4-yl)-5,6,7,8-tetrahydro-1,6-naphthyridine (Intermediate N): Prepared in a similar manner as Intermediate C to provide the title compound as the HCl salt. The material was carried forward without further purification. ¹H-NMR (400 MHz, DMSO-d₆) δ 9.86 (br s, 1H), 8.92 (d, J=1.7 Hz, 1H), 8.49 (s, 1H), 8.28 (s, 1H), 8.03 (s, 1H), 4.37 (s, 2H), 3.81-3.75 (m, 1H), 3.49 (dd, J=5.5, 5.5 Hz, 2H), 3.24 (dd, J=6.1, 6.1 Hz, 2H), 1.09-0.99 (m, 4H); ES-MS [M+H]⁺=241.2.

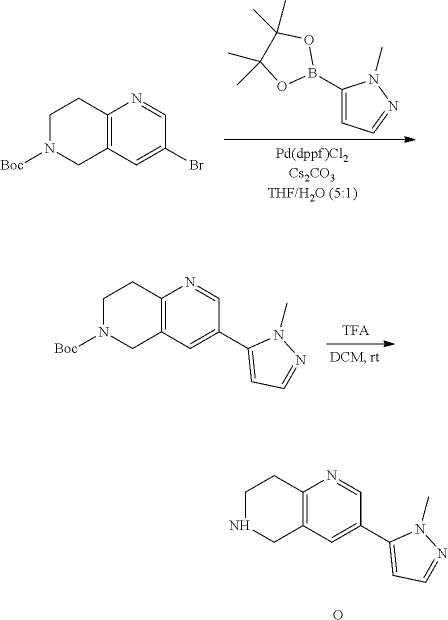 [SMILES: CC(C)(C)OC(=O)N1CCc2ncc(Br)cc2C1 ; Cn1nccc1B1OC(C)(C)C(C)(C)O1 ; Cn1nccc1-c1cnc2c(c1)CN(C(=O)OC(C)(C)C)CC2 ; Cn1nccc1-c1cnc2c(c1)CNCC2 ; O]

3-(1-methyl-1H-pyrazol-5-yl)-5,6,7,8-tetrahydro-1,6-naphthyridine (Intermediate O): Prepared in a similar manner as in Intermediate J to provide the title compound. ¹H-NMR (400 MHz, DMSO-d₆) δ 9.35 (br, s), 8.67 (d, J=22 Hz, 1H), 7.91 (d, J=2.1 Hz, 1H), 7.53 (d, J=1.9 Hz, 1H), 6.52 (d, J=2.9 Hz, 1H), 4.38 (dd, J=4.4, 4.4 Hz, 2H), 3.88 (s, 3H), 3.55-3.52 (m, 2H), 3.14 (dd, J=6.3, 6.3 Hz, 2H); ES-MS [M+H]⁺=215.2.

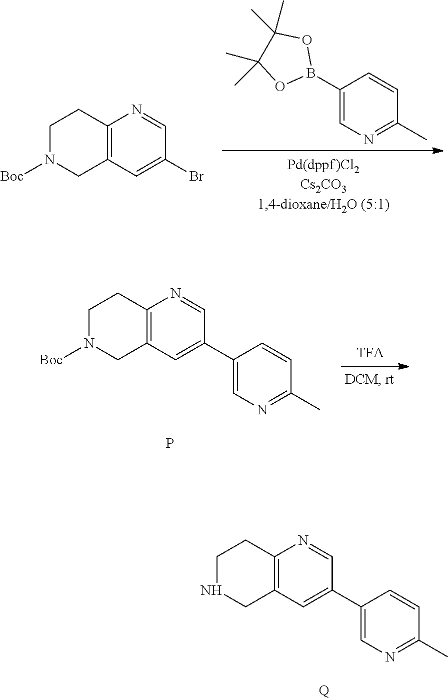

Tert-butyl 3-(6-methyl-3-pyridyl)-7,8-dihydro-5H-1,6-naphthyridine-6-carboxylate (Intermediate P): Prepared in a similar manner as Intermediate K to provide the title compound as the TFA salt; ES-MS [M+H]⁺=326.4.

3-(6-methyl-3-pyridyl)-5,6,7,8-tetrahydro-1,6-naphthyridine (Intermediate Q): Prepared in a similar manner as Intermediate C to provide the title compound as the HCl salt. The material was carried forward without further purification. ¹H-NMR (400 MHz, DMSO-d₆) δ 9.97 (br s, 1H), 9.19 (d, J=1.7 Hz, 1H), 9.03 (d, J=1.8 Hz, 1H), 8.81 (dd, J=8.3, 1.4 Hz, 1H), 8.35 (s, 1H), 8.02 (d, J=8.4 Hz, 1H), 4.39 (s, 2H), 3.56-3.46 (m, 1H), 3.23 (dd, J=6.0, 6.0 Hz, 2H), 2.80 (s, 3H); ES-MS [M+H]⁺=226.2.

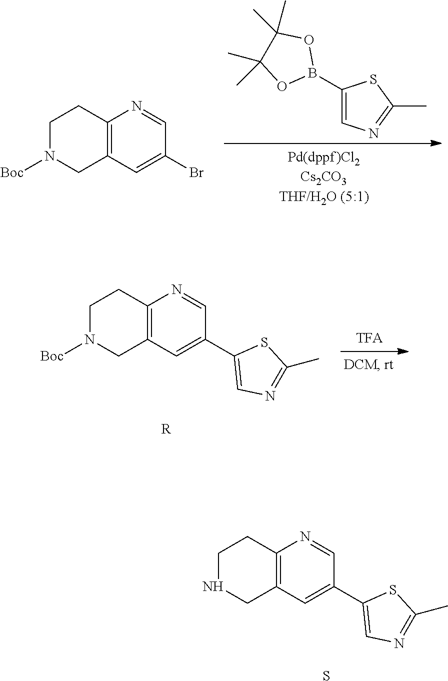

Tert-butyl 3-(2-methylthiazol-5-yl)-7,8-dihydro-5H-1,6-naphthyridine-6-carboxylate (Intermediate R): Prepared in a similar manner as Intermediate K to provide the title compound as the TFA salt; ES-MS [M+H]⁺=332.4. ¹H-NMR (400 MHz, DMSO-d₆) δ 8.63 (d, J=2.3 Hz, 1H), 8.11 (s, 1H), 7.89 (d, J=2.1 Hz, 1H), 4.59 (s, 2H), 3.67 (dd, J=5.9, 5.9 Hz, 2H), 2.88 (dd, J=5.9, 5.9 Hz, 2H), 2.70 (s, 3H), 1.44 (s, 9H); ES-MS [M+H]⁺=226.2.

2-methyl-5-(5,6,7,8-tetrahydro-1,6-naphthyridin-3-yl)thiazole (Intermediate S): Prepared in a similar manner as Intermediate C to provide the title compound as the HCl salt. The material was carried forward without further purification. ¹H-NMR (400 MHz, DMSO-d₆) δ 9.95 (br s, 1H), 8.88 (d, J=2.1 Hz, 1H), 8.22 (s, 1H), 8.14 (d, J=1.8 Hz, 1H), 4.36 (s, 2H), 3.49-3.47 (m, 2H), 3.21 (dd, J=6.2, 6.2 Hz, 2H), 2.72 (s, 3H); ES-MS [M+H]⁺=223.3.

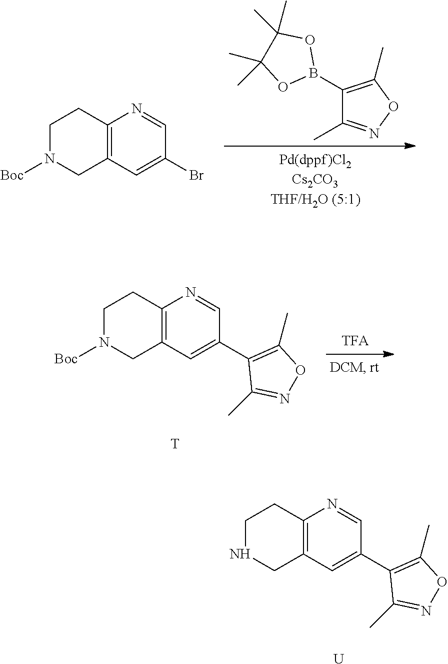

Tert-butyl 3-(3,5-dimethylisoxazol-4-yl)-7,8-dihydro-5H-1,6-naphthyridine-6-carboxylate (Intermediate T): Prepared in a similar manner as Intermediate K to provide the title compound as the TFA salt. ¹H-NMR (400 MHz, DMSO-d₆) δ 8.52 (d, J=1.9 Hz, 1H), 7.91 (d, J=1.6 Hz, 1H), 4.65 (s, 2H), 3.70 (dd, J=5.9, 5.9 Hz, 2H), 2.97 (dd, J=5.9, 5.9 Hz, 2H), 243 (s, 3H), 2.25 (s, 3H), 1.44 (s, 9H); ES-MS [M+H]⁺=330.4.

3,5-dimethyl-4-(5,6,7,8-tetrahydro-1,6-naphthyridin-3-yl)isoxazole (Intermediate U): Prepared in a similar manner as Intermediate C to provide the title compound as the HCl salt. The material was carried forward without further purification. ¹H-NMR (400 MHz, DMSO-d₆) δ 9.73 (br s, 1H), 8.58 (d, J=2.0 Hz, 1H), 7.87 (d, J=1.8 Hz, 1H), 4.3 (dd, J=4.1, 4.1 Hz, 2H), 3.51-3.46 (m, 2H), 3.20 (dd, J=6.2, 6.2 Hz, 2H), 2.43 (s, 3H), 2.24 (s, 3H); ES-MS [M+H]⁺=230.2.

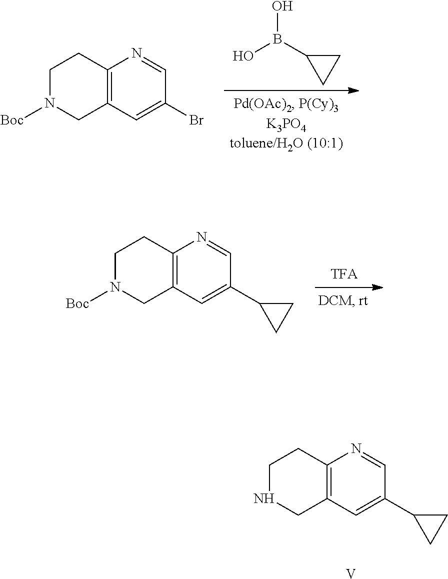

3-cyclopropyl-5,6,7,8-tetrahydro-1,6-naphthyridine dihydrochloride (Intermediate V): Tert-butyl 3-bromo-7,8-dihydro-1,6-naphthyridine-6(5H)-carboxylate (1.0 g), cyclopropylboronic acid (549 mg), tricyclohexylphosphine (179 mg), palladium(II) acetate (72.3 mg), and tribasic potassium phosphate (2.06 g) were combined in a reaction vial which was purged with nitrogen. Toluene (15 mL) and water (1.5 mL) were added. After stirring at 110° C. for 4 h, the reaction mixture was concentrated under reduced pressure. The crude residue was diluted in DMSO (10 mL) and passed through a syringe filter to remove any insoluble salts. The filtrate was purified using RP-HPLC (10-50% MeCN in 0.1% TFA aqueous solution) to afford the Boc-protected intermediate. ES-MS [M+H]⁺=275.2. The material was then suspended in DCM (15.0 mL) and trifluoroacetic acid (3.0 mL) was added dropwise. After 2 h at rt, the reaction mixture was concentrated under reduced pressure. The crude material was suspended in a HCl solution (4M in 1,4-dioxane, 20 mL) for 15 min, and the solvent was removed to yield the title compound as the HCl salt (766 mg). ¹H-NMR (400 MHz, DMSO-d₆) δ 9.88 (s, 1H), 8.56 (s, 1H), 7.83 (s, 1H), 4.32 (s, 2H), 3.51-3.45 (m, 2H), 3.24 (dd, J=6.1, 6.1 Hz 3H), 2.09 (dddd, J=8.4, 8.4, 5.0, 5.0 Hz, 1H), 1.09 (dddd, J=8.4, 4.7, 4.7, 4.7 Hz, 2H), 0.83 (dddd, J=4.9, 4.7, 4.7, 4.7 Hz, 2H); ES-MS [M+H]⁺=175.4.

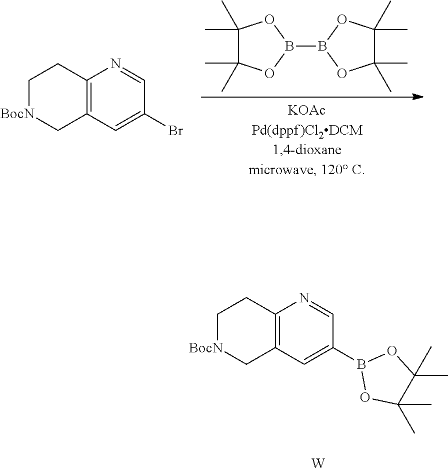

Tert-butyl 3-(4,4,5,5-tetramethyl-1,3,2-dioxaborolan-2-yl)-7,8-dihydro-1,6-naphthyridine-6(5H)-carboxylate (Intermediate W): Tert-butyl 3-bromo-7,8-dihydro-1,6-naphthyridine-6(5H)-carboxylate (882 mg), bis(pinacolato)diboron (1.43 g), potassium acetate (829 mg) and Pd(dppf)Cl₂ DCM (231 mg) were combined in a microwave vial which was sealed and placed under an inert atmosphere. 1,4-dioxane (26 mL) was added via syringe, and the resulting mixture was heated under microwave irradiation at 120° C. for 30 min, after which time the reaction mixture was cooled to rt and filtered through a plug of Celite® with DCM and EtOAc. Solvents were concentrated under reduced pressure to give the title compound (1.01 g), which was used directly without further purification. ES-MS [M+H]⁺=279.2 (mass of boronic acid is observed).

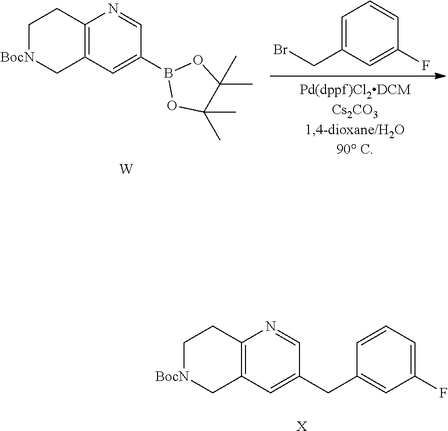

tert-butyl 3-(3-fluorobenzyl)-7,8-dihydro-1,6-naphthyridine-6(5H)-carboxylate (Intermediate X): Tert-butyl 3-(4,4,5,5-tetramethyl-1,3,2-dioxaborolan-2-yl)-7,8-dihydro-1,6-naphthyridine-6(5H)-carboxylate (254 mg), 3-fluorobenzyl bromide (200 mg), cesium carbonate (692 mg) and Pd(dppf)Cl₂ DCM (86 mg) were combined in a sealed vial and placed under an inert atmosphere. A 1,4-dioxane/H2O solution (5:1, 5 mL, degassed) was added via syringe, and the resulting mixture was stirred at 90° C. for 1 h, after which time the reaction mixture was cooled to rt, and the solvents were removed under reduced pressure. The crude residue was taken up in DCM, and solids were removed by syringe filtration. The crude residue was purified by column chromatography (3-50% EtOAc in hexanes) to give the title compound (143 mg). ES-MS [M+H]⁺=343.4.

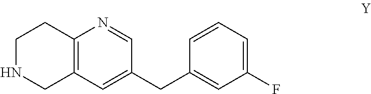 [SMILES: Fc1cccc(Cc2cnc3c(c2)CNCC3)c1]

3-(3-fluorobenzyl)-5,6,7,8-tetrahydro-1,6-naphthyridine (Intermediate Y): Tert-butyl 3-(3-fluorobenzyl)-7,8-dihydro-1,6-naphthyridine-6(5H)-carboxylate (138 mg) was dissolved in DCM (2 mL), and trifluoroacetic acid (0.31 mL) was added dropwise. The resulting solution was stirred at rt for 1 h, after which time the solvents were concentrated, and the residue was taken up in sat. NaHCO₃. The aqueous layer was extracted with 3:1 chloroform/IPA (v/v), and organic extracts were filtered through a phase separator and concentrated to give the title compound (92 mg). ¹H-NMR (400 MHz, CDCl₃) δ 8.42 (s, 1H), 7.32 (d, J=2.0 Hz, 1H), 7.27 (dd, J=7.9, 1.9 Hz, 1H), 6.97 (d, J=7.1 Hz, 1H), 6.94 (ddd, J=8.5, 8.6, 2.5 Hz, 1H), 6.87 (ddd, J=1.8, 11.6, 3.8 Hz, 1H), 3.98 (s, 4H), 2.95 (dd, J=6.4, 6.2 Hz, 2H), 1.64 (bs, J=1.6 Hz, 2H); ES-MS [M+H]⁺=243.6.

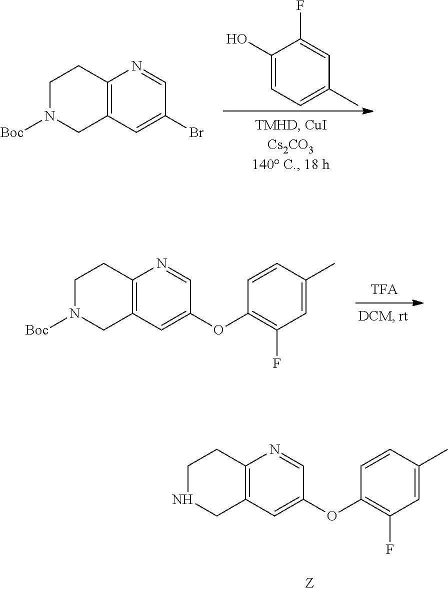

3-(2-fluoro-4-methyl-phenoxy)-5,6,7,8-tetrahydro-1,6-naphthyridine (Intermediate Z): Tert-butyl 3-bromo-7,8-dihydro-5H-1,6-naphthyridine-6-carboxylate (800 mg), 2-fluoro-4-methylphenol (644 mg), cesium carbonate (1.67 g), 2,2,6,6-tetramethyl-3,5-heptanedione (TMHD) (54 μL), copper(I) iodide (24 mg), and NMP (12 mL) were combined. The reaction vessel was sealed and purged with N₂. After 18 h at 140° C., the reaction mixture was cooled to rt and filtered through a pad of Celite®. The filtrate was concentrated under reduced pressure and the residue was purified using RP-HPLC (10-50% MeCN in 0.1% TFA aqueous solution) to afford the Boc-protected intermediate. The intermediate was dissolved in DCM (10 mL) and TFA (10 mL). After stirring at rt for 2 h, the reaction mixture was concentrated under reduced pressure. Excess TFA was removed using HF-SCX cartridge (10 g) and NH₃ solution (2 M in MeOH) to provide the title compound (330 mg). ¹H-NMR (400 MHz, CDCl₃) δ 8.19 (d, J=2.8 Hz, 1H), 6.99 (dd, J=1.4, 13 Hz, 1H), 6.94 (d, J=7.8 Hz, 1H), 6.92 (m, J=17.0, 1H), 6.85 (d, J=2.5 Hz, 1H), 3.97 (s, 1H), 3.23 (t, J=6.4 Hz, 2H), 2.93 (t, J=6.0 Hz, 2H), 2.35 (s, 3H); ES-MS [M+H]⁺=243.6.

b. Muscarinic Acetylcholine Receptor M4 Activity

In some embodiments, the compounds of the invention potentiate the agonist response (e.g., acetylcholine) of mAChR M₄. In some embodiments, the compounds of the invention increase mAChR M₄ response to non-maximal concentrations of agonist in the presence of compound compared to the response to agonist in the absence of compound. The potentiation of mAChR M₄ activity can be demonstrated by methodology known in the art. For example, activation of mAChR M₄ activity can be determined by measurement of calcium flux in response to an agonist, e.g. acetylcholine, in cells loaded with a Ca²⁺-sensitive fluorescent dye (e.g., Fluo-4) and co-expression of a chimeric or promiscuous G protein. In some embodiments, the calcium flux was measured as an increase in fluorescent static ratio. In some embodiments, positive allosteric modulator activity was analyzed as a concentration-dependent increase in the EC₂₀ acetylcholine response (i.e. the response of mAChR M₄ at a concentration of acetylcholine that yields 20% of the maximal response).

In an embodiment, a compound of the invention activate mAChR M4 response as an increase in calcium fluorescence in mAChR M₄-transfected CHO-K1 cells in the presence of the compound, compared to the response of equivalent CHO-K1 cells in the absence of the compound. In an embodiment, a compound of the invention activates the mAChR M₄ response with an EC₅₀ of less than about 10 μM, less than about 5 μM, less than about 1 μM, less than about 500 nM, of less than about 100 nM, or less than about 50 nM. In some embodiments, the mAChR M₄-transfected CHO-K1 cells are transfected with human mAChR M₄. In an embodiment, the mAChR M₄-transfected CHO-K1 cells are transfected with rat mAChR M₄.

The compounds of the invention may exhibit positive allosteric modulation of mAChR M₄ response to acetylcholine as an increase in response to non-maximal concentrations of acetylcholine in CHO-K1 cells transfected with a mAChR M₄ in the presence of the compound, compared to the response to acetylcholine in the absence of the compound. In some embodiments, the compounds of the invention exhibit positive allosteric modulation of the mAChR M₄ response to acetylcholine with an EC₅₀ of less than about 10 μM, less than about 5 μM, less than about 1 μM, less than about 500 nM, or less than about 100 nM. In some embodiments, the EC₅₀ for positive allosteric modulation is determined in CHO-K1 cells that are transfected with a mAChR M₄. In some embodiments, the mAChR M₄ transfected is human mAChR M₄. In some embodiments, the mAChR M₄ transfected is rat mAChR M₄.

The compounds of the invention may activate mAChR M₄ response in mAChR M₄-transfected CHO-K1 cells with an EC₅₀ less than the EC₅₀ for one or more of mAChR M₁, M₂, M₃ or M₅-transfected CHO-K1 cells. That is, a compound of the invention can have selectivity for the mAChR M₄ receptor vis-à-vis one or more of the mAChR M₁, M₂, M₃ or M₅ receptors. For example, in some embodiments, a compound of the invention can activate mAChR M₄ response with an EC₅₀ of about 5-fold less, about 10-fold less, about 20-fold less, about 30-fold less, about 50-fold less, about 100-fold less, about 200-fold less, about 300-fold less, about 400-fold less, or greater than about 500-fold less than that for mAChR M₁. In some embodiments, a compound of the invention can activate mAChR M₄ response with an EC₅₀ of about 5-fold less, about 10-fold less, about 20-fold less, about 30-fold less, about 50-fold less, about 100-fold less, about 200-fold less, about 300-fold less, about 400-fold less, or greater than about 500-fold less than that for mAChR M₂. In some embodiments, a compound of the invention can activate mAChR M₄ response with an EC₅₀ of about 5-fold less, about 10-fold less, about 20-fold less, about 30-fold less, about 50-fold less, about 100-fold less, about 200-fold less, about 300-fold less, about 400-fold less, or greater than about 500-fold less than that for mAChR M₃. In some embodiments, a compound of the invention can activate mAChR M₄ response with an EC₅₀ of about 5-fold less, about 10-fold less, about 20-fold less, about 30-fold less, about 50-fold less, about 100-fold less, about 200-fold less, about 300-fold less, about 400-fold less, or greater than about 500-fold less than that for mAChR M₅. In some embodiments, a compound of the invention can activate mAChR M₄ response with an EC₅₀ of 5-fold less, about 10-fold less, about 20-fold less, about 30-fold less than that for the M₂-M₅ receptors, of about 50-fold less, about 100-fold less, about 200-fold less, about 300-fold less, about 400-fold less, or greater than about 500-fold less than that for the mAChR M₁, M₂, M₃, or M₅ receptors.

The compounds of the invention may activate mAChR M₄ response in M₄-transfected CHO-K1 cells with an EC₅₀ of less than about 10 μM and exhibit a selectivity for the M₄ receptor vis-à-vis one or more of the mAChR M₁, M₂, M₃, or M₅ receptors. For example, in some embodiments, the compound can have an EC₅₀ of less than about 10 μM, of less than about 5 μM, of less than about 1 μM, of less than about 500 nM, of less than about 100 nM, or of less than about 50 nM; and the compound can also activate mAChR M₄ response with an EC₅₀ of about 5-fold less, 10-fold less, 20-fold less, 30-fold less, 50-fold less, 100-fold less, 200-fold less, 300-fold less, 400-fold less, or greater than about 500-fold less than that for mAChR M₁. In some embodiments, the compound can have an EC₅₀ of less than about 10 μM, of less than about 5 μM, of less than about 1 μM, of less than about 500 nM, of less than about 100 nM, or of less than about 50 nM; and the compound can also activate mAChR M₄ response with an EC₅₀ of about 5-fold less, about 10-fold less, about 20-fold less, about 30-fold less, about 50-fold less, about 100-fold less, about 200-fold less, about 300-fold less, about 400-fold less, or greater than about 500-fold less than that for mAChR M₂. In some embodiments, the compound can have an EC₅₀ of less than about 10 μM, of less than about 5 μM, of less than about 1 μM, of less than about 500 nM, of less than about 100 nM, or of less than about 50 nM; and the compound can also activate mAChR M₄ response with an EC₅₀ of about 5-fold less, about 10-fold less, about 20-fold less, about 30-fold less, about 50-fold less, about 100-fold less, about 200-fold less, about 300-fold less, about 400-fold less, or greater than about 500-fold less than that for mAChR M₃. In some embodiments, the compound can have an EC₅₀ of less than about 10 μM, of less than about 5 μM, of less than about 1 μM, of less than about 500 nM, of less than about 100 nM, or of less than about 50 nM; and the compound can also activate mAChR M₄ response with an EC₅₀ of about 5-fold less, about 10-fold less, about 20-fold less, about 30-fold less, about 50-fold less, about 100-fold less, about 200-fold less, about 300-fold less, about 400-fold less, or greater than about 500-fold less than that for mAChR M₅. In some embodiments, the compound can have an EC₅₀ of less than about 10 μM, of less than about 5 μM, of less than about 1 μM, of less than about 500 nM, of less than about 100 nM, or of less than about 50 nM; and the compound can also activate mAChR M₄ response with EC₅₀ of 5-fold less, about 10-fold less, about 20-fold less, about 30-fold less than that for the M₂-M₅ receptors, of about 50-fold less, about 100-fold less, about 200-fold less, about 300-fold less, about 400-fold less than that for mAChR M₂, M₃, or M₅ receptors, or greater than about 500-fold less than that for the mAChR M₁, M₂, M₃, or M₅ receptors.

In vivo efficacy for compounds of the invention may be measured in a number of preclinical rat behavioral models where known, clinically useful antipsychotics display similar positive responses. For example, compounds of the invention may reverse amphetamine-induced hyperlocomotion in male Sprague-Dawley rats at doses ranging from 1 to 100 mg/kg p.o.

Certain compound reference numbers in the experimentals below correspond to those used in Schemes I, II, III, IV, and V in the Detailed Description section and in the preparation of intermediates section.

c. Exemplified Compounds of the Invention

### Example 1. 5,6-dimethyl-4-[3-(trifluoromethyl)-7,8-dihydro-5H-1,6-naphthyridin-6-yl]thieno[2,3-d]pyrimidine (Compound 1)

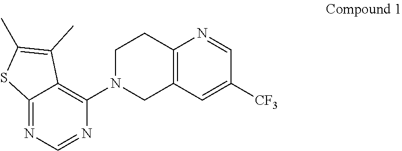 [SMILES: Cc1sc2ncnc(N3CCc4ncc(C(F)(F)F)cc4C3)c2c1C]

The preparation of compound 1 follows the reaction scheme below.

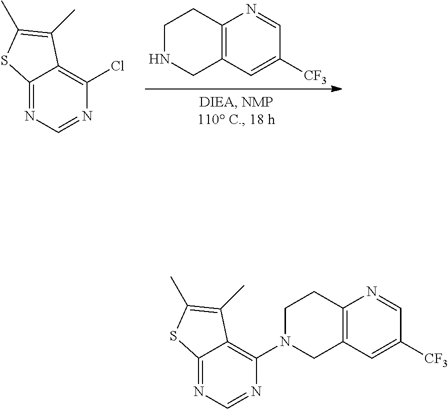 [SMILES: Cc1sc2ncnc(Cl)c2c1C ; FC(F)(F)c1cnc2c(c1)CNCC2 ; Cc1sc2ncnc(N3CCc4ncc(C(F)(F)F)cc4C3)c2c1C]

To a solution of 3-(trifluoromethyl)-5,6,7,8-tetrahydro-1,6-naphthyridine hydrochloride (12.8 mg) in NMP (0.5 mL) was added DIEA (0.04 mL, 0.2 mmol) followed by 4-chloro-5,6-dimethylthieno[2,3-d]pyrimidine (8.9 mg). The reaction mixture was stirred at 110° C. for 18 h. The reaction mixture was purified using RP-HPLC (10-60% MeCN in 0.1% TFA aqueous solution) to afford the title compound (7.5 mg). ¹H-NMR (400 MHz, CDCl₃) δ 8.77 (s, 1H), 8.59 (s, 1H), 7.77 (s, 1H), 4.76 (s, 2H), 3.88 (dd, J=6.0, 6.0 Hz, 2H), 3.35 (dd, J=6.2, 6.2 Hz, 2H), 2.52 (s, 3H), 2.50 (s, 3H); ES-MS [M+H]⁺=365.2.

### Example 2. 4-[3-(6,7-dihydro-5H-pyrazolo[1,5-a]pyrimidin-4-yl)-7,8-dihydro-5H-1,6-naphthyridin-6-yl]-6-methyl-thieno[2,3-d]pyrimidine (Compound 2)

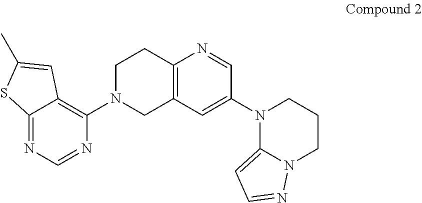 [SMILES: Cc1cc2c(N3CCc4ncc(N5CCCn6nccc65)cc4C3)ncnc2s1]

The preparation of compound 2 follows the reaction scheme below.

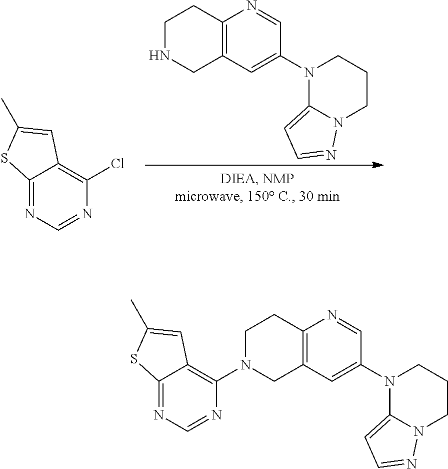 [SMILES: Cc1cc2c(Cl)ncnc2s1 ; c1cc2n(n1)CCCN2c1cnc2c(c1)CNCC2 ; Cc1cc2c(N3CCc4ncc(N5CCCn6nccc65)cc4C3)ncnc2s1]

3-(6,7-Dihydro-5H-pyrazolo[1,5-a]pyrimidin-4-yl)-5,6,7,8-tetrahydro-1,6-naphthyridine (17 mg), 4-chloro-6-methylthieno[2,3-d]pyrimidine (12 mg), DIEA (0.03 mL) and NMP (0.5 mL) were charged into a microwave vial. The mixture was subjected to microwave irradiation for 30 min at 150° C. The reaction mixture was purified using RP-HPLC (10-60% MeCN in 0.1% TFA aqueous solution) provided the title compound (7.9 mg). ¹H-NMR (400 MHz, DMSO-d₆) δ 8.41 (d, J=2.6 Hz, 1H), 8.39 (s, 1H), 7.69 (d, J=2.6 Hz, 1H), 7.47 (d, J=1.2 Hz, 1H), 5.65 (d, J=2.0 Hz, 1H), 5.02 (s, 2H), 4.15 (dd, J=5.9, 5.9 Hz, 2H), 4.11 (dd, J=6.2, 6.2 Hz, 2H), 3.69 (dd, J=6.2, 6.2 Hz, 2H), 3.05 (dd, J=5.8, 5.8 Hz, 2H), 2.58 (d, J=0.9 Hz, 3H), 2.24-2.18 (m, 2H); ES-MS [M+H]⁺=404.3.

### Example 3. 4-[3-(2-fluorophenoxy)-7,8-dihydro-5H-1,6-naphthyridin-6-yl]-6-methyl-thieno[2,3-d]pyrimidine (Compound 3)

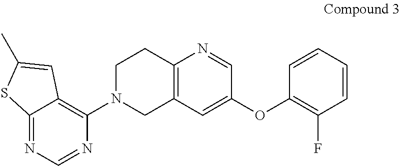 [SMILES: Cc1cc2c(N3CCc4ncc(Oc5ccccc5F)cc4C3)ncnc2s1]

Prepared in a similar manner as Compound 2 to provide the title compound. ¹H-NMR (400 MHz, DMSO-d₆) δ 8.38 (s, 1H), 8.23 (d, J=2.8 Hz, 1H), 7.46 (d, J=1.3 Hz, 1H), 7.45-7.40 (m, 2H), 7.29-7.22 (m, 3H), 5.00 (s, 2H), 4.15 (dd, J=5.9, 5.9 Hz, 2H), 3.07 (dd, J=5.8, 5.8 Hz, 2H), 2.56 (d, J=1.0, 3H); ES-MS [M+H]⁺=393.0.

### Example 4. 4-[3-(1-cyclopropylpyrazol-4-yl)-7,8-dihydro-5H-1,6-naphthyridin-6-yl]-7-methyl-thieno[3,2-d]pyrimidine (Compound 4)

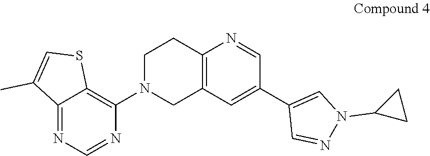 [SMILES: Cc1csc2c(N3CCc4ncc(-c5cnn(C6CC6)c5)cc4C3)ncnc12]

Prepared in a similar manner as Compound 2 to provide the title compound. ¹H-NMR (400 MHz, DMSO-d₆) δ 8.67 (d, J=2.1 Hz, 1H), 8.56 (s, 1H), 8.34 (s, 1H), 7.95 (d, J=0.5 Hz, 1H), 7.94 (d, J=2.0 Hz, 1H), 7.90 (d, J=1.2 Hz, 1H), 5.15 (s, 2H), 4.28 (t, J=2.1 Hz, 2H), 3.78-3.72 (m, 1H), 3.07 (t, J=5.9 Hz, 2H), 2.35 (d, J=1.0 Hz, 3H), 1.10-1.05 (m, 2H), 1.04-0.97 (m, 2H); ES-MS [M+H]⁺=389.0.

### Example 5. 7-methyl-4-[3-(2-methylthiazol-5-yl)-7,8-dihydro-5H-1,6-naphthyridin-6-yl]thieno[3,2-d]pyrimidine (Compound 5)

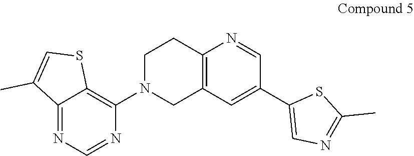

Prepared in a similar manner as Compound 2 to provide the title compound. ¹H-NMR (400 MHz, DMSO-d₆) δ 8.67 (d, J=2.2 Hz, 1H), 8.56 (s, 1H), 8.31 (s, 1H), 7.91 (d, J=0.5 Hz, 1H), 7.87 (d, J=2.0 Hz, 1H), 7.45 (d, J=1.1, 1H), 4.67 (s, 2H), 3.84 (t, J=6.0 Hz, 2H), 3.78-3.73 (m, 1H), 3.09 (t, J=5.8 Hz, 2H), 2.58 (d, J=1.0 Hz, 3H), 1.10-1.05 (m, 2H), 1.04-0.97 (m, 2H); ES-MS [M+H]⁺=389.0.

### Examples 6. 4-[3-(2-methylthiazol-5-yl)-7,8-dihydro-5H-1,6-naphthyridin-6-yl]thieno[2,3-d]pyrimidine (Compound 6)

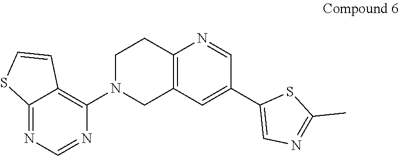 [SMILES: Cc1ncc(-c2cnc3c(c2)CN(c2ncnc4sccc24)CC3)s1]

Prepared in a similar manner as Compound 2 to provide the title compound. ¹H-NMR (400 MHz, DMSO-d₆) δ 8.69 (d, J=2.3 Hz, 1H), 8.47 (s, 1H), 8.12 (s, 1H), 8.04 (d, J=2.2 Hz, 1H), 7.80 (d, J=6.2 Hz, 1H) 7.70 (d, J=6.2 Hz, 1H), 5.13 (s, 2H), 4.22 (t, J=5.9 Hz, 2H), 3.13 (t, J=5.8 Hz, 2H), 2.70 (s, 3H); ES-MS [M+H]⁺=366.2.

The compounds shown in Table 1 were prepared in a similar manner as Compounds 1 to 6, with the appropriate starting materials.

TABLE 1

| Cpd. | | | |
|---|---|---|---|
| No. | Name | Structure | ES-MS [M + 1]⁺ |
| 7 | 2,5,6-trimethyl-4-[3- (trifluoromethyl)-7,8-dihydro- 5H-1,6-naphthyridin-6- yl]thieno[2,3-d]pyrimidine | 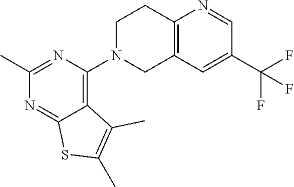 [SMILES: Cc1nc(N2CCc3ncc(C(F)(F)F)cc3C2)c2c(C)c(C)sc2n1] | 379.3 |
| 8 | 5-methyl-4-[3-(trifluoromethyl)- 7,8-dihydro-5H-1,6- naphthyridin-6-yl]thieno[2,3- d]pyrimidine | 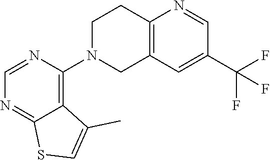 [SMILES: Cc1csc2ncnc(N3CCc4ncc(C(F)(F)F)cc4C3)c12] | 351.2 |
| 9 | 6-methyl-4-[3-(trifluoromethyl)- 7,8-dihydro-5H-1,6- naphthyridin-6-yl]thieno[2,3- d]pyrimidine | 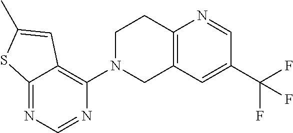 [SMILES: Cc1cc2c(N3CCc4ncc(C(F)(F)F)cc4C3)ncnc2s1] | 351.2 |
| 10 | 4-[3-(trifluoromethyl)-7,8- dihydro-5H-1,6-naphthyridin-6- yl]thieno[3,2-d]pyrimidine | 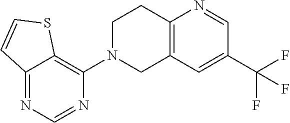 [SMILES: FC(F)(F)c1cnc2c(c1)CN(c1ncnc3ccsc13)CC2] | 337.2 |
| 11 | 7-methyl-4-[3-(trifluoromethyl)- 7,8-dihydro-5H-1,6- naphthyridin-6-yl]thieno[3,2- d]pyrimidine | 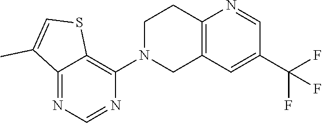 [SMILES: Cc1csc2c(N3CCc4ncc(C(F)(F)F)cc4C3)ncnc12] | 351.3 |
| 12 | 4-[3-(1,3-dimethylpyrazol-4-yl)- 7,8-dihydro-5H-1,6- naphthyridin-6-yl]-6-methyl- thieno[2,3-d]pyrimidine | 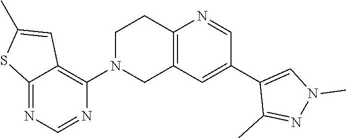 | 377.4 |
| 13 | 1-[6-(6-methylthieno[2,3- d]pyrimidin-4-yl)-7,8-dihydro- 5H-1,6-naphthyridin-3-yl]-2,3- dihydropyrido[2,3- b][1,4]oxazine | 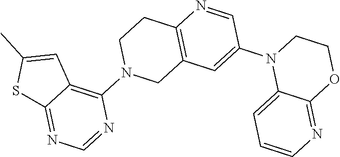 [SMILES: Cc1cc2c(N3CCc4ncc(N5CCOc6ncccc65)cc4C3)ncnc2s1] | 417.2 |
| 14 | 6-methyl-4-[3-(6-methyl-3- pyridyl)-7,8-dihydro-5H-1,6- naphthyridin-6-yl]thieno[2,3- d]pyrimidine | 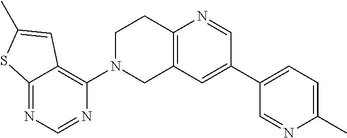 | 374.3 |
| 15 | 7-methyl-4-[3-(6-methyl-3-pyridyl)- 7,8-dihydro-5H-1,6-naphthyridin-6- yl]thieno[3,2-d]pyrimidine | 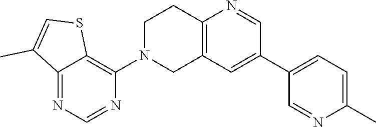 | 374 |
| 16 | 1-[6-(5-methylthieno[2,3- d]pyrimidin-4-yl)-7,8-dihydro-5H- 1,6-naphthyridin-3-yl]-2,3- dihydropyrido[2,3-b][1,4]oxazine | 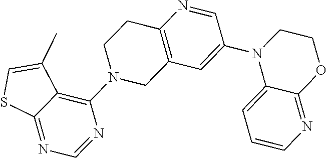 [SMILES: Cc1csc2ncnc(N3CCc4ncc(N5CCOc6ncccc65)cc4C3)c12] | 417.3 |
| 17 | 5-methyl-4-[3-(6-methyl-3-pyridyl)- 7,8-dihydro-5H-1,6-naphthyridin-6- yl]thieno[2,3-d]pyrimidine | 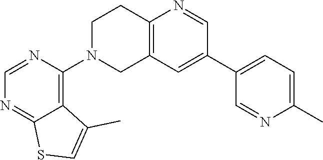 [SMILES: Cc1ccc(-c2cnc3c(c2)CN(c2ncnc4scc(C)c24)CC3)cn1] | 374.3 |
| 18 | 5-methyl-4-[3-(2-methylthiazol-5- yl)-7,8-dihydro-5H-1,6- naphthyridin-6-yl]thieno[2,3- d]pyrimidine | 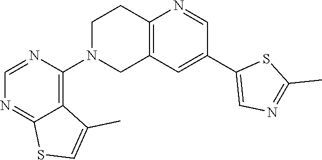 [SMILES: Cc1ncc(-c2cnc3c(c2)CN(c2ncnc4scc(C)c24)CC3)s1] | 380.3 |
| 19 | 4-[3-(2-fluoro-4-methyl-phenoxy)- 7,8-dihydro-5H-1,6-naphthyridin-6- yl]-5-methyl-thieno[2,3- d]pyrimidine | 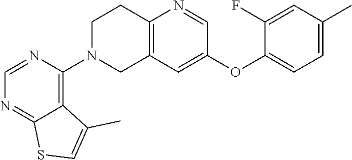 [SMILES: Cc1ccc(Oc2cnc3c(c2)CN(c2ncnc4scc(C)c24)CC3)c(F)c1] | 407 |
| 20 | 4-[3-(1-cyclopropylpyrazol-4-yl)- 7,8-dihydro-5H-1,6-naphthyridin-6- yl]-6-methyl-thieno[2,3- d]pyrimidine | 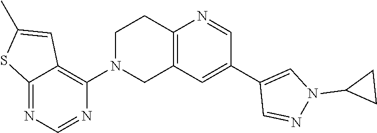 [SMILES: Cc1cc2c(N3CCc4ncc(-c5cnn(C6CC6)c5)cc4C3)ncnc2s1] | 389.3 |
| 21 | 6-methyl-4-[3-(2-methylthiazol-5- yl)-7,8-dihydro-5H-1,6- naphthyridin-6-yl]thieno[2,3- d]pyrimidine | 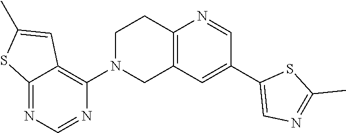 | 380.2 |
| 22 | 4-[3-(6,7-dihydro-5H-pyrazolo[1,5- a]pyrimidin-4-yl)-7,8-dihydro-5H- 1,6-naphthyridin-6-yl]-7-methyl- thieno[3,2-d]pyrimidine | 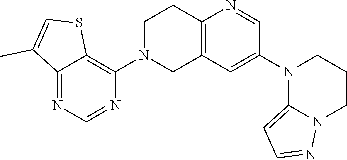 [SMILES: Cc1csc2c(N3CCc4ncc(N5CCCn6nccc65)cc4C3)ncnc12] | 404 |
| 23 | 4-[3-(1,3-dimethylpyrazol-4-yl)-7,8- dihydro-5H-1,6-naphthyridin-6-yl]- 7-methyl-thieno[3,2-d]pyrimidine | 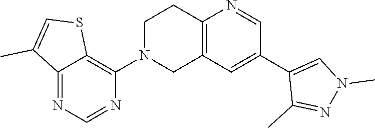 | 377 |
| 24 | 1-[6-(7-methylthieno[3,2- d]pyrimidin-4-yl)-7,8-dihydro-5H- 1,6-naphthyridin-3-yl]-2,3- dihydropyrido[2,3-b][1,4]oxazine | 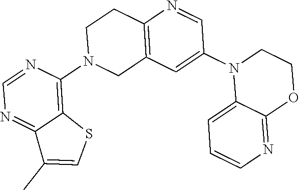 [SMILES: Cc1csc2c(N3CCc4ncc(N5CCOc6ncccc65)cc4C3)ncnc12] | 417 |
| 25 | 4-[3-(6,7-dihydro-5H-pyrazolo[1,5- a]pyrimidin-4-yl)-7,8-dihydro-5H- 1,6-naphthyridin-6-yl]-5-methyl- thieno[2,3-d]pyrimidine | 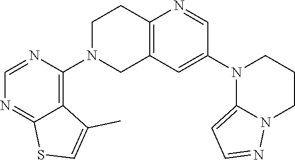 [SMILES: Cc1csc2ncnc(N3CCc4ncc(N5CCCn6nccc65)cc4C3)c12] | 404 |
| 26 | 4-[3-(1-cyclopropylpyrazol-4-yl)- 7,8-dihydro-5H-1,6-naphthyridin-6- yl]-5-methyl-thieno[2,3- d]pyrimidine | 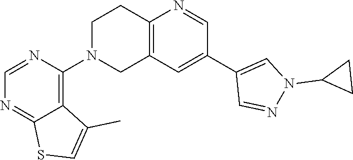 [SMILES: Cc1csc2ncnc(N3CCc4ncc(-c5cnn(C6CC6)c5)cc4C3)c12] | 389 |
| 27 | 4-[3-(2-fluoro-5-methyl-phenoxy)- 7,8-dihydro-5H-1,6-naphthyridin-6- yl]thieno[3,2-d]pyrimidine | 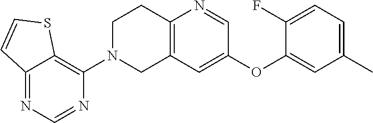 [SMILES: Cc1ccc(F)c(Oc2cnc3c(c2)CN(c2ncnc4ccsc24)CC3)c1] | 393 |
| 28 | 4-[3-(1,3-dimethylpyrazol-4-yl)-7,8- dihydro-5H-1,6-naphthyridin-6-yl]- 5-methyl-thieno[2,3-d]pyrimidine | 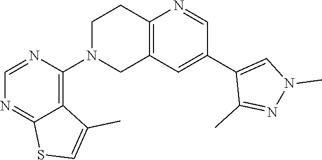 [SMILES: Cc1nn(C)cc1-c1cnc2c(c1)CN(c1ncnc3scc(C)c13)CC2] | 377.4 |
| 29 | 5-bromo-4-[3-(trifluoromethyl)-7,8- dihydro-5H-1,6-naphthyridin-6- yl]thieno[2,3-d]pyrimidine | 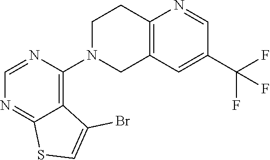 [SMILES: FC(F)(F)c1cnc2c(c1)CN(c1ncnc3scc(Br)c13)CC2] | 417.2 |
| 30 | 6-bromo-4-[3-(trifluoromethyl)-7,8- dihydro-5H-1,6-naphthyridin-6- yl]thieno[2,3-d]pyrimidine | 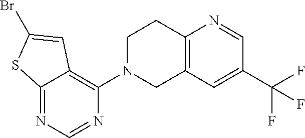 [SMILES: FC(F)(F)c1cnc2c(c1)CN(c1ncnc3sc(Br)cc13)CC2] | 416.8 |
| 31 | 4-[3-(6,7-dihydro-5H-pyrazolo[1,5- a]pyrimidin-4-yl)-7,8-dihydro-5H- 1,6-naphthyridin-6-yl]-6-isopropyl- thieno[2,3-d]pyrimidine | 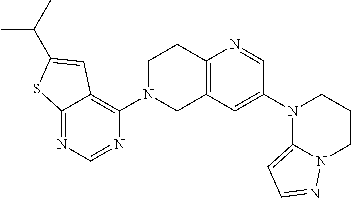 [SMILES: CC(C)c1cc2c(N3CCc4ncc(N5CCCn6nccc65)cc4C3)ncnc2s1] | 432.4 |
| 32 | 4-[3-(2-fluorophenoxy)-7,8- dihydro-5H-1,6-naphthyridin-6-yl]- 5-methyl-thieno[2,3-d]pyrimidine | 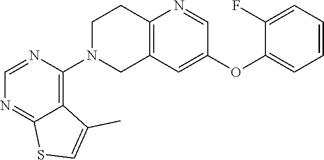 [SMILES: Cc1csc2ncnc(N3CCc4ncc(Oc5ccccc5F)cc4C3)c12] | 393.2 |
| 33 | 4-[3-(2-fluorophenoxy)-7,8- dihydro-5H-1,6-naphthyridin-6- yl]thieno[2,3-d]pyrimidine | 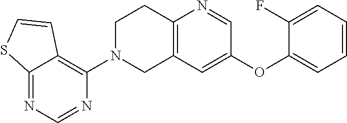 [SMILES: Fc1ccccc1Oc1cnc2c(c1)CN(c1ncnc3sccc13)CC2] | 379.2 |
| 34 | N-(2-methylpyrazol-3-yl)-6- thieno[2,3-d]pyrimidin-4-yl-7,8- dihydro-5H-1,6-naphthyridin-3- amine | 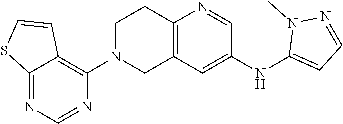 [SMILES: Cn1nccc1Nc1cnc2c(c1)CN(c1ncnc3sccc13)CC2] | 364.3 |
| 35 | 4-[6-(6-isopropylthieno[2,3- d]pyrimidin-4-yl)-7,8-dihydro-5H- 1,6-naphthyridin-3-yl]-3,5- dimethyl-isoxazole | 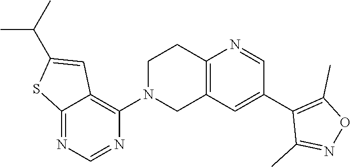 [SMILES: Cc1noc(C)c1-c1cnc2c(c1)CN(c1ncnc3sc(C(C)C)cc13)CC2] | 406.3 |
| 36 | 5-deuterio-4-[3-(trifluoromethyl)- 7,8-dihydro-5H-1,6-naphthyridin-6- yl]thieno[2,3-d]pyrimidine | 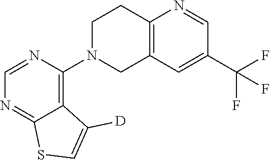 [SMILES: [2H]c1csc2ncnc(N3CCc4ncc(C(F)(F)F)cc4C3)c12] | 338.3 |
| 37 | 5-cyclopropyl-4-[3- (trifluoromethyl)-7,8-dihydro-5H- 1,6-naphthyridin-6-yl]thieno[2,3- d]pyrimidine | 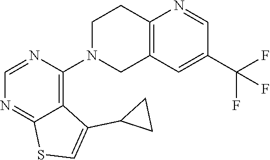 [SMILES: FC(F)(F)c1cnc2c(c1)CN(c1ncnc3scc(C4CC4)c13)CC2] | 377.2 |

### Example 7. 4-[3-(1-cyclopropylpyrazol-4-yl)-7,8-dihydro-5H-1,6-naphthyridin-6-yl]-5-methyl-furo[2,3-d]pyrimidine (Compound 38)

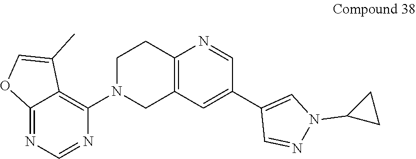 [SMILES: Cc1coc2ncnc(N3CCc4ncc(-c5cnn(C6CC6)c5)cc4C3)c12]

Prepared in a similar manner as Compound 2 to provide the title compound. ¹H-NMR (400 MHz, CDCl₃) δ 8.59 (d, J=1.9 Hz, 1H), 8.47 (s, 1H), 7.76 (s, 1H), 7.61 (s, 1H). 7.39 (d, J=1.3 Hz, 1H), 7.87 (d, J=2.0 Hz, 1H), 4.34 (s, 2H), 4.01 (dd, J=5.9, 5.9 Hz, 2H), 3.68-3.63 (m, 1H), 3.29 (dd, J=5.6, 5.6 Hz, 2H), 2.58 (d, J=1.0 Hz, 3H), 1.21-1.17 (m, 2H), 1.10-1.05 (m, 2H); ES-MS [M+H]⁺=373.3.

### Example 8. N-(2-fluoro-3-pyridyl)-6-(5-methylfuro[2,3-d]pyrimidin-4-yl)-7,8-dihydro-5H-1,6-naphthyridin-3-amine (Compound 39)

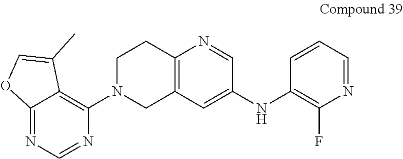 [SMILES: Cc1coc2ncnc(N3CCc4ncc(Nc5cccnc5F)cc4C3)c12]

Prepared in a similar manner as Compound 2 to provide the title compound. ¹H-NMR (400 MHz, DMSO-d₆) δ 8.37 (s, 1H), 8.31 (s, 1H), 8.24 (d, J=2.6 Hz, 1H), 7.76 (d, J=1.3 Hz, 1H), 7.72-7.67 (m, 2H), 7.45 (d, J=2.5 Hz, 1H), 7.21 (dddd, J=7.7, 4.6, 1.0, 1.0, 1H), 4.78 (s, 2H), 3.98 (dd, J=5.9, 5.9 Hz, 2H), 3.05 (dd, J=5.8, 5.8 Hz, 2H), 2.39 (d, J=1.3 Hz, 3H); ES-MS [M+H]⁺=377.4.

### Example 9. 3-methyl-4-[3-(trifluoromethyl)-7,8-dihydro-5H-1,6-naphthyridin-6-yl]isoxazolo[5,4-d]pyrimidine (Compound 40)

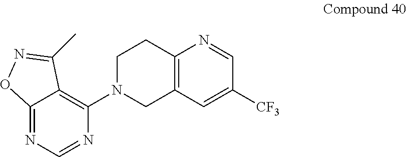 [SMILES: Cc1noc2ncnc(N3CCc4ncc(C(F)(F)F)cc4C3)c12]

Prepared in a similar manner as Compound 2 to provide the title compound. ¹H-NMR (400 MHz, CDCl₃) δ 8.76 (s, 1H), 8.55 (s, 1H), 7.78 (s, 1H), 5.03 (s, 2H), 4.18 (dd, J=6.0 Hz, 2H), 3.34 (dd, J=5.8, 5.8 Hz, 2H), 2.75 (s, 3H); ES-MS [M+H]⁺=336.3.

The compounds shown in Table 2 were prepared in a similar manner as Compounds 38 to 40, with the appropriate starting materials.

TABLE 2

| Cpd. | | | |
|---|---|---|---|
| No. | Name | Structure | ES-MS [M + 1]⁺ |
| 41 | 5-methyl-4-[3-(trifluoromethyl)- 7,8-dihydro-5H-1,6- naphthyridin-6-yl]furo[2,3- d]pyrimidine | 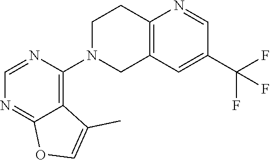 [SMILES: Cc1coc2ncnc(N3CCc4ncc(C(F)(F)F)cc4C3)c12] | 335.3 |
| 42 | 4-[3-(6,7-dihydro-5H- pyrazolo[1,5-a]pyrimidin-4-yl)- 7,8-dihydro-5H-1,6- naphthyridin-6-yl]-5-methyl- furo[2,3-d]pyrimidine | 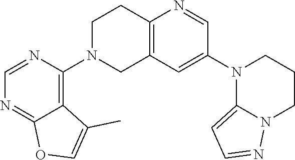 [SMILES: Cc1coc2ncnc(N3CCc4ncc(N5CCCn6nccc65)cc4C3)c12] | 388.4 |
| 43 | 4-[3-(1,3-dimethylpyrazol-4-yl)- 7,8-dihydro-5H-1,6- naphthyridin-6-yl]-3-methyl- isoxazolo[5,4-d]pyrimidine | 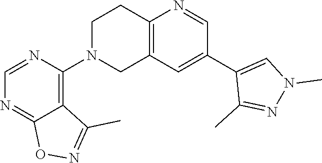 [SMILES: Cc1nn(C)cc1-c1cnc2c(c1)CN(c1ncnc3onc(C)c13)CC2] | 362.3 |
| 44 | 4-[3-(1,3-dimethylpyrazol-4-yl)- 7,8-dihydro-5H-1,6- naphthyridin-6-yl]-5-methyl- furo[2,3-d]pyrimidine | 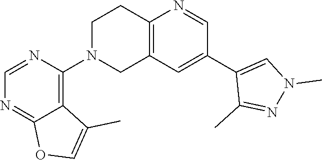 [SMILES: Cc1nn(C)cc1-c1cnc2c(c1)CN(c1ncnc3occ(C)c13)CC2] | 361.4 |
| 45 | 4-[3-(6,7-dihydro-5H- pyrazolo[1,5-a]pyrimidin-4-yl)- 7,8-dihydro-5H-1,6- naphthyridin-6-yl]-3-methyl- isoxazolo[5,4-d]pyrimidine | 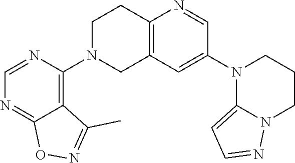 [SMILES: Cc1noc2ncnc(N3CCc4ncc(N5CCCn6nccc65)cc4C3)c12] | 389.4 |
| 46 | 4-[3-(1-cyclopropylpyrazol-4- yl)-7,8-dihydro-5H-1,6- naphthyridin-6-yl]-3-methyl- isoxazolo[5,4-d]pyrimidine | 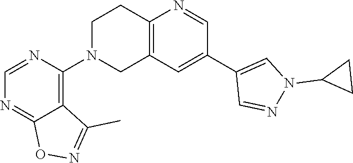 [SMILES: Cc1noc2ncnc(N3CCc4ncc(-c5cnn(C6CC6)c5)cc4C3)c12] | 374.3 |
| 47 | 1-[6-(5-methylfuro[2,3- d]pyrimidin-4-yl)-7,8-dihydro- 5H-1,6-naphthyridin-3-yl]-2,3- dihydropyrido[2,3- b][1,4]oxazine | 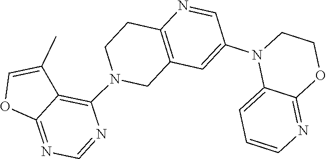 [SMILES: Cc1coc2ncnc(N3CCc4ncc(N5CCOc6ncccc65)cc4C3)c12] | 401.4 |
| 48 | 5-methyl-4-[3-(6-methyl-3- pyridyl)-7,8-dihydro-5H-1,6- naphthyridin-6-yl]furo[2,3- d]pyrimidine | 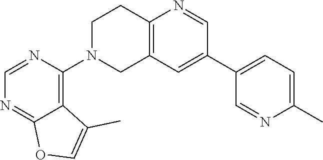 [SMILES: Cc1ccc(-c2cnc3c(c2)CN(c2ncnc4occ(C)c24)CC3)cn1] | 358.3 |
| 49 | 5-methyl-4-[3-(2-methylthiazol-5- yl)-7,8-dihydro-5H-1,6- naphthyridin-6-yl]furo[2,3- d]pyrimidine | 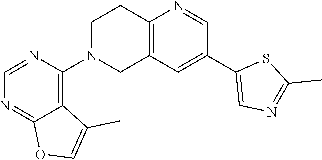 [SMILES: Cc1ncc(-c2cnc3c(c2)CN(c2ncnc4occ(C)c24)CC3)s1] | 364.4 |
| 50 | 1-[6-(3-methylisoxazolo[5,4- d]pyrimidin-4-yl)-7,8-dihydro-5H- 1,6-naphthyridin-3-yl]-2,3- dihydropyrido[2,3-b][1,4]oxazine | 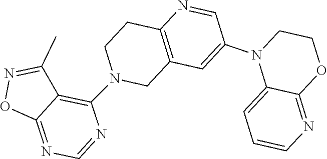 [SMILES: Cc1noc2ncnc(N3CCc4ncc(N5CCOc6ncccc65)cc4C3)c12] | 402.3 |
| 51 | 3-methyl-4-[3-(6-methyl-3-pyridyl)- 7,8-dihydro-5H-1,6-naphthyridin-6- yl]isoxazolo[5,4-d]pyrimidine | 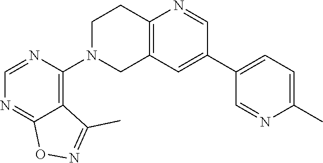 [SMILES: Cc1ccc(-c2cnc3c(c2)CN(c2ncnc4onc(C)c24)CC3)cn1] | 359.3 |
| 52 | 3-methyl-4-[3-(2-methylthiazol-5- yl)-7,8-dihydro-5H-1,6- naphthyridin-6-yl]isoxazolo[5,4- d]pyrimidine | 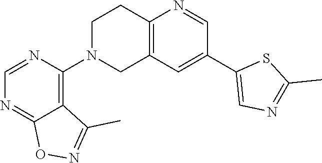 [SMILES: Cc1ncc(-c2cnc3c(c2)CN(c2ncnc4onc(C)c24)CC3)s1] | 365.3 |
| 53 | 4-[3-[(2-fluorophenyl)methyl]-7,8- dihydro-5H-1,6-naphthyridin-6-yl]- 5-methyl-furo[2,3-d]pyrimidine | 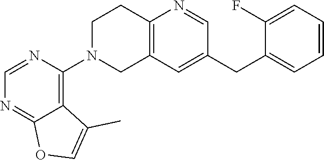 [SMILES: Cc1coc2ncnc(N3CCc4ncc(Cc5ccccc5F)cc4C3)c12] | 375 |
| 54 | 4-[3-[(3-fluorophenyl)methyl]-7,8- dihydro-5H-1,6-naphthyridin-6-yl]- 5-methyl-furo[2,3-d]pyrimidine | 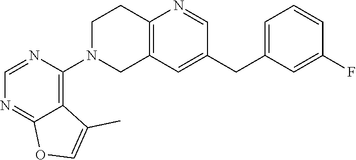 [SMILES: Cc1coc2ncnc(N3CCc4ncc(Cc5cccc(F)c5)cc4C3)c12] | 375 |
| 55 | 6-(5-methylfuro[2,3-d]pyrimidin- 4-yl)-N-(2-methylpyrazol-3-yl)- 7,8-dihydro-5H-1,6- naphthyridin-3-amine | 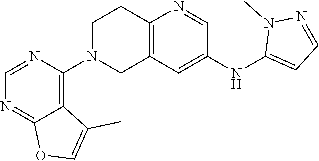 [SMILES: Cc1coc2ncnc(N3CCc4ncc(Nc5ccnn5C)cc4C3)c12] | 362.4 |
| 56 | 5-methyl-4-[3-(2-methylpyrazol- 3-yl)-7,8-dihydro-5H-1,6- naphthyridin-6-yl]furo[2,3- d]pyrimidine | 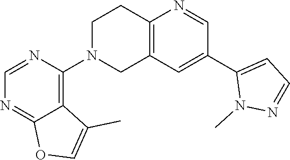 [SMILES: Cc1coc2ncnc(N3CCc4ncc(-c5ccnn5C)cc4C3)c12] | 347.4 |

### Example 10. 6-(2-chloro-6,7-dihydro-5H-cyclopenta[d]pyrimidin-4-yl)-3-(trifluoromethyl)-7,8-dihydro-5H-1,6-naphthyridine (Compound 57)

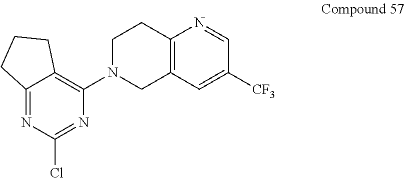 [SMILES: FC(F)(F)c1cnc2c(c1)CN(c1nc(Cl)nc3c1CCC3)CC2]

Prepared in a similar manner as Compound 2 at 70° C. to provide the title compound. ¹H-NMR (400 MHz, DMSO-d₆) δ 8.77 (d, J=1.2 Hz, 1H), 8.21 (d, J=1.5 Hz, 1H), 7.77 (s, 1H), 4.79 (s, 2H), 4.03 (dd, J=5.9, 5.9 Hz, 2H), 3.15-3.09 (m, 4H), 2.75 (dd, J=7.9, 7.9 Hz, 2H), 2.01 (q, J=15.4 Hz, 2H); ES-MS [M+H]⁺=355.2.

The compounds shown in Table 3 were prepared in a similar manner as Compound 57, with the appropriate starting materials.

TABLE 3

| Cpd. | | | |
|---|---|---|---|
| No. | Name | Structure | ES-MS [M + 1]⁺ |
| 58 | 6-(2-cyclopropyl-6,7-dihydro- 5H-cyclopenta[d]pyrimidin-4- yl)-3-(trifluoromethyl)-7,8- dihydro-5H-1,6-naphthyridine | 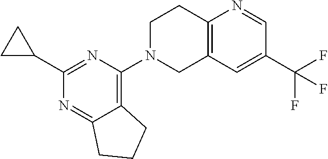 [SMILES: FC(F)(F)c1cnc2c(c1)CN(c1nc(C3CC3)nc3c1CCC3)CC2] | 361 |
| 59 | 6-(2-methyl-6,7-dihydro-5H- cyclopenta[d]pyrimidin-4-yl)-3- (trifluoromethyl)-7,8-dihydro- 5H-1,6-naphthyridine | 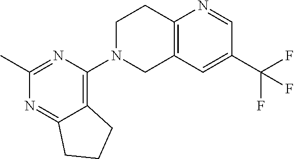 [SMILES: Cc1nc2c(c(N3CCc4ncc(C(F)(F)F)cc4C3)n1)CCC2] | 335 |

### Example 11: 6-(1-methylpyrazolo[4,3-d]pyrimidin-7-yl)-3-(trifluoromethyl)-7,8-dihydro-5H-1,6-naphthyridine (Compound 60)

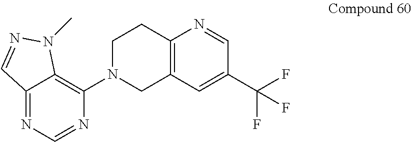 [SMILES: Cn1ncc2ncnc(N3CCc4ncc(C(F)(F)F)cc4C3)c21]

Prepared in a similar manner as Compound 2 to provide the title compound. ¹H-NMR (400 MHz, DMSO-d₆) δ 8.80 (d, J=1.1 Hz, 1H), 8.47 (s, 1H), 8.33 (s, 1H), 8.33 (s, 1H), 5.19 (s, 2H), 4.28 (t, J=5.9 Hz, 2H), 3.93 (s, 3H), 3.17 (t, J=5.2 Hz, 2H); ES-MS [M+H]⁺=335.0.

### Example 12: 6-(7-methylpyrrolo[2,3-d]pyrimidin-4-yl)-3-(trifluoromethyl)-7,8-dihydro-5H-1,6-naphthyridine (compound 61)

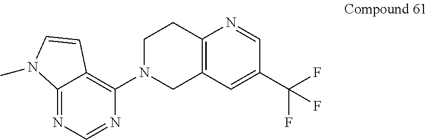 [SMILES: Cn1ccc2c(N3CCc4ncc(C(F)(F)F)cc4C3)ncnc21]

Prepared in a similar manner as Compound 2 to provide the title compound. ¹H-NMR (400 MHz, DMSO-d₆) δ 8.77 (d, J=1.0 Hz, 1H), 8.28 (d, J=1.4 Hz, 1H), 8.24 (s, 1H), 7.31 (d, J=3.6 Hz, 1H), 6.86 (d, J=3.6 Hz, 1H), 5.17 (s, 2H), 4.25 (t, J=4.3 Hz, 2H) 3.73 (s, 3H), 3.14 (t, J=5.8 Hz, 2H); ES-MS [M+H]⁺=334.0.

### Example 13: 2-chloro-4-[3-(trifluoromethyl)-7,8-dihydro-5H-1,6-naphthyridin-6-yl]-5,7-dihydrofuro[3,4-d]pyrimidine (Compound 62)

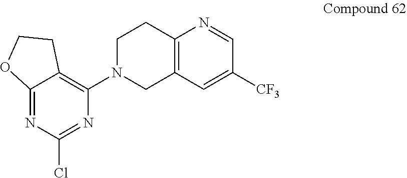 [SMILES: FC(F)(F)c1cnc2c(c1)CN(c1nc(Cl)nc3c1CCO3)CC2]

Prepared in a similar manner as Compound 2 at 70° C., using 2,4-dichloro-5,7-dihydrofuro[3,4-d]pyrimidine, to provide the title compound as a white powder. ¹H-NMR (400 MHz, DMSO-d₆) δ 8.78 (d, J=1.1 Hz, 1H), 8.24 (d, J=1.3 Hz, 1H), 5.34 (dd, J=2.6, 2.6 Hz, 2H), 4.92 (s, 2H), 4.80 (dd, J=2.6, 2.6 Hz, 2H), 3.90 (dd, J=5.7, 5.7 Hz, 2H), 3.11 (dd, J=5.7, 5.7 Hz, 2H); ES-MS [M+H]=357.3.

### Example 14: 1-[6-(2-chloro-5,7-dihydrofuro[3,4-d]pyrimidin-4-yl)-7,8-dihydro-5H-1,6-naphthyridin-3-yl]-2,3-dihydropyrido[2,3-b][1,4]oxazine (Compound 63)

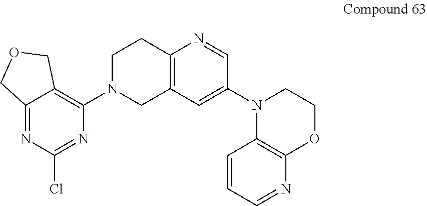 [SMILES: Clc1nc2c(c(N3CCc4ncc(N5CCOc6ncccc65)cc4C3)n1)COC2]

Prepared in a similar manner as Compound 2 at 70° C., using 2,4-dichloro-5,7-dihydrofuro[3,4-d]pyrimidine, to provide the title compound as a white powder. ¹H-NMR (400 MHz, DMSO-d₆) δ 8.38 (d, J=2.6 Hz, 1H), 7.71 (d, J=2.4 Hz, 1H), 7.61 (dd, J=6.3, 1.6 Hz, 1H), 7.12 (dd, J=7.9, 1.6 Hz, 1H), 6.80 (dd, J=7.9, 4.7 Hz, 1H), 5.33 (dd, J=2.6, 2.6 Hz, 2H), 4.82 (s, 2H), 4.80 (dd, J=2.6, 2.6 Hz, 2H), 4.41 (dd, J=4.2, 4.2 Hz, 2H), 3.89 (dd, J=5.6, 5.6 Hz, 2H), 3.72 (dd, J=4.3, 4.3 Hz, 2H), 3.00 (dd, J=5.9, 5.9 Hz, 2H); ES-MS [M+H]⁺=423.2.

The compounds shown in Table 4 were prepared in a similar manner as Compounds 62-63, with the appropriate starting materials.

TABLE 4

| Cpd. | | | |
|---|---|---|---|
| No. | Name | Structure | ES-MS [M + 1]⁺ |
| 64 | 2-chloro-4-(3-(6,7- dihydropyrazolo[1,5- a]pyrimidin-4(5H)-yl)-7,8- dihydro-1,6-naphthyridin-6(5H)- yl)-5,7-dihydrofuro[3,4- d]pyrimidine | 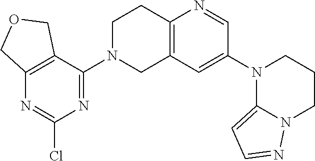 [SMILES: Clc1nc2c(c(N3CCc4ncc(N5CCCn6nccc65)cc4C3)n1)COC2] | 410 |
| 65 | (R)-2-chloro-4-(3-(2- methylmorpholino)-7,8-dihydro- 1,6-naphthyridin-6(5H)-yl)-5,7- dihydrofuro[3,4-d]pyrimidine | 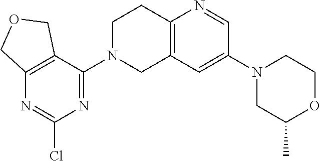 [SMILES: C[C@@H]1CN(c2cnc3c(c2)CN(c2nc(Cl)nc4c2COC4)CC3)CCO1] | 388 |
| 66 | 2-chloro-4-(3-cyclopropyl-7,8- dihydro-1,6-naphthyridin-6(5H)- yl)-5,7-dihydrofuro[3,4- d]pyrimidine | 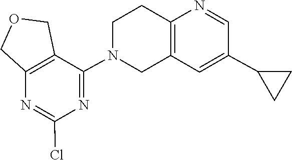 [SMILES: Clc1nc2c(c(N3CCc4ncc(C5CC5)cc4C3)n1)COC2] | 329 |
| 67 | 6-(2-chloro-5,7-dihydrofuro[3,4- d]pyrimidin-4-yl)-N-(1-methyl- 1H-pyrazol-5-yl)-5,6,7,8- tetrahydro-1,6-naphthyridin-3- amine | 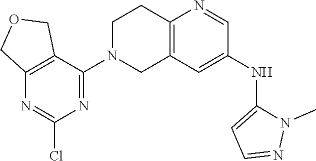 [SMILES: Cn1nccc1Nc1cnc2c(c1)CN(c1nc(Cl)nc3c1COC3)CC2] | 384 |
| 68 | 6-(2-chloro-5,7-dihydrofuro[3,4- d]pyrimidin-4-yl)-N-(2- fluoropyridin-3-yl)-5,6,7,8- tetrahydro-1,6-naphthyridin-3- amine | 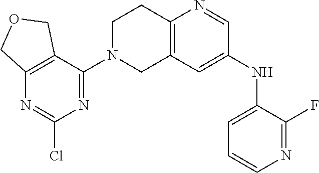 [SMILES: Fc1ncccc1Nc1cnc2c(c1)CN(c1nc(Cl)nc3c1COC3)CC2] | 399 |
| 69 | 2-chloro-4-(3-(2-fluorophenoxy)- 7,8-dihydro-1,6-naphthyridin- 6(5H)-yl)-5,7-dihydrofuro[3,4- d]pyrimidine | 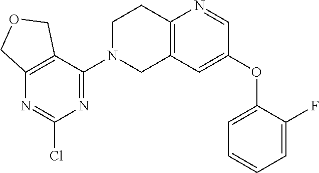 [SMILES: Fc1ccccc1Oc1cnc2c(c1)CN(c1nc(Cl)nc3c1COC3)CC2] | 399 |

d. Biological Activity of the Compounds of the Invention

### Example 14. Biological Activity

### A. Cell Lines Expressing Muscarinic Acetylcholine Receptors

Human M₄ cDNA, along with the chimeric G protein G_{qi5}, were transfected into Chinese hamster ovary (CHO-K1) cells purchased from the American Type Culture Collection using commercially available Lipofectamine 2000 transfection reagent (Dalby, B. et al., Methods (2004) 33(2), 95-103). hM₄-G_{qi5} cells were grown in Ham's F-12 medium containing 10% heat-inactivated fetal bovine serum (FBS), 20 mM HEPES, 50 μg/mL G418 sulfate (CAS #108321-42-2), and 500 μg/mL Hygromycin B. rM₄-G_{qi5} cells were grown in DMEM containing 10% heat-inactivated FBS, 20 mM HEPES, 400 μg/mL G418 sulfate, and 500 μg/mL Hygromycin B.

### B. Cell-Based Functional Assay of Muscarinic Acetylcholine Receptor Activity

For high throughput measurement of agonist-evoked increases in intracellular calcium, CHO-K1 cells stably expressing muscarinic receptors were plated in growth medium lacking G418 sulfate and hygromycin at 15,000 cells/20 μL/well in Greiner 384-well black-walled, tissue culture (TC)-treated, clear-bottom plates (VWR). Cells were incubated overnight at 37° C. and 5% CO₂. The next day, cells were washed using an ELX 405 (BioTek) with assay buffer; the final volume was then aspirated to 20 μL. Next, 20 μL of a 2.3 μM stock of Fluo-4/acetoxymethyl ester (Invitrogen, Carlsbad, CA), prepared as a 2.3 mM stock in DMSO and mixed in a 1:1 ratio with 10% (w/v) Pluronic F-127 and diluted in assay buffer, was added to the wells and the cell plates were incubated for 50 minutes at 37° C. and 5% CO₂. Dye was removed by washing with the ELX 405 and the final volume was aspirated to 20 μL. Compound master plates were formatted in an 11 point concentration-response curve (CRC) format (1:3 dilutions) in 100% DMSO with a starting concentration of 10 mM using a BRAVO liquid handler (Agilent). Test compound CRCs were then transferred to daughter plates (240 nL) using the Echo acoustic plate reformatter (Labcyte, Sunnyvale, CA) and then diluted into assay buffer (40 μL) to a 2× stock using a Thermo Fisher Combi (Thermo Fisher Scientific, Waltham, MA).

Calcium flux was measured using the Functional Drug Screening System (FDSS) 6000 or 7000 (Hamamatsu Corporation, Tokyo, Japan) as an increase in the fluorescent static ratio. Compounds were applied to cells (20 μL, 2×) using the automated system of the FDSS at 2-4 seconds into the protocol and the data were collected at 1 Hz. At 144 seconds, 10 μL of an EC₂₀ concentration of the muscarinic receptor agonist acetylcholine was added (5×), followed by the addition of 12 μL of an EC₅₀ concentration of acetylcholine at the 230 second time point (5×). Agonist activity was analyzed as a concentration-dependent increase in calcium mobilization upon compound addition. Positive allosteric modulator activity was analyzed as a concentration-dependent increase in the EC₂₀ acetylcholine response. With the acetylcholine EC₂₀ set as baseline, the test compound EC₅₀ is determined as the concentration that produces an increase above baseline of 50% of the maximum increase in acetylcholine response elicted by the test compound. Antagonist activity was analyzed as a concentration-dependent decrease in the EC₈₀ acetylcholine response. Concentration-response curves were generated using a four-parameter logistical equation in XLFit curve fitting software (IDBS, Bridgewater, NJ) for Excel (Microsoft, Redmond, WA) or Prism (GraphPad Software, Inc., San Diego, CA).

The above-described assay was also operated in a second mode where an appropriate fixed concentration of the present compounds was added to the cells after establishment of a fluorescence baseline for about 3 seconds, and the response in cells was measured. 140 seconds later, the appropriate concentration of agonist was added and the calcium response (maximum-local minima response) was measured. The EC₅₀ values for the agonist in the presence of test compound were determined by nonlinear curve fitting. A decrease in the EC₅₀ value of the agonist with increasing concentrations of the present compounds (a leftward shift of the agonist concentration-response curve) is an indication of the degree of muscarinic positive allosteric modulation at a given concentration of the present compound. An increase in the EC₅₀ value of the agonist with increasing concentrations of the present compounds (a rightward shift of the agonist concentration response curve) is an indication of the degree of muscarinic antagonism at a given concentration of the present compound. The second mode also indicates whether the present compounds also affect the maximum response of the muscarinic receptor to agonists.

C. Activity of Compounds in a mAChR M₄ Cell-Based Assay

Compounds were synthesized as described above. Activity (EC₅₀ and Eₘₐₓ) was determined in the mAChR M₄ cell-based functional assay as described above and the data are shown in Table 5. The compound numbers correspond to the compound numbers used in Examples 1-13 and Tables 1, 2, and 3.

TABLE 5
Compound Data

| | Human | |
|---|---|---|
| | M₄ EC₅₀ | Eₘₐₓ |
| No. | (μM) | (%)* |
| 1 | 0.61 | 85 |
| 2 | 0.03 | 106 |
| 3 | 0.63 | 80 |
| 4 | 0.77 | 43 |
| 5 | 0.31 | 35 |
| 6 | 0.10 | 45 |
| 7 | 3.28 | 88 |
| 8 | 0.03 | 78 |
| 9 | 0.03 | 76 |
| 10 | 0.12 | 79 |
| 11 | 0.50 | 63 |
| 12 | 0.23 | 74 |
| 13 | 0.07 | 121 |
| 14 | 0.19 | 97 |
| 15 | 0.55 | 45 |
| 16 | 0.06 | 91 |
| 17 | 0.18 | 79 |
| 18 | 0.47 | 72 |
| 19 | 1.84 | 58 |
| 20 | 0.47 | 91 |
| 21 | 0.25 | 88 |
| 22 | 0.28 | 85 |
| 23 | 0.56 | 59 |
| 24 | 0.22 | 70 |
| 25 | 0.06 | 96 |
| 26 | 0.28 | 75 |
| 27 | 2.38 | 36 |
| 28 | 0.09 | 78 |
| 29 | 0.31 | 74 |
| 30 | 0.08 | 88 |
| 31 | 0.44 | 58 |
| 32 | 0.56 | 59 |
| 33 | 0.20 | 46 |
| 34 | 0.10 | 55 |
| 35 | 1.23 | 22 |
| 36 | 0.16 | 78 |
| 37 | 1.39 | 22 |
| 38 | 0.06 | 83 |
| 39 | 0.08 | 94 |
| 40 | 0.71 | 94 |
| 41 | 0.05 | 86 |
| 42 | 0.01 | 99 |
| 43 | 0.59 | 80 |
| 44 | 0.12 | 95 |
| 45 | 0.09 | 93 |
| 46 | 0.37 | 75 |
| 47 | 0.05 | 125 |
| 48 | 0.09 | 102 |
| 49 | 0.13 | 68 |
| 50 | 0.24 | 100 |
| 51 | 0.51 | 89 |
| 52 | 0.55 | 92 |
| 53 | 0.28 | 81 |
| 54 | 0.50 | 78 |
| 55 | 0.06 | 94 |
| 56 | 0.08 | 74 |
| 57 | 0.10 | 69 |
| 58 | 1.48 | 88 |
| 59 | 1.03 | 87 |
| 60 | 6.06 | 31 |
| 61 | 0.83 | 80 |
| 62 | 3.29 | 58 |
| 63 | 0.37 | 56 |
| 64 | 0.30 | 62 |
| 65 | 3.26 | 35 |
| 66 | 0.58 | 54 |
| 67 | 1.22 | 51 |
| 68 | 0.92 | 51 |
| 69 | 0.99 | 42 |

*% ACh maximum at 30 μM.

What is claimed is:

1. A compound of formula (I), or a pharmaceutically acceptable salt thereof, wherein: 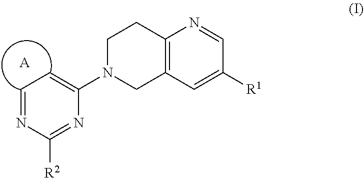
A is selected from a 5-membered heteroarene, a 5-membered heterocycle, and a 5-membered alicyclic ring system, wherein the heteroarene and heterocycle contain 1 to 2 heteroatoms independently selected from the group consisting of N, O, and S;
wherein the 5-membered heteroarene, the 5-membered heterocycle, and the 5-membered alicyclic ring system of A are unsubstituted or substituted with 1, 2, or 3 substituents independently selected from C₁₋₃ alkyl, C₁₋₄ fluoroalkyl, C₃₋₆ cycloalkyl, and halogen;
R¹ is selected from C₁₋₄ fluoroalkyl, C₃₋₆ cycloalkyl, phenyl, 5- or 6-membered heteroaryl, —NR^{a}R^{b} and -LR^{c};
L is CH₂ or O;
wherein R^{a} is a 5- or 6-membered heteroaryl or phenyl;
wherein R^{b} is hydrogen; or
wherein R^{a} and R^{b} together with the N to which they are attached form a heterocyclyl;
wherein R^{c} is 5- or 6-membered heteroaryl or phenyl;
wherein each phenyl, 5- or 6-membered heteroaryl, cycloalkyl, and heterocyclyl of R¹, R^{a}, or R^{c} is unsubstituted or substituted with 1, 2, or 3 substituents independently selected from C₁₋₄ alkyl, C₁₋₄ fluoroalkyl, —NR^{d}R^{e}, halogen, and C₃₋₆ cycloalkyl;
wherein R^{d} and R^{e} are independently selected from hydrogen and C₁₋₄ alkyl;
R² is selected from hydrogen, C₁₋₄ alkyl, halogen, —NR^{g}R^{h}, —ORⁱ and C₃₋₆ cycloalkyl;
wherein R^{g} is independently selected from C₁₋₄ alkyl, C₁₋₃ hydroxyalkyl, C₃₋₆ cycloalkyl, phenyl, and 5- or 6-membered heteroaryl;
wherein R^{h} is independently selected from hydrogen and C₁₋₄ alkyl; or
wherein R^{g} and R^{h} together with the N to which they are attached form a heterocyclyl;
wherein Rⁱ is C₁₋₄ alkyl;
wherein each 5- or 6-membered heteroaryl, heterocyclyl, phenyl, and C₃₋₆ cycloalkyl of R² is unsubstituted or substituted with 1, 2, or 3 substituents independently selected from —NR^{j}R^{k}, C₁₋₄ fluoroalkyl, hydroxyl, C₁₋₄ alkyl, halogen, C₃₋₆ cycloalkyl; and
wherein Rⁱ and R^{k} are independently selected from hydrogen and C₁₋₄ alkyl.

2. A compound of claim 1, or a pharmaceutically acceptable salt thereof, wherein:
A is selected from a 5-membered heteroarene containing 1 to 2 heteroatoms independently selected from the group consisting of N, O, and S, and a 5-membered alicyclic ring system;
wherein the 5-membered heteroarene, and the 5-membered alicyclic ring system of A are unsubstituted or substituted with 1 or 2 substituents independently selected from C₁₋₃ alkyl, C₁₋₄ fluoroalkyl, C₃₋₆ cycloalkyl, and halogen;
R¹ is selected from C₁₋₄ fluoroalkyl, C₃₋₆ cycloalkyl, 5- or 6-membered heteroaryl, —CH₂—R^{c}, —NR^{a}R^{b} and —OR^{c};
wherein R^{a} is a 5- or 6-membered heteroaryl;
wherein R^{b} is hydrogen; or
wherein R^{a} and R^{b} together with the N to which they are attached form a heterocyclyl;
wherein R^{c} is phenyl;
wherein each phenyl, 5- or 6-membered heteroaryl, and heterocyclyl of R¹, R^{a}, or R^{c} are unsubstituted or substituted with 1, 2, or 3 substituents independently selected from C₁₋₃ alkyl, C₁₋₄ fluoroalkyl, —NR^{d}R^{e}, halogen, and C₃₋₆ cycloalkyl;
wherein R^{d} and R^{e} are independently selected from hydrogen and C₁₋₃ alkyl;
R² is selected from hydrogen, C₁₋₃ alkyl, halogen, —NR^{g}R^{h}, —ORⁱ and C₃₋₆ cycloalkyl;
wherein R^{g} is independently selected from C₁₋₃ alkyl, C₁₋₃ hydroxyalkyl, C₃₋₆ cycloalkyl, phenyl, and 5- or 6-membered heteroaryl;
wherein R^{h} is independently selected from hydrogen and C₁₋₃ alkyl; or
wherein R^{g} and R^{h} together with the N to which they are attached form a heterocyclyl;
wherein Rⁱ is C₁₋₃ alkyl;
wherein each 5- or 6-membered heteroaryl, heterocyclyl, phenyl, and C₃₋₆ cycloalkyl of R² are unsubstituted or substituted with 1, 2, or 3 substituents independently selected from —NR^{j}R^{k}, C₁₋₄ fluoroalkyl, hydroxyl, C₁₋₃ alkyl, halogen, C₃₋₆ cycloalkyl; and
wherein Rⁱ and R^{k} are independently selected from hydrogen and C₁₋₃ alkyl.

3. A compound according to claim 1 or 2, or a pharmaceutically acceptable salt thereof, wherein the 5-membered heteroarene of A is selected from thieno, furo, isoxazolo, pyrazolo, and pyrrolo, each being unsubstituted or substituted with 1 or 2 substituents independently selected from C₁₋₃ alkyl, C₃₋₆ cycloalkyl, and halogen.

4. A compound according to any of claims 1-3, or a pharmaceutically acceptable salt thereof, wherein the 5-membered alicyclic ring system of A is an unsubstituted 5-membered alicyclic ring system.

5. A compound according to any of claims 1-3, or a pharmaceutically acceptable salt thereof, wherein the 5-membered heterocycle of A contains one heteroatom selected from oxygen.

6. A compound according to any of claims 1-5, or a pharmaceutically acceptable salt thereof, wherein A is selected from thieno unsubstituted or substituted with 1 or 2 substituents independently selected from C₁₋₃ alkyl, C₃₋₆ cycloalkyl, and halogen; furo unsubstituted or substituted with 1 methyl; isoxazolo unsubstituted or substituted with 1 methyl; pyrazolo unsubstituted or substituted with 1 methyl; and pyrrolo unsubstituted or substituted with 1 methyl.

7. A compound according to any of claims 1-6 having the formula (Ia), or a pharmaceutically acceptable salt thereof, wherein: 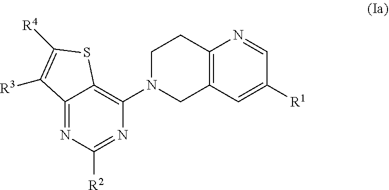
R³ is selected from hydrogen and C₁₋₃ alkyl; and
R⁴ is selected from hydrogen, halogen and C₁₋₃ alkyl.

8. A compound according to claim 7, or a pharmaceutically acceptable salt thereof, wherein R³ is methyl.

9. A compound according to claim 7, or a pharmaceutically acceptable salt thereof, wherein R³ is hydrogen.

10. A compound according to any of claims 7-9, or a pharmaceutically acceptable salt thereof, wherein R⁴ is methyl.

11. A compound according to any of claims 7-9, or a pharmaceutically acceptable salt thereof, wherein R⁴ is hydrogen.

12. A compound according to any of claims 7-9, or a pharmaceutically acceptable salt thereof, wherein R⁴ is bromo.

13. A compound according to any of claims 1-6 having the formula (Ib), or a pharmaceutically acceptable salt thereof, wherein: 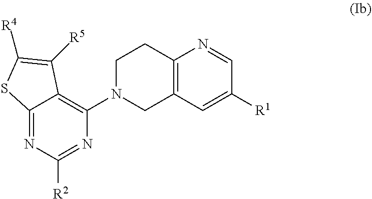
R⁴ is selected from hydrogen, halogen, and C₁₋₃ alkyl; and
R⁵ is selected from hydrogen, halogen, C₃₋₆ cycloalkyl and C₁₋₃ alkyl.

14. A compound according to claim 13, or a pharmaceutically acceptable salt thereof, wherein R⁴ is hydrogen.

15. A compound according to claim 13, or a pharmaceutically acceptable salt thereof, wherein R⁴ is bromo.

16. A compound according to claim 13, or a pharmaceutically acceptable salt thereof, wherein R⁴ is methyl.

17. A compound according to any of claims 13-16, or a pharmaceutically acceptable salt thereof, wherein R⁵ is hydrogen.

18. A compound according to any of claims 13-16, or a pharmaceutically acceptable salt thereof, wherein R⁵ is bromo.

19. A compound according to any of claims 13-16, or a pharmaceutically acceptable salt thereof, wherein R⁵ is cyclopropyl.

20. A compound according to any of claims 13-16, or a pharmaceutically acceptable salt thereof, wherein R⁵ is methyl.

21. A compound according to any of claims 1-6 having the formula (Ic), or a pharmaceutically acceptable salt thereof, wherein: 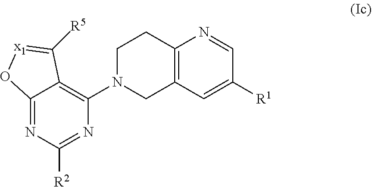
X₁ is selected from N and CR⁶,
wherein R⁶ is selected from hydrogen and C₁₋₃ alkyl; and
R⁵ is selected from hydrogen and C₁₋₃ alkyl.

22. A compound according to claim 21, or a pharmaceutically acceptable salt thereof, wherein X₁ is N.

23. A compound according to claim 21, or a pharmaceutically acceptable salt thereof, wherein X₁ is CR⁶.

24. A compound according to claim 23, or a pharmaceutically acceptable salt thereof, wherein X₁ is CH.

25. A compound according to claim 21 to 24, or a pharmaceutically acceptable salt thereof, wherein R⁵ is methyl.

26. A compound according to any of claims 1-5 having the formula (Id), or a pharmaceutically acceptable salt thereof, wherein: 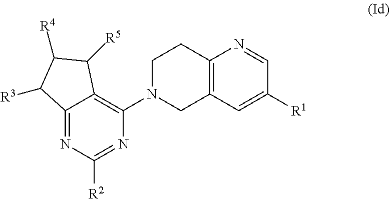
R³ is selected from hydrogen and C₁₋₃ alkyl;
R⁴ is selected from hydrogen and C₁₋₃ alkyl; and
R⁵ is selected from hydrogen and C₁₋₃ alkyl.

27. A compound according to claim 26, or a pharmaceutically acceptable salt thereof, wherein R³, R⁴, R⁵ are hydrogen.

28. A compound according to any of claims 1-6 having the formula (Ie), or a pharmaceutically acceptable salt thereof, wherein: 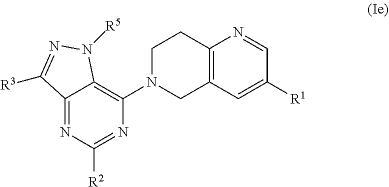
R³ is selected from hydrogen and C₁₋₃ alkyl; and
R⁵ is selected from hydrogen and C₁₋₃ alkyl.

29. A compound according to claim 28, or a pharmaceutically acceptable salt thereof, wherein R³ is hydrogen and R⁵ is methyl.

30. A compound according to any of claims 1-6 having the formula (If), or a pharmaceutically acceptable salt thereof, wherein: 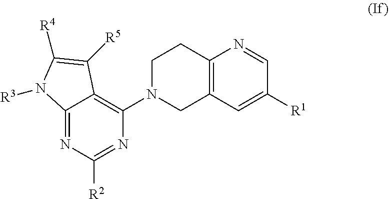
R³ is selected from hydrogen and C₁₋₃ alkyl;
R⁴ is selected from hydrogen and C₁₋₃ alkyl; and
R⁵ is selected from hydrogen and C₁₋₃ alkyl.

31. A compound according to claim 30, or a pharmaceutically acceptable salt thereof, wherein R³ is methyl; and R⁴ and R⁵ are hydrogen.

32. A compound according to any of claims 1-5 having the formula (Ig), or a pharmaceutically acceptable salt thereof

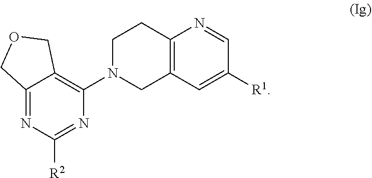

33. A compound according to any of claims 1 to 32, or a pharmaceutically acceptable salt thereof, wherein the C₁₋₄fluoroalkyl of R¹ is trifluoromethyl.

34. A compound according to any of claims 1 to 33, or a pharmaceutically acceptable salt thereof, wherein the 5- or 6-membered heteroaryl of R¹ is selected from isoxazolyl, pyridyl, thiazolyl, and pyrazolyl, each are unsubstituted or substituted with 1 or 2 substituents independently selected from a C₁₋₃ alkyl, a halogen, and a C₃₋₆ cycloalkyl.

35. A compound according to any of claims 1 to 34, or a pharmaceutically acceptable salt thereof, wherein the R^{a} and R^{b} of R¹ together with the N to which they are attached form a heterocyclyl, the heterocyclyl is a monocyclic or bicyclic heterocyclyl unsubstituted or substituted with 1 or 2 substituents independently selected from C₁₋₃ alkyl, halogen, and C₃₋₆ cycloalkyl.

36. A compound according to claim 35, or a pharmaceutically acceptable salt thereof, wherein the monocyclic heterocyclyl is a morpholino unsubstituted or substituted with 1-2 methyl, and the bicyclic heterocyclyl is selected from dihydropyrazolopyrimidinyl and dihydropyridooxazinyl, each are unsubstituted.

37. A compound according to any of claims 1 to 34, or a pharmaceutically acceptable salt thereof, wherein R^{a} is selected from pyridyl and pyrazolyl, each are unsubstituted or substituted with 1 or 2 substituents independently selected from C₁₋₃ alkyl, halogen, and C₃₋₆ cycloalkyl; and wherein R^{b} is hydrogen.

38. A compound according to any of claims 1 to 37, or a pharmaceutically acceptable salt thereof, wherein R^{c} is a phenyl unsubstituted or substituted with 1 or 2 substituents independently selected from C₁₋₃ alkyl and halogen.

39. A compound according to any of claims 1 to 32, or a pharmaceutically acceptable salt thereof, wherein
R¹ is selected from trifluoromethyl; morpholin-4-yl unsubstituted or substituted with 1 methyl; 6,7-dihydro-5H-pyrazolo[1,5-a]pyrimidin-4-yl; 2,3-dihydropyrido[2,3-b][1,4]oxazin-1-yl; isoxazolyl unsubstituted or substituted with 1 or 2 methyl; pyrazolyl unsubstituted or substituted with 1 or 2 substituents independently selected from methyl and cyclopropyl; pyridyl unsubstituted or substituted with 1 methyl; thiazolyl unsubstituted or substituted with 1 methyl; —CH₂-phenyl, wherein the phenyl is unsubstituted or substituted with 1 halogen; —NHR^{a}; and phenoxyl unsubstituted or substituted with 1 or 2 substituents independently selected from halogen and methyl; and
R^{a} is selected from pyrazolyl unsubstituted or substituted with 1 methyl; and pyridinyl unsubstituted or substituted with 1 halogen.

40. A compound according to any of claims 1 to 32, or a pharmaceutically acceptable salt thereof, wherein R¹ is selected from 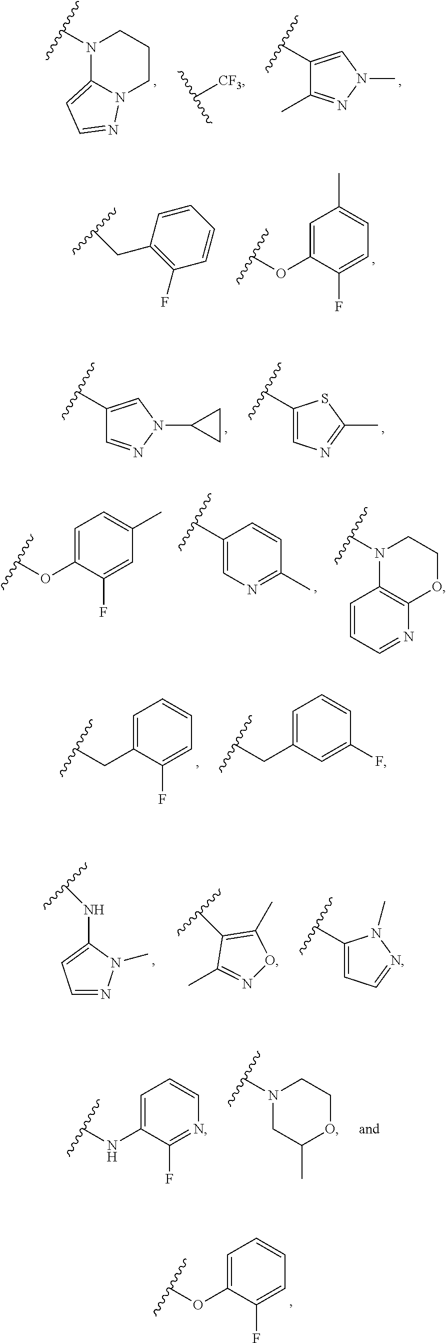
wherein 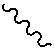
indicates the attachment point

41. A compound according to any of claims 1 to 40, or a pharmaceutically acceptable salt thereof, wherein R² is selected from hydrogen, chloro, cyclopropyl, and methyl.

42. A compound according to claim 41, or a pharmaceutically acceptable salt thereof wherein R² is hydrogen.

43. A compound according to claim 41, or a pharmaceutically acceptable salt thereof wherein R² is chloro.

44. A compound according to claim 41, or a pharmaceutically acceptable salt thereof, wherein R² is cyclopropyl.

45. A compound according to claim 41, or a pharmaceutically acceptable salt thereof, wherein R² is methyl.

46. A compound of claim 1, selected from:
5,6-dimethyl-4-[3-(trifluoromethyl)-7,8-dihydro-5H-1,6-naphthyridin-6-yl]thieno[2,3-d]pyrimidine;
2,5,6-trimethyl-4-[3-(trifluoromethyl)-7,8-dihydro-5H-1,6-naphthyridin-6-yl]thieno[2,3-d]pyrimidine;
5-methyl-4-[3-(trifluoromethyl)-7,8-dihydro-5H-1,6-naphthyridin-6-yl]furo[2,3-d]pyrimidine;
4-[3-(6,7-dihydro-5H-pyrazolo[1,5-a]pyrimidin-4-yl)-7,8-dihydro-5H-1,6-naphthyridin-6-yl]-5-methyl-furo[2,3-d]pyrimidine;
5-methyl-4-[3-(trifluoromethyl)-7,8-dihydro-5H-1,6-naphthyridin-6-yl]thieno[2,3-d]pyrimidine;
6-methyl-4-[3-(trifluoromethyl)-7,8-dihydro-5H-1,6-naphthyridin-6-yl]thieno[2,3-d]pyrimidine;
6-(2-chloro-6,7-dihydro-5H-cyclopenta[d]pyrimidin-4-yl)-3-(trifluoromethyl)-7,8-dihydro-5H-1,6-naphthyridine;
4-[3-(trifluoromethyl)-7,8-dihydro-5H-1,6-naphthyridin-6-yl]thieno[3,2-d]pyrimidine;
7-methyl-4-[3-(trifluoromethyl)-7,8-dihydro-5H-1,6-naphthyridin-6-yl]thieno[3,2-d]pyrimidine;
3-methyl-4-[3-(trifluoromethyl)-7,8-dihydro-5H-1,6-naphthyridin-6-yl]isoxazolo[5,4-d]pyrimidine;
6-(1-methylpyrazolo[4,3-d]pyrimidin-7-yl)-3-(trifluoromethyl)-7,8-dihydro-5H-1,6-naphthyridine;
4-[3-(1,3-dimethylpyrazol-4-yl)-7,8-dihydro-5H-1,6-naphthyridin-6-yl]-3-methyl-isoxazolo[5,4-d]pyrimidine;
4-[3-(1,3-dimethylpyrazol-4-yl)-7,8-dihydro-5H-1,6-naphthyridin-6-yl]-5-methyl-furo[2,3-d]pyrimidine;
4-[3-(6,7-dihydro-5H-pyrazolo[1,5-a]pyrimidin-4-yl)-7,8-dihydro-5H-1,6-naphthyridin-6-yl]-3-methyl-isoxazolo[5,4-d]pyrimidine;
4-[3-(1-cyclopropylpyrazol-4-yl)-7,8-dihydro-5H-1,6-naphthyridin-6-yl]-5-methyl-furo[2,3-d]pyrimidine;
4-[3-(1-cyclopropylpyrazol-4-yl)-7,8-dihydro-5H-1,6-naphthyridin-6-yl]-3-methyl-isoxazolo[5,4-d]pyrimidine;
6-(7-methylpyrrolo[2,3-d]pyrimidin-4-yl)-3-(trifluoromethyl)-7,8-dihydro-5H-1,6-naphthyridine;
4-[3-(1,3-dimethylpyrazol-4-yl)-7,8-dihydro-5H-1,6-naphthyridin-6-yl]-6-methyl-thieno[2,3-d]pyrimidine;
1-[6-(6-methylthieno[2,3-d]pyrimidin-4-yl)-7,8-dihydro-5H-1,6-naphthyridin-3-yl]-2,3-dihydropyrido[2,3-b][1,4]oxazine;
6-methyl-4-[3-(6-methyl-3-pyridyl)-7,8-dihydro-5H-1,6-naphthyridin-6-yl]thieno[2,3-d]pyrimidine;
7-methyl-4-[3-(6-methyl-3-pyridyl)-7,8-dihydro-5H-1,6-naphthyridin-6-yl]thieno[3,2-d]pyrimidine;
6-(2-cyclopropyl-6,7-dihydro-5H-cyclopenta[d]pyrimidin-4-yl)-3-(trifluoromethyl)-7,8-dihydro-5H-1,6-naphthyridine;
6-(2-methyl-6,7-dihydro-5H-cyclopenta[d]pyrimidin-4-yl)-3-(trifluoromethyl)-7,8-dihydro-5H-1,6-naphthyridine;
1-[6-(5-methylfuro[2,3-d]pyrimidin-4-yl)-7,8-dihydro-5H-1,6-naphthyridin-3-yl]-2,3-dihydropyrido[2,3-b][1,4]oxazine;
5-methyl-4-[3-(6-methyl-3-pyridyl)-7,8-dihydro-5H-1,6-naphthyridin-6-yl]furo[2,3-d]pyrimidine;
5-methyl-4-[3-(2-methylthiazol-5-yl)-7,8-dihydro-5H-1,6-naphthyridin-6-yl]furo[2,3-d]pyrimidine;
1-[6-(5-methylthieno[2,3-d]pyrimidin-4-yl)-7,8-dihydro-5H-1,6-naphthyridin-3-yl]-2,3-dihydropyrido[2,3-b][1,4]oxazine;
5-methyl-4-[3-(6-methyl-3-pyridyl)-7,8-dihydro-5H-1,6-naphthyridin-6-yl]thieno[2,3-d]pyrimidine;
5-methyl-4-[3-(2-methylthiazol-5-yl)-7,8-dihydro-5H-1,6-naphthyridin-6-yl]thieno[2,3-d]pyrimidine;
1-[6-(3-methylisoxazolo[5,4-d]pyrimidin-4-yl)-7,8-dihydro-5H-1,6-naphthyridin-3-yl]-2,3-dihydropyrido[2,3-b][1,4]oxazine;
3-methyl-4-[3-(6-methyl-3-pyridyl)-7,8-dihydro-5H-1,6-naphthyridin-6-yl]isoxazolo[5,4-d]pyrimidine;
3-methyl-4-[3-(2-methylthiazol-5-yl)-7,8-dihydro-5H-1,6-naphthyridin-6-yl]isoxazolo[5,4-d]pyrimidine;
4-[3-(2-fluoro-4-methyl-phenoxy)-7,8-dihydro-5H-1,6-naphthyridin-6-yl]-5-methyl-thieno[2,3-d]pyrimidine;
4-[3-(6,7-dihydro-5H-pyrazolo[1,5-a]pyrimidin-4-yl)-7,8-dihydro-5H-1,6-naphthyridin-6-yl]-6-methyl-thieno[2,3-d]pyrimidine;
4-[3-(1-cyclopropylpyrazol-4-yl)-7,8-dihydro-5H-1,6-naphthyridin-6-yl]-6-methyl-thieno[2,3-d]pyrimidine;
6-methyl-4-[3-(2-methylthiazol-5-yl)-7,8-dihydro-5H-1,6-naphthyridin-6-yl]thieno[2,3-d]pyrimidine;
4-[3-(6,7-dihydro-5H-pyrazolo[1,5-a]pyrimidin-4-yl)-7,8-dihydro-5H-1,6-naphthyridin-6-yl]-7-methyl-thieno[3,2-d]pyrimidine;
4-[3-(1-cyclopropylpyrazol-4-yl)-7,8-dihydro-5H-1,6-naphthyridin-6-yl]-7-methyl-thieno[3,2-d]pyrimidine;
4-[3-(1,3-dimethylpyrazol-4-yl)-7,8-dihydro-5H-1,6-naphthyridin-6-yl]-7-methyl-thieno[3,2-d]pyrimidine;
1-[6-(7-methylthieno[3,2-d]pyrimidin-4-yl)-7,8-dihydro-5H-1,6-naphthyridin-3-yl]-2,3-dihydropyrido[2,3-b][1,4]oxazine;
7-methyl-4-[3-(2-methylthiazol-5-yl)-7,8-dihydro-5H-1,6-naphthyridin-6-yl]thieno[3,2-d]pyrimidine;
4-[3-(6,7-dihydro-5H-pyrazolo[1,5-a]pyrimidin-4-yl)-7,8-dihydro-5H-1,6-naphthyridin-6-yl]-5-methyl-thieno[2,3-d]pyrimidine;
4-[3-(1-cyclopropylpyrazol-4-yl)-7,8-dihydro-5H-1,6-naphthyridin-6-yl]-5-methyl-thieno[2,3-d]pyrimidine;
4-[3-(2-fluoro-5-methyl-phenoxy)-7,8-dihydro-5H-1,6-naphthyridin-6-yl]thieno[3,2-d]pyrimidine;
4-[3-[(2-fluorophenyl)methyl]-7,8-dihydro-5H-1,6-naphthyridin-6-yl]-5-methyl-furo[2,3-d]pyrimidine;
4-[3-(1,3-dimethylpyrazol-4-yl)-7,8-dihydro-5H-1,6-naphthyridin-6-yl]-5-methyl-thieno[2,3-d]pyrimidine;
5-bromo-4-[3-(trifluoromethyl)-7,8-dihydro-5H-1,6-naphthyridin-6-yl]thieno[2,3-d]pyrimidine;
6-bromo-4-[3-(trifluoromethyl)-7,8-dihydro-5H-1,6-naphthyridin-6-yl]thieno[2,3-d]pyrimidine;
4-[3-(6,7-dihydro-5H-pyrazolo[1,5-a]pyrimidin-4-yl)-7,8-dihydro-5H-1,6-naphthyridin-6-yl]-6-isopropyl-thieno[2,3-d]pyrimidine;
4-[3-[(3-fluorophenyl)methyl]-7,8-dihydro-5H-1,6-naphthyridin-6-yl]-5-methyl-furo[2,3-d]pyrimidine;
4-[3-(2-fluorophenoxy)-7,8-dihydro-5H-1,6-naphthyridin-6-yl]-5-methyl-thieno[2,3-d]pyrimidine;
4-[3-(2-fluorophenoxy)-7,8-dihydro-5H-1,6-naphthyridin-6-yl]thieno[2,3-d]pyrimidine;
4-[3-(2-methylthiazol-5-yl)-7,8-dihydro-5H-1,6-naphthyridin-6-yl]thieno[2,3-d]pyrimidine;
N-(2-methylpyrazol-3-yl)-6-thieno[2,3-d]pyrimidin-4-yl-7,8-dihydro-5H-1,6-naphthyridin-3-amine;
4-[6-(6-isopropylthieno[2,3-d]pyrimidin-4-yl)-7,8-dihydro-5H-1,6-naphthyridin-3-yl]-3,5-dimethyl-isoxazole;
5-deuterio-4-[3-(trifluoromethyl)-7,8-dihydro-5H-1,6-naphthyridin-6-yl]thieno[2,3-d]pyrimidine;
4-[3-(2-fluorophenoxy)-7,8-dihydro-5H-1,6-naphthyridin-6-yl]-6-methyl-thieno[2,3-d]pyrimidine;
5-cyclopropyl-4-[3-(trifluoromethyl)-7,8-dihydro-5H-1,6-naphthyridin-6-yl]thieno[2,3-d]pyrimidine;
6-(5-methylfuro[2,3-d]pyrimidin-4-yl)-N-(2-methylpyrazol-3-yl)-7,8-dihydro-5H-1,6-naphthyridin-3-amine;
N-(2-fluoro-3-pyridyl)-6-(5-methylfuro[2,3-d]pyrimidin-4-yl)-7,8-dihydro-5H-1,6-naphthyridin-3-amine;
5-methyl-4-[3-(2-methylpyrazol-3-yl)-7,8-dihydro-5H-1,6-naphthyridin-6-yl]furo[2,3-d]pyrimidine;
2-chloro-4-[3-(trifluoromethyl)-7,8-dihydro-5H-1,6-naphthyridin-6-yl]-5,7-dihydrofuro[3,4-d]pyrimidine;
1-[6-(2-chloro-5,7-dihydrofuro[3,4-d]pyrimidin-4-yl)-7,8-dihydro-5H-1,6-naphthyridin-3-yl]-2,3-dihydropyrido[2,3-b][1,4]oxazine;
2-chloro-4-(3-(6,7-dihydropyrazolo[1,5-a]pyrimidin-4(5H)-yl)-7,8-dihydro-1,6-naphthyridin-6(5H)-yl)-5,7-dihydrofuro[3,4-d]pyrimidine;
(R)-2-chloro-4-(3-(2-methylmorpholino)-7,8-dihydro-1,6-naphthyridin-6(5H)-yl)-5,7-dihydrofuro[3,4-d]pyrimidine;
2-chloro-4-(3-cyclopropyl-7,8-dihydro-1,6-naphthyridin-6(5H)-yl)-5,7-dihydrofuro[3,4-d]pyrimidine;
6-(2-chloro-5,7-dihydrofuro[3,4-d]pyrimidin-4-yl)-N-(1-methyl-1H-pyrazol-5-yl)-5,6,7,8-tetrahydro-1,6-naphthyridin-3-amine;
6-(2-chloro-5,7-dihydrofuro[3,4-d]pyrimidin-4-yl)-N-(2-fluoropyridin-3-yl)-5,6,7,8-tetrahydro-1,6-naphthyridin-3-amine; and
2-chloro-4-(3-(2-fluorophenoxy)-7,8-dihydro-1,6-naphthyridin-6(5H)-yl)-5,7-dihydrofuro[3,4-d]pyrimidine, or a pharmaceutically acceptable salt thereof.

47. The compound of any one of claims 1 to 46, or a pharmaceutically acceptable salt thereof, wherein the compound is isotopically labeled.

48. A pharmaceutical composition comprising a compound of any one of claims 1 to 47, or a pharmaceutically acceptable salt thereof, and a pharmaceutically acceptable carrier.

49. A method for treating a neurological and/or psychiatric disorder associated with muscarinic acetylcholine receptor dysfunction in a patient in need thereof, comprising a step of administering to the patient a therapeutically effective amount of a compound, or pharmaceutically acceptable salt thereof, or pharmaceutical composition according to any one of claims 1 to 48.

50. The method of claim 49, wherein the disorder is selected from Alzheimer's disease, schizophrenia, a sleep disorder, borderline personality disorder, Tourette's syndrome, bipolar disorder, tardive dyskinesia, Huntington's disease, a pain disorder, and a cognitive disorder.

51. The method of claim 50, wherein the disorder is Alzheimer's disease.

52. The method of claim 50, wherein the disorder is schizophrenia.

53. The method of claim 49, wherein the disorder is selected from psychosis, schizophrenia, conduct disorder, disruptive behavior disorder, bipolar disorder, psychotic episodes of anxiety, anxiety associated with psychosis, psychotic mood disorders such as severe major depressive disorder; mood disorders associated with psychotic disorders, acute mania, depression associated with bipolar disorder, mood disorders associated with schizophrenia, behavioral manifestations of mental retardation, autistic disorder, movement disorders, Tourette's syndrome, akinetic-rigid syndrome, movement disorders associated with Parkinson's disease, tardive dyskinesia, drug induced and neurodegeneration based dyskinesias, attention deficit hyperactivity disorder, cognitive disorders, dementias, and memory disorders.

54. A compound, or a pharmaceutically acceptable salt thereof according to claims 1 to 47 for use in therapy.

55. A compound, or a pharmaceutically acceptable salt thereof, or a pharmaceutical composition according to any one of claims 1 to 48 for use in the treatment of a disorder selected from Alzheimer's disease, schizophrenia, borderline personality disorder, Tourette's syndrome, bipolar disorder, tardive dyskinesia, Huntington's disease, a sleep disorder, a pain disorder, and a cognitive disorder.

56. A compound, or a pharmaceutically acceptable salt thereof, or a pharmaceutical composition according to any one of claims 1 to 48 for use in the treatment of a disorder selected from psychosis, schizophrenia, conduct disorder, disruptive behavior disorder, bipolar disorder, psychotic episodes of anxiety, anxiety associated with psychosis, psychotic mood disorders such as severe major depressive disorder; mood disorders associated with psychotic disorders, acute mania, depression associated with bipolar disorder, mood disorders associated with schizophrenia, behavioral manifestations of mental retardation, autistic disorder, movement disorders, Tourette's syndrome, akinetic-rigid syndrome, movement disorders associated with Parkinson's disease, tardive dyskinesia, drug induced and neurodegeneration based dyskinesias, attention deficit hyperactivity disorder, cognitive disorders, dementias, and memory disorders.

57. Use of a compound, or a pharmaceutically acceptable salt thereof, or a pharmaceutical composition according to any one of claims 1 to 48 in the manufacture of a medicament for the treatment of a disorder selected from Alzheimer's disease, schizophrenia, borderline personality disorder, Tourette's syndrome, bipolar disorder, tardive dyskinesia, Huntington's disease a sleep disorder, a pain disorder, and a cognitive disorder.

58. Use of a compound, or a pharmaceutically acceptable salt thereof, or a pharmaceutical composition according to any one of claims 1 to 48 in the manufacture of a medicament for the treatment of a disorder selected from psychosis, schizophrenia, conduct disorder, disruptive behavior disorder, bipolar disorder, psychotic episodes of anxiety, anxiety associated with psychosis, psychotic mood disorders such as severe major depressive disorder; mood disorders associated with psychotic disorders, acute mania, depression associated with bipolar disorder, mood disorders associated with schizophrenia, behavioral manifestations of mental retardation, autistic disorder, movement disorders, Tourette's syndrome, akinetic-rigid syndrome, movement disorders associated with Parkinson's disease, tardive dyskinesia, drug induced and neurodegeneration based dyskinesias, attention deficit hyperactivity disorder, cognitive disorders, dementias, and memory disorders.

59. A kit comprising a compound, or a pharmaceutically acceptable salt thereof, or a pharmaceutical composition of any one of claims 1 to 48, and one or more of: (a) at least one agent known to increase mAChR M₄ activity; (b) at least one agent known to decrease mAChR M₄ activity; (c) at least one agent known to treat a disorder associated with cholinergic activity; (d) instructions for treating a disorder associated with cholinergic activity; (e) instructions for treating a disorder associated with mAChR M₄ receptor activity; and (f) instructions for administering the compound in connection with cognitive or behavioral therapy.
